(19) United States
(12) Patent Application Publication
(10) Pub. No.: US 2023/0248368 A1
(43) Pub. Date: Aug. 10, 2023

(54) Intrasacular Aneurysm Occlusion Device with a Proximal Bowl-Shaped Mesh and a Distal Globular Mesh

(71) Applicant: Robert A. Connor, St. Paul, MN (US)

(72) Inventor: Robert A. Connor, St. Paul, MN (US)

(73) Assignee: Aneuclose LLC, St. Paul, MN (US)

(21) Appl. No.: 18/135,153

(22) Filed: Apr. 15, 2023

Related U.S. Application Data
(63) Continuation-in-part of application No. 17/970,510, filed on Oct. 20, 2022.
Application No. 17/970,510 is a continuation-in-part of application No. 17/965,502, filed on Oct. 13, 2022.
Application No. 17/965,502 is a continuation-in-part of application No. 17/829,313, filed on May 31, 2022.
Application No. 17/970,510 is a continuation-in-part of application No. 17/965,502, filed on Oct. 13, 2022.
Application No. 17/965,502 is a continuation-in-part of application No. 17/829,313, filed on May 31, 2022.
Application No. 17/829,313 is a continuation-in-part of application No. 17/476,845, filed on Sep. 16, 2021, now Pat. No. 11,583,289.
Application No. 17/965,502 is a continuation-in-part of application No. 17/829,313, filed on May 31, 2022.
Application No. 17/829,313 is a continuation of application No. 17/476,845, filed on Sep. 16, 2021, now Pat. No. 11,583,289.
Application No. 17/829,313 is a continuation-in-part of application No. 17/485,390, filed on Sep. 25, 2021, now Pat. No. 11,471,164.
Application No. 17/485,390 is a continuation-in-part of application No. 17/476,845, filed on Sep. 16, 2021, now Pat. No. 11,583,289.
Application No. 17/476,845 is a continuation-in-part of application No. 17/472,674, filed on Sep. 12, 2021, now abandoned.
Application No. 17/472,674 is a continuation-in-part of application No. 17/467,680, filed on Sep. 7, 2021, now abandoned.
Application No. 17/467,680 is a continuation-in-part of application No. 17/466,497, filed on Sep. 3, 2021, now Pat. No. 11,357,511.
Application No. 17/466,497 is a continuation-in-part of application No. 17/353,652, filed on Jun. 21, 2021, now abandoned.
Application No. 17/353,652 is a continuation-in-part of application No. 17/220,002, filed on Apr. 1, 2021, now Pat. No. 11,464,518.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/211,446 is a continuation-in-part of application No. 16/693,267, filed on Nov. 23, 2019, now abandoned.
Application No. 16/693,267 is a continuation-in-part of application No. 16/660,929, filed on Oct. 23, 2019, now Pat. No. 11,471,163.
Application No. 16/693,267 is a continuation-in-part of application No. 16/660,929, filed on Oct. 23, 2019, now Pat. No. 11,471,163.
Application No. 16/660,929 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 15/865,822, filed on Jan. 9, 2018, now Pat. No. 10,716,573.
Application No. 15/865,822 is a continuation-in-part of application No. 15/081,909, filed on Mar. 27, 2016, now abandoned.
Application No. 15/081,909 is a continuation-in-part of application No. 14/526,600, filed on Oct. 29, 2014, now abandoned.
Application No. 14/526,600 is a continuation-in-part of application No. 15/080,915, filed on Mar. 25, 2016, now Pat. No. 10,028,747.
Application No. 15/080,915 is a continuation-in-part of application No. 14/526,600, filed on Oct. 29, 2014, now abandoned.
Application No. 15/081,909 is a continuation-in-part of application No. 14/526,600, filed on Oct. 29, 2014, now abandoned.
Application No. 15/080,915 is a continuation-in-part of application No. 14/526,600, filed on Oct. 29, 2014, now abandoned.
Application No. 14/526,600 is a continuation-in-part of application No. 12/989,048, filed on Oct. 21, 2010, now Pat. No. 8,974,487.
(60) Provisional application No. 63/119,774, filed on Dec. 1, 2020.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/720,173, filed on Aug. 21, 2018.
(60) Provisional application No. 62/589,754, filed on Nov. 22, 2017.
(60) Provisional application No. 62/472,519, filed on Mar. 16, 2017.
(60) Provisional application No. 62/589,754, filed on Nov. 22, 2017.
(60) Provisional application No. 62/472,519, filed on Mar. 16, 2017.
(60) Provisional application No. 62/444,860, filed on Jan. 11, 2017.
(60) Provisional application No. 61/897,245, filed on Oct. 30, 2013.
(60) Provisional application No. 61/126,047, filed on May 1, 2008.
(60) Provisional application No. 61/126,027, filed on May 1, 2008.

(51) Int. Cl. A61B 17/12; A61B 34/00

(52) U.S. Cl. CPC A61B 17/12172; A61B 34/73; A61B 17/12113; A61B 2017/00323

(57) ABSTRACT

Disclosed herein is an intrasacular aneurysm occlusion device with a proximal bowl-shaped mesh which covers the neck of an aneurysm sac and a distal globular mesh between the proximal bowl-shaped mesh and the dome of the aneurysm sac. The proximal bowl-shaped mesh blocks blood flow into the aneurysm sac and the distal globular mesh holds the bowl-shaped mesh in place against the aneurysm neck.

### CROSS-REFERENCE TO RELATED APPLICATIONS

This application is a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31. U.S. patent application Ser. No. 17/970,510 was a continuation-in-part of U.S. patent application Ser. No. 17/965,502 filed on 2022 Oct. 13, a continuation-in-part of U.S. patent application Ser. No. 17/829,313 filed on 2022 May 31, and a continuation-in-part of U.S. patent application Ser. No. 17/476,845 filed on 2021 Sep. 16.

U.S. patent application Ser. No. 17/829,313 was a continuation-in-part of U.S. patent application Ser. No. 17/485,390 filed on 2021 Sep. 25, was a continuation-in-part of U.S. patent application Ser. No. 17/476,845 filed on 2021 Sep. 16, was a continuation-in-part of U.S. patent application Ser. No. 17/472,674 filed on 2021 Sep. 12, was a continuation-in-part of U.S. patent application Ser. No. 17/467,680 filed on 2021 Sep. 7, was a continuation-in-part of U.S. patent application Ser. No. 17/466,497 filed on 2021 Sep. 3, was a continuation-in-part of U.S. patent application Ser. No. 17/353,652 filed on 2021 Jun. 21, was a continuation-in-part of U.S. patent application Ser. No. 17/220,002 filed on 2021 Apr. 1, was a continuation-in-part of U.S. patent application Ser. No. 17/214,827 filed on 2021 Mar. 27, was a continuation-in-part of U.S. patent application Ser. No. 17/211,446 filed on 2021 Mar. 24, was a continuation-in-part of U.S. patent application Ser. No. 16/693,267 filed on 2019 Nov. 23, and was a continuation-in-part of U.S. patent application Ser. No. 16/660,929 filed on 2019 Oct. 23.

U.S. patent application Ser. No. 17/220,002 was a continuation-in-part of U.S. patent application Ser. No. 17/214,827 filed on 2021 Mar. 27. U.S. patent application Ser. No. 17/220,002 was a continuation-in-part of U.S. patent application Ser. No. 17/211,446 filed on 2021 Mar. 24. U.S. patent application Ser. No. 17/220,002 claimed the priority benefit of U.S. provisional patent application 63/119,774 filed on 2020 Dec. 1. U.S. patent application Ser. No. 17/220,002 was a continuation-in-part of U.S. patent application Ser. No. 16/693,267 filed on 2019 Nov. 23. U.S. patent application Ser. No. 17/220,002 was a continuation-in-part of U.S. patent application Ser. No. 16/660,929 filed on 2019 Oct. 23.

U.S. patent application Ser. No. 16/693,267 was a continuation-in-part of U.S. patent application Ser. No. 16/660,929 filed on 2019 Oct. 23. U.S. patent application Ser. No. 16/693,267 claimed the priority benefit of U.S. provisional patent application 62/794,609 filed on 2019 Jan. 19. U.S. patent application Ser. No. 16/693,267 claimed the priority benefit of U.S. provisional patent application 62/794,607 filed on 2019 Jan. 19. U.S. patent application Ser. No. 16/693,267 was a continuation-in-part of U.S. patent application Ser. No. 16/541,241 filed on 2019 Aug. 15. U.S. patent application Ser. No. 16/693,267 was a continuation-in-part of U.S. patent application Ser. No. 15/865,822 filed on 2018 Jan. 9 which issued as U.S. patent Ser. No. 10/716,573 on 2020 Jul. 21. U.S. patent application Ser. No. 16/693,267 was a continuation-in-part of U.S. patent application Ser. No. 15/861,482 filed on 2018 Jan. 3.

U.S. patent application Ser. No. 16/660,929 claimed the priority benefit of U.S. provisional patent application 62/794,609 filed on 2019 Jan. 19. U.S. patent application Ser. No. 16/660,929 claimed the priority benefit of U.S. provisional patent application 62/794,607 filed on 2019 Jan. 19. U.S. patent application Ser. No. 16/660,929 was a continuation-in-part of U.S. patent application Ser. No. 16/541,241 filed on 2019 Aug. 15. U.S. patent application Ser. No. 16/660,929 was a continuation-in-part of U.S. patent application Ser. No. 15/865,822 filed on 2018 Jan. 9 which issued as U.S. patent Ser. No. 10/716,573 on 2020 Jul. 21. U.S. patent application Ser. No. 16/660,929 was a continuation-in-part of U.S. patent application Ser. No. 15/861,482 filed on 2018 Jan. 3.

U.S. patent application Ser. No. 16/541,241 claimed the priority benefit of U.S. provisional patent application 62/794,609 filed on 2019 Jan. 19. U.S. patent application Ser. No. 16/541,241 claimed the priority benefit of U.S. provisional patent application 62/794,607 filed on 2019 Jan. 19. U.S. patent application Ser. No. 16/541,241 claimed the priority benefit of U.S. provisional patent application 62/720,173 filed on 2018 Aug. 21. U.S. patent application Ser. No. 16/541,241 was a continuation-in-part of U.S. patent application Ser. No. 15/865,822 filed on 2018 Jan. 9 which issued as U.S. patent Ser. No. 10/716,573 on 2020 Jul. 21

U.S. patent application Ser. No. 15/865,822 claimed the priority benefit of U.S. provisional patent application 62/589,754 filed on 2017 Nov. 22. U.S. patent application Ser. No. 15/865,822 claimed the priority benefit of U.S. provisional patent application 62/472,519 filed on 2017 Mar. 16. U.S. patent application Ser. No. 15/865,822 was a continuation-in-part of U.S. patent application Ser. No. 15/081,909 filed on 2016 Mar. 27. U.S. patent application Ser. No. 15/865,822 was a continuation-in-part of U.S. patent application Ser. No. 14/526,600 filed on 2014 Oct. 29.

U.S. patent application Ser. No. 15/861,482 claimed the priority benefit of U.S. provisional patent application 62/589,754 filed on 2017 Nov. 22. U.S. patent application Ser. No. 15/861,482 claimed the priority benefit of U.S. provisional patent application 62/472,519 filed on 2017 Mar. 16. U.S. patent application Ser. No. 15/861,482 claimed the priority benefit of U.S. provisional patent application 62/444,860 filed on 2017 Jan. 11. U.S. patent application Ser. No. 15/861,482 was a continuation-in-part of U.S. patent application Ser. No. 15/080,915 filed on 2016 Mar. 25 which issued as U.S. patent Ser. No. 10/028,747 on 2018 Jul. 24. U.S. patent application Ser. No. 15/861,482 was a continuation-in-part of U.S. patent application Ser. No. 14/526,600 filed on 2014 Oct. 29.

U.S. patent application Ser. No. 15/081,909 was a continuation-in-part of U.S. patent application Ser. No. 14/526,600 filed on 2014 Oct. 29. U.S. patent application Ser. No. 15/080,915 was a continuation-in-part of U.S. patent application Ser. No. 14/526,600 filed on 2014 Oct. 29. U.S. patent application Ser. No. 14/526,600 claimed the priority benefit of U.S. provisional patent application 61/897,245 filed on 2013 Oct. 30. U.S. patent application Ser. No. 14/526,600 was a continuation-in-part of U.S. patent application Ser. No. 12/989,048 filed on 2010 Oct. 21 which issued as U.S. Pat. No. 8,974,487 on 2015 Mar. 10. U.S. patent application Ser. No. 12/989,048 claimed the priority benefit of U.S. provisional patent application 61/126,047 filed on 2008 May 1. U.S. patent application Ser. No. 12/989,048 claimed the priority benefit of U.S. provisional patent application 61/126,027 filed on 2008 May 1.

The entire contents of these related applications are incorporated herein by reference.

### FEDERALLY SPONSORED RESEARCH

Not Applicable

### SEQUENCE LISTING OR PROGRAM

Not Applicable

### BACKGROUND—FIELD OF INVENTION

This invention relates to devices and methods for occluding a cerebral aneurysm.

### INTRODUCTION

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Sadly, even among those who survive, approximately one-half suffer significant and permanent deterioration of brain function. Better alternatives for cerebral aneurysm treatment are needed.

### REVIEW OF THE RELEVANT ART

U.S. patent application 20190192168 (Lorenzo et al., Jun. 27, 2019, “Aneurysm Device and Delivery Method”) and U.S. patent Ser. No. 10/716,574 (Lorenzo et al., Jul. 21, 2020, “Aneurysm Device and Delivery Method”) discloses a self-expanding braid for treating an aneurysm, including a method for inverting and buckling a proximal segment. U.S. patent application 20190223878 (Lorenzo et al., Jul. 25, 2019, “Aneurysm Device and Delivery System”) and U.S. patent application 20200397447 (Lorenzo et al., Dec. 24, 2020, “Aneurysm Device and Delivery System”) discloses an expandable segment which radially expands inside an outer occlusive sack. U.S. patent application 20190365385 (Gorochow et al., Dec. 5, 2019, “Aneurysm Device and Delivery System”) and U.S. patent Ser. No. 10/939,915 (Gorochow et al., Mar. 9, 2021, “Aneurysm Device and Delivery System”) discloses a braid, wherein translating the braid causes a delivery portion to expand and form a distal sack as well as invert into itself.

U.S. patent application 20200375607 (Soto Del Valle et al., Dec. 3, 2020, “Aneurysm Device and Delivery System”) discloses a method of expanding mesh segments to form an outer occlusive sack and an inner occlusive sack. U.S. patent Ser. No. 10/905,430 (Lorenzo et al., Feb. 2, 2021, “Aneurysm Device and Delivery System”) discloses an expandable segment which radially expands inside an outer occlusive sack. U.S. patent Ser. No. 11/058,430 (Gorochow et al., Jul. 13, 2021, “Aneurysm Device and Delivery System”) discloses a braid with a proximal expandable portion for positioning inside an aneurysm and sealing across the neck of the aneurysm.

U.S. patent application 20200375606 (Lorenzo, Dec. 3, 2020, “Aneurysm Method and System”) discloses a braided implant which is invertible about the distal implant end. U.S. patent application 20210177429 (Lorenzo, Jun. 17, 2021, “Aneurysm Method and System”) discloses a vaso-occlusive device with at least two nested sacks. U.S. patent application 20210330331 (Lorenzo, Oct. 28, 2021, “Aneurysm Occlusion Device”) and U.S. patent Ser. No. 11/154,302 (Lorenzo et al., Oct. 26, 2021, “Aneurysm Occlusion Device”) disclose an occlusion device with a substantially annular body disposed on the proximal end region of the device. U.S. patent application 20200305886 (Soto Del Valle et al, Oct. 1, 2020, “Aneurysm Treatment Device”) and U.S. patent application 20220225997 (Soto Del Valle et al., Jul. 21, 2022, “Aneurysm Treatment Device”) disclose a device with an expandable sack with a free open end and an elongated looping portion.

U.S. patent application 20200367906 (Xu et al., Nov. 26, 2020, “Aneurysm Treatment With Pushable Ball Segment”) and U.S. patent application 20230016312 (Xu et al., Jan. 19, 2023, “Aneurysm Treatment with Pushable Implanted Braid”) disclose a braided implant with a retractable dual proximal layer. U.S. patent application 20210244420 (Aboytes et al., Aug. 12, 2021, “Devices and Methods for the Treatment of Vascular Defects”) discloses aneurysm occlusion devices with a first configuration in which a first portion and a second portion are substantially linearly aligned and a second configuration in which the second portion at least partially overlaps the first portion.

U.S. patent application 20160249935˜Hewitt et al., Sep. 1, 2016, “Devices for Therapeutic Vascular Procedures”), U.S. patent application 20160367260 (Hewitt et al., Dec. 22, 2016, “Devices for Therapeutic Vascular Procedures”), U.S. Pat. No. 9,629,635 (Hewitt et al., Apr. 25, 2017, “Devices for Therapeutic Vascular Procedures”), and U.S. patent application 20170128077 (Hewitt et al., May 11, 2017, “Devices for Therapeutic Vascular Procedures”) disclose a self-expanding resilient permeable shell and a metallic coil secured to the distal end of the permeable shell. U.S. patent application 20190223881 (Hewitt et al., Jul. 25, 2019, “Devices for Therapeutic Vascular Procedures”) discloses a self-expanding resilient permeable shell made from elongate resilient filaments with a distal region that extends beyond the distal end of the permeable shell.

U.S. patent application 20210346032 (Patterson et al., Nov. 11, 2021, “Devices for Treatment of Vascular Defects”) discloses an expandable stent for placement in a parent vessel proximal, near, or adjacent an aneurysm. U.S. patent application 20170224350 (Shimizu et al., Aug. 10, 2017, “Devices for Vascular Occlusion”), U.S. patent Ser. No. 10/729,447 (Shimizu et al., Aug. 4, 2020, “Devices for Vascular Occlusion”), U.S. patent application 20200323534 (Shimizu et al., Oct. 15, 2020, “Devices for Vascular Occlusion”), U.S. patent Ser. No. 10/980,545 (Bowman et al., Apr. 20, 2021, “Devices for Vascular Occlusion”), U.S. patent application 20210228214 (Bowman et al., Jul. 29, 2021, “Devices for Vascular Occlusion”), and U.S. patent application 20210228214 (Bowman et al., Jul. 29, 2021, “Devices for Vascular Occlusion”) disclose an occlusive device, an occlusive device delivery system, method of using, method of delivering an occlusive device, and method of making an occlusive device to treat various intravascular conditions.

U.S. patent application 20210282785 (Dholakia et al., Sep. 16, 2021, “Devices Having Multiple Permeable Shells for Treatment of Vascular Defects”) a device with a plurality of permeable shells connected by a plurality of coils. U.S. patent application 20200187953 (Hamel et al., Jun. 18, 2020, “Devices, Systems, and Methods for the Treatment of Vascular Defects”) discloses a mesh comprising a first end portion, a second end portion, and a length extending between the first and second end portions, and a first lateral edge, a second lateral edge, and a width extending between the first and second lateral edges. U.S. patent application 20200205841 (Aboytes et al., Jul. 2, 2020, “Devices, Systems, and Methods for the Treatment of Vascular Defects”) and U.S. patent application 20210378681 (Aboytes et al., Dec. 9, 2021, “Devices, Systems, and Methods for the Treatment of Vascular Defects”) disclose aneurysm occlusion devices with a first configuration in which a first portion and a second portion are substantially linearly aligned and a second configuration in which the second portion at least partially overlaps the first portion.

U.S. patent application 20210129275 (Nguyen et al., May 6, 2021, “Devices, Systems, and Methods for Treating Aneurysms”) discloses methods of manufacturing an occlusive device including conforming a mesh to a forming assembly and setting a shape of the mesh based on the forming assembly. U.S. patent application 20210128162 (Rhee et al., May 6, 2021, “Devices, Systems, and Methods for Treatment of Intracranial Aneurysms”) discloses introduction of an embolic element to a space between an occlusive member and an inner surface of the aneurysm wall. U.S. patent application 20210128169 (Li et al., May 6, 2021, “Devices, Systems, and Methods for Treatment of Intracranial Aneurysms”) and U.S. patent application 20210153872 (Nguyen et al., May 27, 2021, “Devices, Systems, and Methods for Treatment of Intracranial Aneurysms”) disclose delivering an occlusive member to an aneurysm cavity and deforming a shape of the occlusive member via introduction of an embolic element to a space between the occlusive member and an inner surface of the aneurysm wall.

U.S. patent application 20210338247 (Gorochow, Nov. 4, 2021, “Double Layer Braid”) discloses a double layered braid for treating an aneurysm. U.S. patent application 20170281194 (Divino et al., Oct. 5, 2017, “Embolic Medical Devices”) discloses an occlusive device with an elongate member having opposing first and second side edges which extend longitudinally along the member and a member width, wherein this member has a collapsed configuration in which the first and second side edges are curled toward each other about a longitudinal axis of the member.

U.S. patent application 20140330299 (Rosenbluth et al., Nov. 6, 2014, “Embolic Occlusion Device and Method”), U.S. patent application 20180303486 (Rosenbluth et al., Oct. 25, 2018, “Embolic Occlusion Device and Method”), and U.S. patent application 20210259699 (Rosenbluth et al., Aug. 26, 2021, “Embolic Occlusion Device and Method”) disclose an occlusion device with a tubular braided member having a first end and a second end and extending along a longitudinal axis, the tubular braided member having a repeating pattern of larger diameter portions and smaller diameter portions arrayed along the longitudinal axis.

U.S. patent application 20210275187 (Franano et al., Sep. 9, 2021, “Expandable Body Device and Method of Use”) discloses medical devices comprising a single-lobed, thin-walled, expandable body. U.S. patent application 20220031334 (Aguilar, Feb. 3, 2022, “Expandable Devices for Treating Body Lumens”) discloses an occlusive device comprising an expandable mesh including an outer mesh and an inner mesh disposed within the outer mesh. U.S. patent Ser. No. 11/426,175 (Morita et al., Aug. 30, 2022, “Expansile Member”) discloses an occlusive system comprising: a catheter; a shell deliverable through the catheter, a delivery pusher detachably connected to the shell and configured to navigate the shell through the catheter, wherein the shell has a globular shaped portion.

U.S. patent application 20140358178 (Hewitt et al., Dec. 4, 2014, “Filamentary Devices for Treatment of Vascular Defects”), U.S. Pat. No. 9,078,658 (Hewitt et al., Jul. 14, 2015, “Filamentary Devices for Treatment of Vascular Defects”), U.S. patent application 20160249934 (Hewitt et al., Sep. 1, 2016, “Filamentary Devices for Treatment of Vascular Defects”), U.S. Pat. No. 9,955,976 (Hewitt et al., May 1, 2018, “Filamentary Devices for Treatment of Vascular Defects”), U.S. patent application 20180206849 (Hewitt et al., Jul. 26, 2018, “Filamentary Devices for the Treatment of Vascular Defects”), U.S. patent application 20210007754 (Milhous et al., Jan. 14, 2021, “Filamentary Devices for Treatment of Vascular Defects”), U.S. patent Ser. No. 10/939,914 (Hewitt et al., Mar. 9, 2021, “Filamentary Devices for the Treatment of Vascular Defects”), and U.S. patent application 20210275184 (Hewitt et al., Sep. 9, 2021, “Filamentary Devices for Treatment of Vascular Defects”) disclose occlusion devices with permeable shells made of woven braided mesh having a variable mesh density and/or porosity. U.S. Pat. No. 9,039,726 (Becking, May 26, 2015, “Filamentary Devices for Treatment of Vascular Defects”) discloses braid-balls for aneurysm occlusion.

U.S. Pat. No. 9,492,174 (Hewitt et al., Nov. 15, 2016, “Filamentary Devices for Treatment of Vascular Defects”), U.S. patent application 20170095254 (Hewitt et al., Apr. 6, 2017, “Filamentary Devices for Treatment of Vascular Defects”), U.S. patent Ser. No. 10/136,896 (Hewitt et al., Nov. 27, 2018, “Filamentary Devices for Treatment of Vascular Defects”), U.S. patent application 20190192166 (Hewitt et al., Jun. 27, 2019, “Filamentary Devices for Treatment of Vascular Defects”), U.S. patent application 20200289124 (Rangwala et al., Sep. 17, 2020, “Filamentary Devices for Treatment of Vascular Defects”), U.S. patent Ser. No. 10/813,645 (Hewitt et al., Oct. 27, 2020, “Filamentary Devices for Treatment of Vascular Defects”), and U.S. patent application 20210106337 (Hewitt et al., Apr. 15, 2021, “Filamentary Devices for Treatment of Vascular Defects”) disclose a self-expanding permeable shell having a radially constrained elongated state configured for delivery within a catheter lumen, an expanded state with a globular and longitudinally shortened configuration relative to the radially constrained state, and a plurality of elongate filaments that are woven together.

U.S. patent application 20180000489 (Marchand et al., Jan. 4, 2018, “Filamentary Devices for Treatment of Vascular Defects”) discloses a self-expanding resilient permeable shell having a plurality of elongate resilient filaments with a woven structure. U.S. patent application 20200289126 (Hewitt et al., Sep. 17, 2020, “Filamentary Devices for Treatment of Vascular Defects”), U.S. patent Ser. No. 11/317,921 (Hewitt et al., May 3, 2022, “Filamentary Devices for Treatment of Vascular Defects”), and U.S. patent application 20220257258 (Hewitt et al., Aug. 18, 2022, “Filamentary Devices for Treatment of Vascular Defects”) disclose a permeable shell or mesh with a stiffer proximal portion at the neck of an aneurysm. U.S. patent application 20220192678 (Hewitt et al., Jun. 23, 2022, “Filamentary Devices for Treatment of Vascular Defects”) discloses an implant having a first permeable shell having a proximal hub and an open distal end and a second permeable shell having a distal hub and an open proximal end. U.S. patent application 20220249098 (Milhous et al., Aug. 11, 2022, “Filamentary Devices for Treatment of Vascular Defects”) discloses a permeable implant with a plurality of scaffolding filaments. U.S. patent application 20220257260 (Hewitt et al., Aug. 18, 2022, “Filamentary Devices for Treatment of Vascular Defects”) discloses an implant having multiple mesh layers.

U.S. patent application 20200289125 (Dholakia et al., Sep. 17, 2020, “Filamentary Devices Having a Flexible Joint for Treatment of Vascular Defects”) discloses an implant with a first permeable shell having a proximal end with a concave or recessed section and a second permeable shell having a convex section that mates with the concave or recessed section. U.S. patent application 20220039804 (Rangwala et al., Feb. 10, 2022, “Flow-Diverting Implant and Delivery Method”) discloses a saddle-shaped braided mesh diverter that covers the neck of an aneurysm.

U.S. patent application 20200113576 (Gorochow et al., Apr. 16, 2020, “Folded Aneurysm Treatment Device and Delivery Method”) and U.S. patent application 20210196284 (Gorochow et al., Jul. 1, 2021, “Folded Aneurysm Treatment Device and Delivery Method”) disclose an implant having a braided section that folds to form an outer occlusive sack extending across a neck of an aneurysm to engage a wall of the aneurysm from within a sac of the aneurysm and an inner occlusive sack forming a trough nested within the outer occlusive sack. The implant can be closed at one or more of the braid ends to define a substantially enclosed bowl-shaped volume. U.S. patent application 20230017150 (Lee et al., Jan. 19, 2023, “Hydrogel Stent and Embolization Device for Cerebral Aneurysm”) discloses a hydrogel stent for occluding a cerebral aneurysm.

U.S. patent application 20210145449 (Gorochow, May 20, 2021, “Implant Delivery System with Braid Cup Formation”) discloses an implant system with an engagement wire, a pull wire, and a braided implant having a distal ring thereon. U.S. patent application 20210186518 (Gorochow et al., Jun. 24, 2021, “Implant Having an Intrasaccular Section and Intravascular Section”) and U.S. patent Ser. No. 11/457,926 (Gorochow et al., Oct. 4, 2022, “Implant Having an Intrasaccular Section and Intravascular Section”) disclose a tubular braid with an intrasaccular section, an intravascular section, a pinched section, and a predetermined shape. U.S. patent application 20210052279 (Porter et al., Feb. 25, 2021, “Intra-Aneurysm Devices”) discloses a device including an upper member that sits against the dome of an aneurysm, a lower member that sits in the neck of the aneurysm, and a means of adjusting the overall dimensions of the device.

U.S. patent application 20210007755 (Lorenzo et al., Jan. 14, 2021, “Intrasaccular Aneurysm Treatment Device with Varying Coatings”) discloses an aneurysm intrasaccular implant with coated regions. U.S. patent application 20200187952 (Walsh et al., Jun. 18, 2020, “Intrasaccular Flow Diverter for Treating Cerebral Aneurysms”) and U.S. patent application 20220151632 (Walsh et al., May 19, 2022, “Intrasaccular Flow Diverter for Treating Cerebral Aneurysms”) disclose a stabilizing frame with two parts, the first part sized to anchor within the sac of the aneurysm and the exterior part sized to anchor against a region of the blood vessel wall adjacent the aneurysm neck.

U.S. patent application 20220087681 (Xu et al., Mar. 24, 2022, “Inverting Braided Aneurysm Implant with Dome Feature”) discloses an implant with a dome feature configured to press into aneurysm walls near the aneurysm's dome and facilitate securement of the braid across the aneurysm's neck. U.S. patent application 20210085333 (Gorochow et al., Mar. 25, 2021, “Inverting Braided Aneurysm Treatment System and Method”), U.S. patent Ser. No. 11/278,292 (Gorochow et al., Mar. 22, 2022, “Inverting Braided Aneurysm Treatment System and Method”), and U.S. patent application 20220104829 (Gorochow et al., Apr. 7, 2022, “Inverting Braided Aneurysm Treatment System and Method”) disclose a tubular braid with an intrasaccular section, an intravascular section, a pinched section, and a predetermined shape.

U.S. patent Ser. No. 10/653,425 (Gorochow et al., May 19, 2020, “Layered Braided Aneurysm Treatment Device”), U.S. patent application 20200367893 (Xu et al., Nov. 26, 2020, “Layered Braided Aneurysm Treatment Device”), U.S. patent application 20200367898 (Gorochow et al., Nov. 26, 2020, “Layered Braided Aneurysm Treatment Device”), U.S. patent Ser. No. 11/413,046 (Xu et al., Aug. 16, 2022, “Layered Braided Aneurysm Treatment Device”), and U.S. patent application 20200367900 (Pedroso et al., Nov. 26, 2020, “Layered Braided Aneurysm Treatment Device With Corrugations”) disclose a tubular braid comprising an open end, a pinched end, and a predetermined shape; wherein, in the predetermined shape, the tubular braid comprises: a first segment extending from the open end to a first inversion, a second segment encircled by the open end such that the second segment is only partially surrounded by the first segment and extending from the first inversion to a second inversion, and a third segment surrounded by the second segment and extending from the second inversion to the pinched end.

U.S. patent application 20120283768 (Cox et al., Nov. 8, 2012, “Method and Apparatus for the Treatment of Large and Giant Vascular Defects”) discloses the deployment of multiple permeable shell devices within a single vascular defect. U.S. patent application 20120165919 (Cox et al., Jun. 28, 2012, “Methods and Devices for Treatment of Vascular Defects”) and U.S. patent application 20140052233 (Cox et al., Feb. 20, 2014, “Methods and Devices for Treatment of Vascular Defects”) disclose an expandable wire body support structure having a low profile radially constrained state, an expanded relaxed state with a substantially spherical or globular configuration having a smooth outer surface, and a porous permeable layer comprising a braided wire occlusive mesh.

U.S. patent application 20210282789 (Vu et al., Sep. 16, 2021, “Multiple Layer Devices for Treatment of Vascular Defects”) discloses a first permeable shell and a second permeable shell, where the second permeable shell sits within an interior cavity of the first permeable shell. U.S. patent application 20160249937 (Marchand et al., Sep. 1, 2016, “Multiple Layer Filamentary Devices for Treatment of Vascular Defects”), U.S. Pat. No. 9,918,720 (Marchand et al., Mar. 20, 2018, “Multiple Layer Filamentary Devices for Treatment of Vascular Defects”), and U.S. patent Ser. No. 10/238,393 (Marchand et al., Mar. 26, 2019, “Multiple Layer Filamentary Devices for Treatment of Vascular Defects”) disclose a permeable shell and an inner structure configured to occlude blood flow. U.S. patent application 20200367904 (Becking et al., Nov. 26, 2020, “Multiple Layer Filamentary Devices for Treatment of Vascular Defects”) and U.S. patent application 20220022886 (Becking et al., Jan. 27, 2022, “Multiple Layer Filamentary Devices for Treatment of Vascular Defects”) disclose braid-balls suitable for aneurysm occlusion.

U.S. patent application 20170156734 (Griffin, Jun. 8, 2017, “Occlusion Device”), U.S. patent Ser. No. 10/285,711 (Griffin, May 14, 2019, “Occlusion Device”), U.S. patent application 20190269414 (Griffin, Sep. 5, 2019, “Occlusion Device”), U.S. patent application 20210153871 (Griffin, May 27, 2021, “Occlusion Device”), and U.S. patent application 20220313274 (Griffin, Oct. 6, 2022, “Occlusion Device”) disclose a continuous compressible mesh structure comprising axial mesh carriages configured end to end, wherein each end of each carriage is a pinch point in the continuous mesh structure. U.S. patent application 20140200607 (Sepetka et al., Jul. 17, 2014, “Occlusive Device”), U.S. patent application 20190274691 (Sepetka et al., Sep. 12, 2019, “Occlusive Device”), and U.S. patent Ser. No. 11/045,203 (Sepetka et al., Jun. 29, 2021, “Occlusive Device”) disclose multiple sequentially deployed occlusive devices that are connected together to create an extended length. U.S. patent application 20210282784 (Sepetka et al., Sep. 16, 2021, “Occlusive Device”) discloses a device comprising a plurality of braided wires and an embolic coil.

U.S. patent application 20170079662 (Rhee et al., Mar. 23, 2017, “Occlusive Devices”) discloses an implant with a frame and a mesh component, wherein the mesh component has a first porosity and the frame has a second porosity. U.S. patent application 20170079661 (Bardsley et al., Mar. 23, 2017, “Occlusive Devices”), U.S. patent Ser. No. 10/314,593 (Bardsley et al., Jun. 11, 2019, “Occlusive Devices”), and U.S. patent application 20190269411 (Bardsley et al., Sep. 5, 2019, “Occlusive Devices”) disclose an implant with a single- or dual-layer braided body with variable porosity. U.S. patent application 20190343532 (Divino et al., Nov. 14, 2019, “Occlusive Devices”) discloses a device with at least one expandable structure which is adapted to transition from a compressed configuration to an expanded configuration when released into an aneurysm.

U.S. patent Ser. No. 10/478,194 (Rhee et al., Nov. 19, 2019, “Occlusive Devices”) and U.S. patent application 20200038032 (Rhee et al., Feb. 6, 2020, “Occlusive Devices”) disclose an implant with a frame and a mesh component, wherein the mesh component has a first porosity and the frame has a second porosity. U.S. patent application 20220202425 (Gorochow et al., Jun. 30, 2022, “Semispherical Braided Aneurysm Treatment System and Method”) discloses a tubular braid with three segments and two inversions, one of the three segments extending between the two inversions and forming a sack.

U.S. patent Ser. No. 11/185,335 (Badruddin et al., Nov. 30, 2021, “System for and Method of Treating Aneurysms”) discloses an apparatus for treating an aneurysm with an occlusion element disposed on a wire, wherein the occlusion element includes a cover for covering a neck of an aneurysm and an inner anchoring member. U.S. patent application 20200367896 (Zaidat et al., Nov. 26, 2020, “Systems and Methods for Treating Aneurysms”) discloses an apparatus for treating an aneurysm in a blood vessel with a first tubular mesh having a first end and a second end coupled together at a proximal end of the occlusion element. U.S. patent application 20210128165 (Pulugurtha et al., May 6, 2021, “Systems and Methods for Treating Aneurysms”) and U.S. patent Ser. No. 11/305,387 (Pulugurtha et al., Apr. 19, 2022, “Systems and Methods for Treating Aneurysms”) disclose a distal conduit coupled to an occlusive member with a first lumen extending therethrough and a proximal conduit with a second lumen extending therethrough.

U.S. patent application 20210128160 (Li et al., May 6, 2021, “Systems and Methods for Treating Aneurysms”), U.S. patent application 20210128167 (Patel et al., May 6, 2021, “Systems and Methods for Treating Aneurysms”), U.S. patent application 20210128168 (Nguyen et al., May 6, 2021, “Systems and Methods for Treating Aneurysms”) and disclose delivering an occlusive member (e.g., an expandable braid) to an aneurysm sac in conjunction with an embolic element (e.g., coils, embolic material). U.S. patent Ser. No. 11/202,636 (Zaidat et al., Dec. 21, 2021, “Systems and Methods for Treating Aneurysms”), U.S. patent application 20220022884 (Wolfe et al., Jan. 27, 2022, “Systems and Methods for Treating Aneurysms”), and U.S. patent application 20220211383 (Pereira et al., Jul. 7, 2022, “Systems and Methods for Treating Aneurysms”) disclose an apparatus for treating an aneurysm including an occlusion element configured to be releasably coupled to an elongate delivery shaft and a distal end, a proximal end, and a longitudinal axis extending between the distal end and the proximal end.

U.S. patent application 20220054141 (Zaidat et al., Feb. 24, 2022, “Systems and Methods for Treating Aneurysms”) discloses an apparatus for treating an aneurysm in a blood vessel with a first tubular mesh having a first end and a second end coupled together at a proximal end of the occlusion element. U.S. patent Ser. No. 10/398,441 (Warner et al., Sep. 3, 2019, “Vascular Occlusion”) discloses a vascular treatment system with a containment device, a pusher, and a stopper ring. U.S. patent application 20200038034 (Maguire et al., Feb. 6, 2020, “Vessel Occluder”) discloses a vessel occluder with an expandable mesh portion having a flexible membrane that expands within a cavity of the expandable mesh portion.

### SUMMARY OF THE INVENTION

This invention is an intrasacular aneurysm occlusion device with a proximal bowl-shaped mesh (or net) which covers the neck of an aneurysm sac and a distal globular mesh (or net) between the proximal bowl-shaped mesh and a distal dome of the aneurysm sac. The proximal bowl-shaped mesh blocks blood flow into the aneurysm sac. The distal globular mesh holds the bowl-shaped mesh in place against the aneurysm neck. In an example, the proximal bowl-shaped mesh can have a hemispherical or half-ellipsoidal shape. In an example, the distal globular mesh can be have a spherical, ellipsoidal, or cardioid shape. In an example, the proximal bowl-shaped mesh and the distal globular mesh can be formed from the same continuous mesh.

In an example, the proximal bowl-shaped mesh and the distal globular mesh can be formed from a tubular mesh by: (a) forming two globular meshes from the tubular mesh by radially-constraining the distal end of the tubular mesh, radially-constraining a middle portion of the tubular mesh, and radially-constraining the proximal end of the tubular mesh; (b) longitudinally-stretching and radially-compressing the two globular meshes for delivery through a catheter to an aneurysm sac; (c) inserting and radially-expanding the two globular meshes within the aneurysm sac; and then (d) forming a proximal bowl-shaped mesh and a distal globular mesh within the aneurysm sac by compressing and/or folding the proximal globular mesh into bowl-shaped mesh, wherein the distal globular mesh is nested within the concavity of the proximal bowl-shaped mesh.

### BRIEF INTRODUCTION TO THE FIGURES

FIG. 1 shows a taxonomy of intrasacular aneurysm occlusion devices having a proximal bowl-shaped mesh (e.g. a “bowl”) and a distal globular mesh (e.g. a “ball”).

FIG. 2 shows an aneurysm occlusion device wherein the ball and bowl are not continuous and wherein the ball does not have a distal inversion.

FIG. 3 shows an aneurysm occlusion device wherein the ball and bowl are not continuous and wherein the ball has a distal inversion.

FIG. 4 shows an aneurysm occlusion device wherein the ball and bowl are continuous and share some perimeter and wherein the ball does not have a distal inversion.

FIG. 5 shows an aneurysm occlusion device wherein the ball and bowl are continuous and share some perimeter and wherein the ball has a distal inversion.

FIG. 6 shows an aneurysm occlusion device wherein the ball and bowl are continuous, the bowl has been formed by proximal inversion, and the ball does not have a distal inversion.

FIG. 7 shows an aneurysm occlusion device wherein the ball and bowl are continuous, the bowl has been formed by proximal inversion, and the ball has a distal inversion.

FIG. 8 shows an aneurysm occlusion device wherein the ball and bowl are continuous, the bowl has been formed by proximal inversion, the bowl has two layers, and the ball has a distal inversion.

FIG. 9 through 11 show three sequential views of an aneurysm occlusion device with a distal stent and a proximal stent. FIG. 9 shows this device when the distal and proximal stents are compressed within a catheter. FIG. 10 shows this device when the distal stent has exited the catheter, but the proximal stent has not yet exited the catheter. FIG. 11 shows this device when both the distal and proximal stents have expanded within the aneurysm sac.

FIG. 12 shows an aneurysm occlusion device with a proximal bowl-shaped mesh or net and a distal sac-filling mesh or net.

FIG. 13 shows an aneurysm occlusion device with a proximal ball-shaped mesh or net and a distal sac-filling mesh or net.

FIG. 14 shows an aneurysm occlusion device with a proximal bowl-shaped mesh and a distal globular mesh nested in the proximal bowl-shaped mesh.

FIG. 15 shows an aneurysm occlusion device with a mesh or net which is filled with longitudinal strands of embolic members (e.g. “string of pearls” embolic strands).

FIG. 16 shows an aneurysm occlusion device with a proximal bowl-shaped (e.g. hemispherical) mesh or net and a distal globular mesh or net, wherein the distal globular mesh or net is filled with longitudinal strands of embolic members (e.g. “string of pearls” embolic strands).

FIG. 17 shows an aneurysm occlusion device with a proximal bowl-shaped (e.g. half-torus) mesh or net and a distal globular mesh or net, wherein the distal globular mesh or net is filled with longitudinal strands of embolic members (e.g. “string of pearls” embolic strands).

FIG. 18 shows an aneurysm occlusion device with a proximal bowl-shaped mesh and a distal globular mesh, wherein the globular mesh is nested within the bowl-shaped mesh.

FIG. 19 shows an aneurysm occlusion device with a proximal bowl-shaped mesh or net, a distal globular mesh or net, and a valve through which embolic members are inserted into the globular mesh or net.

FIG. 20 through 23 show four sequential views of the formation and deployment of an aneurysm occlusion device with a single-layer proximal bowl-shaped mesh and a distal globular mesh, wherein the bowl-shaped mesh is formed by radially-constraining and everting a tubular mesh. FIG. 20 introduces the tubular mesh. FIG. 21 shows how the proximal bowl-shaped mesh and the distal globular mesh are made by radially-constraining, inverting, and everting the tubular mesh. FIG. 22 shows this device after it has been inserted into an aneurysm sac, wherein embolic material is starting to be delivered through into the aneurysm sac. FIG. 23 shows the device after the aneurysm sac has been filed with embolic material and the catheter has been removed.

FIG. 24 shows an aneurysm occlusion device with a bowl-shaped mesh inside a globular mesh.

FIG. 25 shows two sequential views of an aneurysm occlusion device with a bowl-shaped mesh inside a globular mesh, wherein embolic material is being inserted into the globular mesh.

FIG. 26 shows an aneurysm occlusion device with a disk-shaped mesh inside a globular mesh.

FIG. 27 shows an aneurysm occlusion device with a globular mesh nested in a bowl-shaped mesh.

FIG. 28 shows an aneurysm occlusion device with a distal bowl-shaped mesh inside a proximal bowl-shaped mesh.

FIG. 29 shows an aneurysm occlusion device with an inverted jug or inverted-bottle shaped mesh nested in a bowl-shaped mesh.

FIG. 30 through 34 show four sequential views of the formation and deployment of an intrasacular aneurysm occlusion device with a proximal bowl-shaped mesh and a distal globular mesh which is formed from a tubular mesh. FIG. 30 introduces the tubular mesh. FIG. 31 shows the mesh having been inverted and radially-constrained at multiple longitudinal locations to form two globular meshes. FIG. 32 shows the two globular meshes having been radially-compressed and elongated for delivery through a catheter. FIG. 33 shows the device having been inserted into an aneurysm sac. FIG. 34 shows the proximal globular mesh having been compressed or folded to form a proximal bowl-shaped mesh into which the distal globular mesh is nested.

FIG. 35 shows an aneurysm occlusion device comprising an intrasacular arcuate distal stent and an intrasacular arcuate proximal stent, wherein the proximal stent has a concavity into which a portion of the distal stent fits when the device is deployed within an aneurysm sac.

### DETAILED DESCRIPTION OF THE FIGURES

In an example, an intrasacular aneurysm occlusion device can comprise: a proximal bowl-shaped mesh or net which is configured to be inserted into an aneurysm sac and expanded to span a neck of the aneurysm sac; and a distal globular mesh or net which is configured to be inserted into the aneurysm sac between the proximal bowl-shaped mesh or net and a distal dome of the aneurysm sac to hold the proximal bowl-shaped mesh or net against the neck of the aneurysm.

In an example, a proximal bowl-shaped mesh or net can have a hemispherical shape. In an example, a proximal bowl-shaped mesh or net can have a hemi-ellipsoidal shape. In an example, a proximal bowl-shaped mesh or net can have a convex lens shape. In an example, a proximal bowl-shaped mesh or net can have a cup shape. In an example, a proximal portion of a proximal bowl-shaped mesh or net can be thicker than a distal portion of a proximal bowl-shaped mesh or net. In an example, a central portion of a proximal bowl-shaped mesh or net can be thicker than a peripheral portion of a proximal bowl-shaped mesh or net.

In an example, a proximal bowl-shaped mesh or net can have a shape selected from the group consisting of: convex, bowl shaped, funnel, hemispherical, paraboloid of revolution, conic section, and hyperbolic. In an example, a proximal bowl-shaped mesh or net can have a post-expansion shape which is selected from the group consisting of: convex lens shape, bowl shaped, funnel, hemispherical, paraboloid of revolution, conic section, section of an ellipsoid, and hyperbolic.

In an example, the size of a proximal bowl-shaped mesh or net can be remotely adjusted by the operator of the device after the mesh or net has been inserted into an aneurysm sac. In an example, the width of a proximal bowl-shaped mesh or net can be remotely adjusted by the operator of the device after the mesh or net has been inserted into an aneurysm sac. In an example, the height of a proximal bowl-shaped mesh or net can be remotely adjusted by the operator of the device after the mesh or net has been inserted into an aneurysm sac. In an example, the shape of a proximal bowl-shaped mesh or net can be remotely adjusted by the operator of the device after the mesh or net has been inserted into an aneurysm sac.

In an example, the size of a proximal bowl-shaped mesh or net can be remotely adjusted by the application of electrical energy to selected portions of the mesh or net after the mesh or net has been inserted into an aneurysm sac. In an example, the width of a proximal bowl-shaped mesh or net can be remotely adjusted by the application of electrical energy to selected portions of the mesh or net after the mesh or net has been inserted into an aneurysm sac. In an example, the height of a proximal bowl-shaped mesh or net can be remotely adjusted by the application of electrical energy to selected portions of the mesh or net after the mesh or net has been inserted into an aneurysm sac. In an example, the shape of a proximal bowl-shaped mesh or net can be remotely adjusted by the application of electrical energy to selected portions of the mesh or net after the mesh or net has been inserted into an aneurysm sac.

In an example, there can be a lateral cross-section of a proximal bowl-shaped mesh or net, wherein this cross-section is perpendicular to the proximal-to-distal axis of the mesh or net, and wherein this cross-section has a circular shape. In an example, there can be a lateral cross-section of a proximal bowl-shaped mesh or net, wherein this cross-section is perpendicular to the proximal-to-distal axis of the mesh or net, and wherein this cross-section has an elliptical shape. In an example, there can be a lateral cross-section of a proximal bowl-shaped mesh or net, wherein this cross-section is perpendicular to the proximal-to-distal axis of the mesh or net, and wherein this cross-section has an undulating, wavy, or sinusoidal shape.

In an example, there can be a longitudinal cross-section of a proximal bowl-shaped mesh or net, wherein this cross-section is parallel to the proximal-to-distal axis of the mesh or net, and wherein this cross-section has a semi-circular shape. In an example, there can be a longitudinal cross-section of a proximal bowl-shaped mesh or net, wherein this cross-section is parallel to the proximal-to-distal axis of the mesh or net, and wherein this cross-section has an undulating, wavy, or sinusoidal shape.

In an example, a proximal bowl-shaped mesh or net can be a mesh. In an example, a proximal bowl-shaped mesh or net can be a braided mesh. In an example, a proximal bowl-shaped mesh or net can be a woven mesh. In an example, a proximal bowl-shaped mesh or net can be a 3D-printed mesh. In an example, a proximal bowl-shaped mesh or net can be a mesh which is made by using a laser to make holes in a membrane or sheet of material. In an example, a proximal bowl-shaped mesh or net can be a net. In an example, a proximal bowl-shaped mesh or net can be a braided net. In an example, a proximal bowl-shaped mesh or net can be a woven net. In an example, a proximal bowl-shaped mesh or net can be a 3D-printed net. In an example, a proximal bowl-shaped mesh or net can be a net which is made by using a laser to make holes in a membrane or sheet of material.

In an example, a proximal bowl-shaped mesh or net can be made by braiding. In an example, a proximal bowl-shaped mesh or net can be made by braiding wires, tubes, and/or filaments. In an example, a proximal bowl-shaped mesh or net can be made by weaving. In an example, a proximal bowl-shaped mesh or net can be made by weaving wires, tubes, and/or filaments. In an example, a proximal bowl-shaped mesh or net can be made by 3D printing. In an example, a proximal bowl-shaped mesh or net can be made by 3D printing wires, tubes, and/or filaments. In an example, a proximal bowl-shaped mesh or net can comprise a metal mesh or net. In an example, a proximal bowl-shaped mesh or net can comprise a polymer mesh or net. In an example, a proximal bowl-shaped mesh or net can comprise be a stent. In an example, a proximal bowl-shaped mesh or net can be made with shape-memory material.

In an example, a proximal bowl-shaped mesh or net can be braided or woven from longitudinal wires, tubes, or filaments with longitudinal orientations which are substantially parallel with a proximal-to-distal axis of the mesh or net. In an example, a proximal bowl-shaped mesh or net can be braided or woven from longitudinal wires, tubes, or filaments with longitudinal orientations which are substantially perpendicular to a proximal-to-distal axis of the mesh or net. In an example, a proximal bowl-shaped mesh or net can be braided or woven from longitudinal wires, tubes, or filaments with longitudinal orientations which are substantially parallel with a circumference of the mesh or net. In an example, a proximal bowl-shaped mesh or net can be braided or woven from longitudinal wires, tubes, or filaments with longitudinal orientations which are substantially concentric with a circumference of the mesh or net.

In an example, a proximal bowl-shaped mesh or net can be braided or woven from helical longitudinal wires, tubes, or filaments. In an example, a proximal bowl-shaped mesh or net can be braided or woven from: a first set of wires, tubes, or filaments which are substantially parallel with a proximal-to-distal axis of the mesh or net; and a second set of wires, tubes, or filaments which are substantially perpendicular to a proximal-to-distal axis of the mesh or net. In an example, a proximal bowl-shaped mesh or net can be braided or woven from: a first set of wires, tubes, or filaments which are substantially parallel with a proximal-to-distal axis of the mesh or net; and a second set of wires, tubes, or filaments which are substantially concentric with a circumference of the mesh or net.

In an example, a proximal bowl-shaped mesh or net can be braided or woven from wires, tubes, or filaments which extend radially outward from the center of the bowl-shaped mesh or net. In an example, a proximal bowl-shaped mesh or net can be braided or woven from: a first set of wires, tubes, or filaments which extend radially outward from the center of the bowl-shaped mesh or net; and a second set of wires, tubes, or filaments which are substantially concentric with a circumference of the mesh or net. In an example, a proximal bowl-shaped mesh or net can be braided or woven from: a first set of wires, tubes, or filaments which extend radially outward from the center of the bowl-shaped mesh or net; and a second set of wires, tubes, or filaments which are helical.

In an example, a proximal bowl-shaped mesh or net can be braided or woven from undulating, wavy, and/or sinusoidal wires, tubes, or filaments. In an example, a proximal bowl-shaped mesh or net can be braided or woven from: a first set of wires, tubes, or filaments which are undulating, wavy, and/or sinusoidal; and a second set of wires, tubes, or filaments which are not undulating, wavy, and/or sinusoidal. In an example, a proximal bowl-shaped mesh or net can comprise a braided or woven combination of undulating and helical wires, tubes, or filaments.

In an example, a proximal bowl-shaped mesh or net can have circular holes (e.g. holes, openings, or pores). In an example, a proximal bowl-shaped mesh or net can have hexagonal holes (e.g. holes, openings, or pores). In an example, a proximal bowl-shaped mesh or net can have triangular holes (e.g. holes, openings, or pores). In an example, a proximal bowl-shaped mesh or net can have quadrilateral holes (e.g. holes, openings, or pores). In an example, a proximal bowl-shaped mesh or net can have holes (e.g. holes, openings, or pores) which are smaller than embolic members which are inserted into the aneurysm sac. In an example, a proximal bowl-shaped mesh or net can have holes (e.g. holes, openings, or pores) which are less than half of the size of embolic members which are inserted into the aneurysm sac.

In an example, a proximal bowl-shaped mesh or net can have a resilient circumferential ring or band. In an example, a proximal bowl-shaped mesh or net can have a resilient ring or band around its distal circumference. In an example, a proximal bowl-shaped mesh or net can have a resilient ring or band around its distal circumference, wherein this ring or band is more resilient (e.g. less flexible or lower durometer) than the rest of the mesh or net. In an example, one or more (circular) elastic bands or rings can be woven or braided into a distal globular mesh or net.

In an example, the proximal-to-distal height of a proximal bowl-shaped mesh or net (e.g. the longitudinal axis of the mesh or net) can be a first distance and the side-to-side width of the mesh or net (e.g. the lateral diameters of the mesh or net) can be a second distance, wherein the second distance is greater than the first distance. In an example, the proximal-to-distal height of a proximal bowl-shaped mesh or net (e.g. the longitudinal axis of the mesh or net) can be a first distance and the side-to-side width of the mesh or net (e.g. the lateral diameters of the mesh or net) can be a second distance, wherein the second distance is twice the first distance. In an example, the proximal-to-distal height of a proximal bowl-shaped mesh or net (e.g. the longitudinal axis of the mesh or net) can be a first distance and the side-to-side width of the mesh or net (e.g. the lateral diameters of the mesh or net) can be a second distance, wherein the second distance is between 50% and 100% greater than the first distance.

In an example, a proximal bowl-shaped mesh or net can have a single layer. In an example, a proximal bowl-shaped mesh or net can have two layers. In an example, a proximal bowl-shaped mesh or net can have a distal layer which faces toward the aneurysm dome and a proximal layer which faces toward the aneurysm neck. In an example, a proximal bowl-shaped mesh or net can have a hemispherical-shaped distal surface. In an example, a proximal bowl-shaped mesh or net can have a hemispherical-shaped proximal surface.

In an example, a proximal bowl-shaped mesh or net can have two layers. In an example, a proximal bowl-shaped mesh or net can have a proximal layer and a distal layer. In an example, a proximal bowl-shaped mesh or net can have a proximal layer and a distal layer which connect to each other around the distal circumference of the proximal bowl-shaped mesh or net. In an example, a proximal bowl-shaped mesh or net can have a proximal layer with a proximal-facing concavity and a distal layer with a distal-facing concavity. In an example, a proximal bowl-shaped mesh or net can have a proximally-concave proximal layer and a distally-concave distal layer with a distal-facing concavity.

In an example, a proximal bowl-shaped mesh or net can have a proximal polymer mesh layer and a distal metal mesh layer. In an example, a proximal bowl-shaped mesh or net can have a proximal layer, a distal layer, and a middle layer between the proximal and distal layers. In an example, the proximal and distal layers can be metal meshes or nets and the middle layer can be a polymer mesh or net. In an example, a proximal bowl-shaped mesh or net can comprise a metal mesh or net which has been coated with a polymer material. In an example, a proximal bowl-shaped mesh or net can comprise a metal mesh or net into which has been woven a polymer material. In an example, a proximal bowl-shaped mesh or net can comprise a metal mesh or net which has been coated with a hydrogel material.

In an example, a proximal bowl-shaped mesh or net can have two layers, a proximal layer and a distal layer, wherein the distance between these two layers is uniform. In an example, a proximal bowl-shaped mesh or net can have two layers, a proximal layer and a distal layer, wherein the distance between is greater in the central area of the bowl and less in the peripheral areas of the bowl. In an example, a proximal bowl-shaped mesh or net can have two layers, a proximal layer and a distal layer, wherein the proximal layer is uniformly concave, but the distal layer has locally-concave and locally-convex portions. In an example, a proximal bowl-shaped mesh or net can have two layers, a proximal layer and a distal layer, wherein the proximal layer is more dense, less porous, thicker, and/or less elastic than the distal layer. In an example, a proximal bowl-shaped mesh or net can have two layers, a proximal layer and a distal layer, wherein the proximal layer is less dense, more porous, thinner, and/or more elastic than the distal layer.

In an example, the distance between proximal layer or surface of a proximal bowl-shaped mesh or net and the distal layer or surface of the mesh or net can be non-uniform. In an example, the distance between proximal layer or surface of a proximal bowl-shaped mesh or net and the distal layer or surface of the mesh or net can be greater in a central portion of the mesh or net than in a peripheral portion of the mesh or net. In an example, the distance between proximal layer or surface of a proximal bowl-shaped mesh or net and the distal layer or surface of the mesh or net can be less in a central portion of the mesh or net than in a peripheral portion of the mesh or net

In an example, a proximal bowl-shaped mesh or net with two layers can be made by folding, inverting, or everting a mesh or net over itself. In an example, the distal circumference of the flexible mesh or net can be a fold in the mesh or net. In an example, a tubular mesh can be transformed into a single-layer, distally-concave, bowl-shaped flexible mesh or net by a single radially-constraining member (e.g. band or ring) which radially-constrains the proximal end of the tubular mesh. In an example, both the distal end of a tubular mesh and the proximal end of a tubular mesh can be radially-constrained by a proximal radially-constraining member (e.g. band or ring) to form a two-layer bowl-shaped flexible mesh or net which is inserted into an aneurysm sac.

In an example, a tubular mesh can be transformed into a double-layer, distally-concave, bowl-shaped flexible mesh or net by a single radially-constraining member (e.g. band or ring) in a middle section (between the ends) of a tubular mesh which radially-constrains the middle of the tubular mesh, wherein the proximal portion of the mesh is everted distally over the distal portion of the mesh until it has a distally-concave shape. In an example, a tubular mesh can be transformed into a double-layer, distally-concave, bowl-shaped flexible mesh or net by two radially-constraining members (e.g. bands or rings) (a proximal radially-constraining member (e.g. band or ring) and a distal radially-constraining member (e.g. band or ring) which radially-constrain the proximal and distal ends of the tubular mesh, wherein the distal portion of the tubular mesh is inverted proximally (e.g. folded proximally) until it has a distally-concave shape.

In an example, a proximal bowl-shaped mesh or net can be made using 3D printing. In an example, a proximal bowl-shaped mesh or net can be made by 3D printing with a polymer material. In an example, a proximal bowl-shaped mesh or net can be made by 3D printing with a silicone-based polymer. In an example, a proximal bowl-shaped mesh or net can be made by 3D printing with an elastomeric polymer. In an example, a proximal bowl-shaped mesh or net can be made by 3D printing with polydimethylsiloxane (PDMS). In an example, a proximal bowl-shaped mesh or net can be made by 3D printing with liquid metal. In an example, a proximal bowl-shaped mesh or net can be made by using a laser to make holes in a metal film and/or sheet. In an example, a proximal bowl-shaped mesh or net can be made by using a laser to make holes in a polymer film and/or sheet.

In an example, a two-layer proximal bowl-shaped mesh or net can be created by compressing, inverting, and/or folding a globular mesh or net. In an example, a two-layer proximal bowl-shaped mesh or net can be created by compressing, inverting, and/or folding the distal half of a globular mesh or net into the proximal half of the globular mesh or net. In an example, a two-layer proximal bowl-shaped mesh or net can be created by compressing, inverting, and/or folding the distal hemisphere of a globular mesh or net into the proximal hemisphere of the globular mesh or net. In an example, a two-layer proximal bowl-shaped mesh or net can be created by compressing, inverting, and/or folding the distal half of a globular mesh or net so that it is nested with the proximal half of the globular mesh or net. In an example, a two-layer proximal bowl-shaped mesh or net can be created by compressing, inverting, and/or folding the distal hemisphere of a globular mesh or net so that it is nested within the proximal hemisphere of the globular mesh or net. In an example, accumulation of embolic members or material in an aneurysm sac can compress, invert, and/or fold a single-layer globular mesh into a two-layer bowl-shaped mesh.

In an example, a proximal bowl-shaped mesh or net can self-expand into a bowl shape within an aneurysm sac in a single-step transition, from its first (constrained) configuration during delivery to its second (expanded) configuration within the sac. In an example, a proximal bowl-shaped mesh or net can be self-expanded into a bowl shape in a multi-step transition from its first (constrained) configuration to its second (expanded) configuration. In an example, a proximal bowl-shaped mesh or net can be created in two steps by: first, expanding a single-layer spherical or ellipsoidal mesh or net; and, second, compressing, collapsing, inverting, and/or folding this spherical or ellipsoidal mesh or net into two-layer bowl-shaped mesh or net. In an example, the second step can be executing by the device operator by: pulling a wire, cord, string, or cable which is connected to the mesh or net; rotating a wire which is connected to the mesh or net; inserted embolic material into the aneurysm sac distal to the mesh or net; or applying electrical current to the mesh or net.

In an example, a proximal bowl-shaped mesh or net can be made from a spherical or ellipsoidal mesh or net which has been compressed, folded, or inverted along its central circumference to create a double-layer bowl-shaped mesh or net. In an example, a proximal bowl-shaped mesh or net can be made from a spherical or ellipsoidal mesh or net which has been compressed, folded, or inverted along its central circumference to create a double-layer bowl-shaped mesh or net before it is inserted into an aneurysm sac. In an example, a proximal bowl-shaped mesh or net can be made from a spherical or ellipsoidal mesh or net which has been inserted into an aneurysm sac and then compressed, folded, or inverted along its central circumference to create a double-layer bowl-shaped mesh or net.

In an example, a proximal bowl-shaped mesh or net can be formed by: radially-constraining a tubular mesh or net at two different points along the longitudinal axis of the tubular mesh or net; and then moving the two different points closer to each other. In an example, a proximal bowl-shaped mesh or net can be formed by radially-constraining the ends of a tubular mesh or net and moving the two ends close to each other. In an example, a proximal bowl-shaped mesh or net can be formed by: radially-constraining a tubular mesh or net at two different points along the longitudinal axis of the tubular mesh or net; and moving the two different points closer to each other so as to invert a portion of the tubular mesh.

In an example, a proximal bowl-shaped mesh or net can be made from a tubular mesh or net. In an example, a proximal bowl-shaped mesh or net can be made by inverting or everting a tubular mesh or net into a globular mesh or net and then compressing (e.g. compressing, folding, or inverting) the globular mesh or net. In an example, a proximal bowl-shaped mesh or net can be made by inverting or everting the ends of tubular mesh or net to form a globular mesh or net and then compressing (e.g. compressing, folding, or inverting) the globular mesh or net. In an example, a proximal bowl-shaped mesh or net can be made by radially-constraining and then inverting (or everting) a tubular mesh or net and then compressing (e.g. compressing, folding, or inverting) the globular mesh or net.

In an example, a proximal bowl-shaped mesh or net can be made by radially-constraining and then inverting (or everting) a tubular mesh or net at two locations along the longitudinal axis of the tubular mesh or net and then compressing (e.g. compressing, folding, or inverting) the globular mesh or net. In an example, a proximal bowl-shaped mesh or net can be made by inverting or everting a tubular mesh or net into a globular mesh or net and then compressing the globular mesh or net around its circumference. In an example, a proximal bowl-shaped mesh or net can be made by inverting or everting a tubular mesh or net into a globular mesh or net and then folding the globular mesh or net over its circumference. In an example, a proximal bowl-shaped mesh or net can be made by inverting or everting a tubular mesh or net into a globular mesh or net and then inverting the globular mesh or net around its circumference.

In an example, a proximal bowl-shaped mesh or net can be made by 3D printing. In an example, a proximal bowl-shaped mesh or net can be made by 3D printing a polymer into a porous bowl-shaped structure. In an example, a proximal bowl-shaped mesh or net can be made by creating holes in a bowl-shaped membrane. In an example, a proximal bowl-shaped mesh or net can be made by using a laser to create holes in a bowl-shaped membrane. In an example, a proximal bowl-shaped mesh or net can be made by using a laser to create holes in a bowl-shaped polymer structure.

In an example, a proximal bowl-shaped mesh or net can have uniform density, flexibility, porosity, thickness, and durometer level. In an example, a central portion of a proximal bowl-shaped mesh or net can have a different density, flexibility, porosity, thickness, and/or durometer level than its peripheral portions. In an example, a central portion of a proximal bowl-shaped mesh or net can have a greater density, flexibility, porosity, thickness, and/or durometer level than its peripheral distal portions. In an example, a central portion of a proximal bowl-shaped mesh or net can have a lower density, flexibility, porosity, thickness, and/or durometer level than its peripheral portions.

In an example, a central portion of a proximal bowl-shaped mesh or net and a non-central portion of this proximal bowl-shaped mesh or net can have the same levels of porosity, thickness, elasticity, flexibility, durometer, density, compliance, conformability, and/or malleability. In an example, a central portion of a proximal bowl-shaped mesh or net and a non-central portion of this proximal bowl-shaped mesh or net can have a different levels of porosity, thickness, elasticity, flexibility, durometer, density, compliance, conformability, and/or malleability. In an example, a central portion of a proximal bowl-shaped mesh or net can have a first level of porosity, thickness, elasticity, flexibility, durometer, density, compliance, conformability, and/or malleability; and a non-central portion of this proximal bowl-shaped mesh or net can have a second level of porosity, thickness, elasticity, flexibility, durometer, density, compliance, conformability, and/or malleability; wherein the first level is greater than the second level.

In an example, a central portion of a proximal bowl-shaped mesh or net can be more compliant than a non-central portion of this proximal bowl-shaped mesh or net. In an example, a central portion of a proximal bowl-shaped mesh or net can be less elastic than a non-central portion of this proximal bowl-shaped mesh or net. In an example, a central portion of a proximal bowl-shaped mesh or net can be more conformable than a non-central portion of this proximal bowl-shaped mesh or net. In an example, a central portion of a proximal bowl-shaped mesh or net can be more elastic than a non-central portion of this proximal bowl-shaped mesh or net. In an example, a central portion of a proximal bowl-shaped mesh or net can be more flexible than a non-central portion of this proximal bowl-shaped mesh or net. In an example, a central portion of a proximal bowl-shaped mesh or net can be made from a metal and a non-central portion of this proximal bowl-shaped mesh or net can be made from a polymer.

In an example, a central portion of a proximal bowl-shaped mesh or net can be more malleable than a non-central portion of this proximal bowl-shaped mesh or net. In an example, a central portion of a proximal bowl-shaped mesh or net can be more porous than a non-central portion of this proximal bowl-shaped mesh or net. In an example, a central portion of a proximal bowl-shaped mesh or net can be thicker than a non-central portion of this proximal bowl-shaped mesh or net. In an example, a central portion of a proximal bowl-shaped mesh or net can have a larger hole size than that of a non-central portion of this proximal bowl-shaped mesh or net. In an example, a central portion of a proximal bowl-shaped mesh or net can have a higher durometer level than a non-central portion of this proximal bowl-shaped mesh or net. In an example, a central portion of a proximal bowl-shaped mesh or net can be made from a polymer and a non-central portion of this proximal bowl-shaped mesh or net can be made from a metal.

In an example, a central portion of a proximal bowl-shaped mesh or net can be made with a greater percentage of polymer material than a non-central portion of this proximal bowl-shaped mesh or net. In an example, a central portion of a proximal bowl-shaped mesh or net can be less compliant than a non-central portion of this proximal bowl-shaped mesh or net. In an example, a central portion of a proximal bowl-shaped mesh or net can have a lower durometer level than a non-central portion of this proximal bowl-shaped mesh or net. In an example, a central portion of a proximal bowl-shaped mesh or net can be less flexible than a non-central portion of this proximal bowl-shaped mesh or net. In an example, a central portion of a proximal bowl-shaped mesh or net can be made with a lower percentage of polymer material than a non-central portion of this proximal bowl-shaped mesh or net.

In an example, a central portion of a proximal bowl-shaped mesh or net can be less malleable than a non-central portion of this proximal bowl-shaped mesh or net. In an example, a central portion of a proximal bowl-shaped mesh or net can be made from a different material than a non-central portion of this proximal bowl-shaped mesh or net. In an example, a central portion of a proximal bowl-shaped mesh or net can have a first level of porosity, thickness, elasticity, flexibility, durometer, density, compliance, conformability, and/or malleability; and a non-central portion of this proximal bowl-shaped mesh or net can have a second level of porosity, thickness, elasticity, flexibility, durometer, density, compliance, conformability, and/or malleability; wherein the first level is less than the second level.

In an example, a central portion of a proximal bowl-shaped mesh or net can be less conformable than a non-central portion of this proximal bowl-shaped mesh or net. In an example, a central portion of a proximal bowl-shaped mesh or net can have a smaller hole size than that of distal globular mesh or net. In an example, a central portion of a proximal bowl-shaped mesh or net can have a different hole shape than a non-central portion of this proximal bowl-shaped mesh or net. In an example, a central portion of a proximal bowl-shaped mesh or net can be less porous than a non-central portion of this proximal bowl-shaped mesh or net. In an example, a central portion of a proximal bowl-shaped mesh or net can be thinner than a non-central portion of this proximal bowl-shaped mesh or net.

In an example, a proximal portion of a proximal bowl-shaped mesh or net can have a different density, flexibility, porosity, thickness, and/or durometer level than its distal portion. In an example, a proximal portion of a proximal bowl-shaped mesh or net can have a greater density, flexibility, porosity, thickness, and/or durometer level than its distal portion. In an example, a proximal portion of a proximal bowl-shaped mesh or net can have a lower density, flexibility, porosity, thickness, and/or durometer level than its distal portion. In an example, a proximal half of a proximal bowl-shaped mesh or net can have a different density, flexibility, porosity, thickness, and/or durometer level than its distal half. In an example, a proximal half of a proximal bowl-shaped mesh or net can have a greater density, flexibility, porosity, thickness, and/or durometer level than its distal half. In an example, a proximal half of a proximal bowl-shaped mesh or net can have a lower density, flexibility, porosity, thickness, and/or durometer level than its distal half.

In an example, a central portion of a proximal bowl-shaped mesh or net can be more dense than a peripheral portion of a proximal bowl-shaped mesh or net. In an example, a central portion of a proximal bowl-shaped mesh or net can have a higher durometer level than a peripheral portion of a proximal bowl-shaped mesh or net. In an example, a central portion of a proximal bowl-shaped mesh or net can be less porous than a peripheral portion of a proximal bowl-shaped mesh or net. In an example, a central portion of a proximal bowl-shaped mesh or net can be less elastic than a peripheral portion of the proximal bowl-shaped mesh or net. In an example, wires, tubes, and/or filaments in a central portion of a proximal bowl-shaped mesh or net can thicker than those in a peripheral portion of the proximal bowl-shaped mesh or net.

In an example, a central portion of a proximal bowl-shaped mesh or net can be less dense than a peripheral portion of a proximal bowl-shaped mesh or net. In an example, a central portion of a proximal bowl-shaped mesh or net can have a lower durometer level than a peripheral portion of a proximal bowl-shaped mesh or net. In an example, a central portion of a proximal bowl-shaped mesh or net can be more porous than a peripheral portion of a proximal bowl-shaped mesh or net. In an example, a central portion of a proximal bowl-shaped mesh or net can be more elastic than a peripheral portion of a proximal bowl-shaped mesh or net. In an example, wires, tubes, and/or filaments in a central portion of a proximal bowl-shaped mesh or net can thinner than those in a peripheral portion of the proximal bowl-shaped mesh or net.

In an example, a proximal portion of a proximal bowl-shaped mesh or net can be more dense than a distal portion of a proximal bowl-shaped mesh or net. In an example, a proximal portion of a proximal bowl-shaped mesh or net can have a higher durometer level than a distal portion of a proximal bowl-shaped mesh or net. In an example, a proximal portion of a proximal bowl-shaped mesh or net can be less porous than a distal portion of a proximal bowl-shaped mesh or net. In an example, a proximal portion of a proximal bowl-shaped mesh or net can be less elastic than a distal portion of a proximal bowl-shaped mesh or net. In an example, wires, tubes, and/or filaments in a proximal portion of a proximal bowl-shaped mesh or net can thicker than those in a distal portion of the proximal bowl-shaped mesh or net.

In an example, a proximal portion of a proximal bowl-shaped mesh or net can be less dense than a distal portion of a proximal bowl-shaped mesh or net. In an example, a proximal portion of a proximal bowl-shaped mesh or net can have a lower durometer level than a distal portion of a proximal bowl-shaped mesh or net. In an example, a proximal portion of a proximal bowl-shaped mesh or net can be more porous than a distal portion of a proximal bowl-shaped mesh or net. In an example, a proximal portion of a proximal bowl-shaped mesh or net can be more elastic than a distal portion of a proximal bowl-shaped mesh or net. In an example, wires, tubes, and/or filaments in a proximal portion of a proximal bowl-shaped mesh or net can be thinner than those in a distal portion of the proximal bowl-shaped mesh or net.

In an example, a proximal bowl-shaped mesh or net can be made from a metal. In an example, a proximal bowl-shaped mesh or net can be made from cobalt chromium alloy, nickel-titanium alloy, Nitinol, platinum, tantalum, and/or stainless steel. In an example, a proximal bowl-shaped mesh or net can be made from a polymer. In an example, a proximal bowl-shaped mesh or net can be made from polyethylene terephthalate (PET), polydimethylsiloxane (PDMS), Dacron, polyethylene glycol (PEG), polytetrafluoroethylene (PTFE), elastin, silicone, polyethylene, methylcellulose, polyoleandlena, nylon, polybutester, polycaprolactone, polyvinyl alcohol (PVA), polycarbonate urethane (PCU), polyester, polylactic acid (PLA), polyglycolic acid (PGA), polyvinyl pyrrolidone (PVP), polyolefin, hydroxy-terminated polycarbonate, polypropylene, polyurethane (PU), poly-N-acetylglucosamine (PNAG), polyetherether ketone (PEEK), and/or polyether block amide (PEBA). In an example, a proximal bowl-shaped mesh or net can be made from both a metal and a polymer.

In an example, a proximal bowl-shaped mesh or net can be made from metal wires, filaments, or tubes. In an example, a proximal bowl-shaped mesh or net can be braided or woven from metal wires, filaments, or tubes. In an example, a proximal bowl-shaped mesh or net can be made from polymer threads, filaments, or tubes. In an example, a proximal bowl-shaped mesh or net can be braided or woven from polymer, threads, filaments, or tubes. In an example, a proximal bowl-shaped mesh or net can be made from a combination or metal and polymer wires, threads, filaments, or tubes. In an example, a proximal bowl-shaped mesh or net can be braided or woven from a combination or metal and polymer wires, threads, filaments, or tubes.

In an example, the curvature (e.g. diameter or circumference) of a proximal bowl-shaped mesh or net can be remotely changed (e.g. increased or decreased) by a device operator. In an example, the curvature (e.g. diameter or circumference) of a proximal bowl-shaped mesh or net can be remotely changed (e.g. increased or decreased) by a device operator by pulling or pushing a longitudinal member (e.g. a wire, filament, coil, or string). In an example, the curvature (e.g. diameter or circumference) of a proximal bowl-shaped mesh or net can be remotely changed (e.g. increased or decreased) by a device operator by rotating a longitudinal member (e.g. a wire, filament, coil, or string). In an example, the curvature (e.g. diameter or circumference) of a proximal bowl-shaped mesh or net can be remotely changed (e.g. increased or decreased) by a device operator by activating an electromagnet. In an example, the curvature (e.g. diameter or circumference) of a proximal bowl-shaped mesh or net can be remotely changed (e.g. increased or decreased) by a device operator by applying electrical energy. In an example, the curvature (e.g. diameter or circumference) of a proximal bowl-shaped mesh or net can be remotely changed (e.g. increased or decreased) by a device operator by activating an electromagnetic field.

In an example, the shape of a proximal bowl-shaped mesh or net can be remotely changed (e.g. increased or decreased) by a device operator. In an example, the shape of a proximal bowl-shaped mesh or net can be remotely changed (e.g. increased or decreased) by a device operator by pulling or pushing a longitudinal member (e.g. a wire, filament, coil, or string). In an example, the shape of a proximal bowl-shaped mesh or net can be remotely changed (e.g. increased or decreased) by a device operator by rotating a longitudinal member (e.g. a wire, filament, coil, or string). In an example, the shape of a proximal bowl-shaped mesh or net can be remotely changed (e.g. increased or decreased) by a device operator by activating an electromagnet. In an example, the shape of a proximal bowl-shaped mesh or net can be remotely changed (e.g. increased or decreased) by a device operator by applying electrical energy. In an example, the shape of a proximal bowl-shaped mesh or net can be remotely changed (e.g. increased or decreased) by a device operator by activating an electromagnetic field.

In an example, the width (e.g. diameter or circumference) of a proximal bowl-shaped mesh or net can be remotely changed (e.g. increased or decreased) by a device operator. In an example, the width (e.g. diameter or circumference) of a proximal bowl-shaped mesh or net can be remotely changed (e.g. increased or decreased) by a device operator by pulling or pushing a longitudinal member (e.g. a wire, filament, coil, or string). In an example, the width (e.g. diameter or circumference) of a proximal bowl-shaped mesh or net can be remotely changed (e.g. increased or decreased) by a device operator by rotating a longitudinal member (e.g. a wire, filament, coil, or string). In an example, the width (e.g. diameter or circumference) of a proximal bowl-shaped mesh or net can be remotely changed (e.g. increased or decreased) by a device operator by activating an electromagnet. In an example, the width (e.g. diameter or circumference) of a proximal bowl-shaped mesh or net can be remotely changed (e.g. increased or decreased) by a device operator by applying electrical energy. In an example, the width (e.g. diameter or circumference) of a proximal bowl-shaped mesh or net can be remotely changed (e.g. increased or decreased) by a device operator by activating an electromagnetic field.

In an example, a proximal bowl-shaped mesh or net can have a compressed first configuration as it is conveyed through a catheter to an aneurysm sac and an expanded second configuration within the aneurysm sac. In an example, a proximal bowl-shaped mesh or net can be folded as it is delivered through a catheter to an aneurysm sac. In an example, a proximal bowl-shaped mesh or net can have cross-sectional folds as it is delivered through a catheter to an aneurysm sac. In an example, a proximal bowl-shaped mesh or net can have longitudinal folds as it is delivered through a catheter to an aneurysm sac. In an example, a proximal bowl-shaped mesh or net can have radial folds as it is delivered through a catheter to an aneurysm sac. In an example, a proximal bowl-shaped mesh or net can have a coiled, scrolled, or spiraled configuration as it is delivered through a catheter to an aneurysm sac.

In an example, a proximal bowl-shaped mesh or net can self-expand after release from a catheter into an aneurysm sac. In an example, a proximal bowl-shaped mesh or net can be made from shape memory material and can self-expand after release from a catheter into an aneurysm sac. In an example, a proximal bowl-shaped mesh or net can be expanded after insertion into an aneurysm sac, by application of electrical current to the mesh or net. In an example, a proximal bowl-shaped mesh or net can be expanded after insertion into an aneurysm sac, by pulling a thread, string, suture, or wire which is attached to the mesh or net. In an example, a proximal bowl-shaped mesh or net can be expanded after insertion into an aneurysm sac, by pulling a thread, string, suture, or wire which is attached to the distal end and/or surface of mesh or net.

In an example, a proximal bowl-shaped mesh or net can be radially-compressed for delivery through a catheter into an aneurysm sac and then radially-expanded after insertion into the aneurysm sac. In an example, a proximal bowl-shaped mesh or net and have a first configuration with a first diameter and a second configuration with a second diameter, wherein the mesh or net is in the first configuration while being delivered through a catheter into an aneurysm sac and is expanded to the second configuration after having been inserted into the aneurysm sac, and wherein the second diameter is between 10% and 50% larger than the first diameter.

In an example, a proximal bowl-shaped mesh or net and have a first configuration with a first diameter and a second configuration with a second diameter, wherein the mesh or net is in the first configuration while being delivered through a catheter into an aneurysm sac and is expanded to the second configuration after having been inserted into the aneurysm sac, and wherein the second diameter is between 40% and 100% larger than the first diameter. In an example, a proximal bowl-shaped mesh or net and have a first configuration with a first diameter and a second configuration with a second diameter, wherein the mesh or net is in the first configuration while being delivered through a catheter into an aneurysm sac and is expanded to the second configuration after having been inserted into the aneurysm sac, and wherein the second diameter is more than twice the first diameter.

In an example, a proximal bowl-shaped mesh or net can be compressed during delivery through a catheter to an aneurysm sac and then expanded within the aneurysm sac. In an example, a proximal bowl-shaped mesh or net can self-expand within an aneurysm sac after being released from a delivery catheter. In an example, a proximal bowl-shaped mesh or net can be made with shape memory material and can self-expand within an aneurysm sac after being released from a delivery catheter.

In an example, there can be an opening, hole, tube, and/or lumen in a proximal bowl-shaped mesh or net through which embolic members can be inserted into an aneurysm sac and/or into a distal globular mesh or net. In an example, there can be a central opening, hole, tube, and/or lumen in a proximal bowl-shaped mesh or net through which embolic members can be inserted into an aneurysm sac and/or into a distal globular mesh or net. In an example, there can be an opening, hole, tube, and/or lumen in the center of a proximal bowl-shaped mesh or net through which embolic members can be inserted into an aneurysm sac and/or into a distal globular mesh or net.

In an example, there can be an opening, hole, tube, and/or lumen in a proximal bowl-shaped mesh or net through which embolic members (e.g. coils, microsponges, hydrogels, or string-of-pearls connected embolic components) can be inserted into an aneurysm sac and/or into a distal globular mesh or net. In an example, there can be an opening, hole, tube, and/or lumen in a proximal bowl-shaped mesh or net through which embolizing (e.g. congealing) liquid (or gel) can be inserted into an aneurysm sac and/or into a distal globular mesh or net.

In an example, there can be a tube or cylinder through a proximal bowl-shaped mesh through which embolic members or material (e.g. coils, microsponges, hydrogels, beads, or embolizing liquid) can be inserted into the aneurysm sac and/or into the distal globular mesh or net. In an example, there can be a tube or cylinder through the center of a proximal bowl-shaped mesh through which embolic members or material (e.g. coils, microsponges, hydrogels, beads, or embolizing liquid) can be inserted into the aneurysm sac and/or into the distal globular mesh or net. In an example, there can be a closure mechanism (e.g. a valve or plug) which enables the operator of the device to close this tube or cylinder after embolic members have been inserted.

In an example, there can be a tube or cylinder through a proximal bowl-shaped mesh or net through which embolizing (e.g. congealing) liquid (or gel) can be inserted into the aneurysm sac and/or into the distal globular mesh or net. In an example, there can be a tube or cylinder through the center of a proximal bowl-shaped mesh or net through which embolizing (e.g. congealing) liquid (or gel) can be inserted into the aneurysm sac and/or into the distal globular mesh or net. In an example, there can be a closure mechanism (e.g. a valve or plug) which enables the operator of the device to close this tube or cylinder after the embolizing (e.g. congealing) liquid (or gel) has been inserted.

In an example, a proximal bowl-shaped mesh or net can have quadrilateral holes or openings. In an example, a proximal bowl-shaped mesh or net can have diamond-shaped holes or openings. In an example, a proximal bowl-shaped mesh or net can have triangular holes or openings. In an example, a proximal bowl-shaped mesh or net can have hexagonal holes or openings. In an example, a proximal bowl-shaped mesh or net can be a honeycomb mesh or net with hexagonal holes or openings. In an example, a proximal bowl-shaped mesh or net can have circular holes or openings.

In an example, a distal globular mesh or net can have a spherical shape. In an example, a distal globular mesh or net can have an ellipsoidal shape. In an example, a distal globular mesh or net can have an oblate spheroid shape. In an example, a distal globular mesh or net can have a first configuration while being delivered through a catheter to an aneurysm sac and a second configuration after being expanded in the aneurysm sac. In an example, the first configuration can comprise an ellipsoidal shape and the second configuration can comprise a spherical shape. In an example, the first configuration can comprise a football shape and the second configuration can comprise a baseball shape.

In an example, a distal globular mesh or net can have a Saturn shape (e.g. with a wider circumferential ring portion). In an example, a distal globular mesh or net can have a pear shape. In an example, a distal globular mesh or net can have an hourglass, figure eight, or dumbbell shape. In an example, a distal globular mesh or net can have an apple or barrel shape. In an example, a distal globular mesh or net can have a pumpkin shape. In an example, a distal globular mesh or net can have a toroidal or doughnut shape.

In an example, the size of a distal globular mesh or net can be remotely adjusted by the operator of a device after the mesh or net has been inserted into an aneurysm sac. In an example, the width of a distal globular mesh or net can be remotely adjusted by the operator of the device after the mesh or net has been inserted into an aneurysm sac. In an example, the height of a distal globular mesh or net can be remotely adjusted by the operator of the device after the mesh or net has been inserted into an aneurysm sac. In an example, the shape of a distal globular mesh or net can be remotely adjusted by the operator of the device after the mesh or net has been inserted into an aneurysm sac.

In an example, the size of a distal globular mesh or net can be remotely adjusted by the application of electrical energy to selected portions of the mesh or net after the mesh or net has been inserted into an aneurysm sac. In an example, the width of a distal globular mesh or net can be remotely adjusted by the application of electrical energy to selected portions of the mesh or net after the mesh or net has been inserted into an aneurysm sac. In an example, the height of a distal globular mesh or net can be remotely adjusted by the application of electrical energy to selected portions of the mesh or net after the mesh or net has been inserted into an aneurysm sac. In an example, the shape of a distal globular mesh or net can be remotely adjusted by the application of electrical energy to selected portions of the mesh or net after the mesh or net has been inserted into an aneurysm sac.

In an example, a distal globular mesh or net can have a shape selected from the group consisting of: ovaloid, spherical, barrel shaped, hourglass shaped, lobed, oblate spheroid, cardioid, apple shaped, bulbous, prolate spheroid, convex, globular, ball shaped, and ellipsoidal. In an example, a distal globular mesh or net can have post-expansion shape which is selected from the group consisting of: ovaloid, spherical, barrel shaped, hourglass shaped, lobed, oblate spheroid, cardioid, apple shaped, bulbous, prolate spheroid, convex, globular, ball shaped, and ellipsoidal. In an example, a distal globular mesh or net can have a cross-sectional shape like that of a whale tail. In an example, a distal globular mesh or net can have a cross-sectional shape which is just a fluke.

In an example, there can be a lateral cross-section of a distal globular mesh or net, wherein this cross-section is perpendicular to the proximal-to-distal axis of the mesh or net, and wherein this cross-section has a circular or elliptical shape. In an example, there can be a lateral cross-section of a distal globular mesh or net, wherein this cross-section is perpendicular to the proximal-to-distal axis of the mesh or net, and wherein this cross-section has an undulating, wavy, or sinusoidal shape.

In an example, there can be a longitudinal cross-section of a distal globular mesh or net, wherein this cross-section is parallel to the proximal-to-distal axis of the mesh or net, and wherein this cross-section has a circular or elliptical shape. In an example, there can be a longitudinal cross-section of a distal globular mesh or net, wherein this cross-section is parallel to the proximal-to-distal axis of the mesh or net, and wherein this cross-section has an hourglass or figure-eight shape. In an example, there can be a longitudinal cross-section of a distal globular mesh or net, wherein this cross-section is parallel to the proximal-to-distal axis of the mesh or net, and wherein this cross-section has a pear shape. In an example, there can be a longitudinal cross-section of a distal globular mesh or net, wherein this cross-section is parallel to the proximal-to-distal axis of the mesh or net, and wherein this cross-section has an undulating, wavy, or sinusoidal shape.

In an example, a distal globular mesh or net can be a mesh. In an example, a distal globular mesh or net can be a braided mesh. In an example, a distal globular mesh or net can be a woven mesh. In an example, a distal globular mesh or net can be a 3D-printed mesh. In an example, a distal globular mesh or net can be a mesh which is made by using a laser to make holes in a balloon (or other elastic convex structure).

In an example, a distal globular mesh or net can have quadrilateral holes or openings. In an example, a distal globular mesh or net can have diamond-shaped holes or openings In an example, a distal globular mesh or net can have triangular holes or openings. In an example, a distal globular mesh or net can have hexagonal holes or openings. In an example, a distal globular mesh or net can be a honeycomb mesh or net with hexagonal holes or openings. In an example, a distal globular mesh or net can have circular holes or openings.

In an example, a distal globular mesh or net can have a resilient circumferential ring or band. In an example, a distal globular mesh or net can have a resilient ring or band around its circumference. In an example, a distal globular mesh or net can have a resilient ring or band around its central circumference. In an example, a distal globular mesh or net can have a resilient ring or band around its central circumference, wherein this ring or band is more resilient (e.g. less flexible or lower durometer) than the rest of the mesh or net. In an example, a distal globular mesh or net can have a shape like the planet Saturn, comprising a generally globular central structure with a protruding ring or band around its central circumference. In an example, a distal globular mesh or net can comprise a generally globular central structure with an outwardly-protruding ring or band around its central circumference.

In an example, a distal globular mesh or net can have circular holes (e.g. holes, openings, or pores). In an example, a distal globular mesh or net can have hexagonal holes (e.g. holes, openings, or pores). In an example, a distal globular mesh or net can have triangular holes (e.g. holes, openings, or pores). In an example, a distal globular mesh or net can have quadrilateral holes (e.g. holes, openings, or pores). In an example, a distal globular mesh or net can have holes (e.g. holes, openings, or pores) which are smaller than embolic members which are inserted into the mesh or net. In an example, a distal globular mesh or net can have holes (e.g. holes, openings, or pores) which are less than half of the size of embolic members which are inserted into the mesh or net.

In an example, a distal globular mesh or net can be made by braiding. In an example, a distal globular mesh or net can be made by braiding wires, tubes, and/or filaments. In an example, a distal globular mesh or net can be made by weaving. In an example, a distal globular mesh or net can be made by weaving wires, tubes, and/or filaments. In an example, a distal globular mesh or net can be made by 3D printing. In an example, a distal globular mesh or net can be made by 3D printing wires, tubes, and/or filaments. In an example, a distal globular mesh or net can comprise a metal mesh or net. In an example, a distal globular mesh or net can comprise a polymer mesh or net. In an example, a distal globular mesh or net can comprise be a stent. In an example, a distal globular mesh or net can be made with shape-memory material. In an example, a distal globular mesh or net can be a wire mesh, lattice, or net. In an example, one or more (circular) elastic bands or rings can be woven or braided into a distal globular mesh or net.

In an example, a distal portion of a distal globular mesh or net can be inverted into itself (or everted over itself). In an example, the distal end of a distal globular mesh or net can be inverted into itself (or everted over itself). In an example, the distal end of a distal globular mesh or net can be the inverted end of a tubular mesh. In an example, the distal end of a distal globular mesh or net can be the radially-constrained and inverted end of a tubular mesh.

In an example, a distal globular mesh or net can be braided or woven from longitudinal wires, tubes, or filaments with longitudinal orientations which are substantially parallel with a proximal-to-distal axis of the mesh or net. In an example, a distal globular mesh or net can be braided or woven from longitudinal wires, tubes, or filaments with longitudinal orientations which are substantially perpendicular to a proximal-to-distal axis of the mesh or net. In an example, a distal globular mesh or net can be braided or woven from longitudinal wires, tubes, or filaments with longitudinal orientations which are substantially parallel with a circumference of the mesh or net. In an example, a distal globular mesh or net can be braided or woven from longitudinal wires, tubes, or filaments with longitudinal orientations which are substantially concentric with a circumference of the mesh or net.

In an example, a distal globular mesh or net can be braided or woven from helical longitudinal wires, tubes, or filaments. In an example, a distal globular mesh or net can be braided or woven from: a first set of wires, tubes, or filaments which are substantially parallel with a proximal-to-distal axis of the mesh or net; and a second set of wires, tubes, or filaments which are substantially perpendicular to a proximal-to-distal axis of the mesh or net. In an example, a distal globular mesh or net can be braided or woven from: a first set of wires, tubes, or filaments which are substantially parallel with a proximal-to-distal axis of the mesh or net; and a second set of wires, tubes, or filaments which are substantially concentric with a circumference of the mesh or net.

In an example, a distal globular mesh or net can be braided or woven from wires, tubes, or filaments which extend radially outward from the poles of the globular mesh or net. In an example, a distal globular mesh or net can be braided or woven from: a first set of wires, tubes, or filaments which extend radially outward from the poles of the globular mesh or net; and a second set of wires, tubes, or filaments which are substantially concentric with a circumference of the mesh or net. In an example, a distal globular mesh or net can be braided or woven from: a first set of wires, tubes, or filaments which extend radially outward from the poles of the globular mesh or net; and a second set of wires, tubes, or filaments which are helical.

In an example, a distal globular mesh or net can be braided or woven from undulating, wavy, and/or sinusoidal wires, tubes, or filaments. In an example, a distal globular mesh or net can be braided or woven from: a first set of wires, tubes, or filaments which are undulating, wavy, and/or sinusoidal; and a second set of wires, tubes, or filaments which are not undulating, wavy, and/or sinusoidal. In an example, a distal globular mesh or net can comprise a braided or woven combination of undulating and helical wires, tubes, or filaments.

In an example, a distal globular mesh or net can have one layer. In an example, a distal globular mesh or net can have two or more layers. In an example, a distal globular mesh or net with two layers can be made by radially-constraining and inverting a tubular mesh at two locations. In an example, a distal globular mesh or net with two layers can be made by radially-constraining a tubular mesh at two locations and then inverting or everting it. In an example, a distal globular mesh or net with two layers can be made by radially-constraining and inverting a tubular mesh at three locations.

In an example, a distal globular mesh or net can have an inner polymer mesh layer and an outer metal mesh layer. In an example, a distal globular mesh or net can have an inner layer, an outer layer, and a middle layer between the inner and outer layers. In an example, the inner and outer layers can be metal meshes or nets and the middle layer can be a polymer mesh or net. In an example, a distal globular mesh or net can comprise a metal mesh or net which has been coated with a polymer material. In an example, a distal globular mesh or net can comprise a metal mesh or net into which has been woven a polymer material. In an example, a distal globular mesh or net can comprise a metal mesh or net which has been coated with a hydrogel material.

In an example, an outer globular mesh or net can have two layers, an inner layer and an outer layer. In an example, the distance between an inner layer or surface of an outer globular mesh or net and an outer layer or surface of the mesh or net can be non-uniform. In an example, the distance between an inner layer or surface of an outer globular mesh or net and the outer layer or surface of the mesh or net can be greater in a longitudinally-central portion of the mesh or net than in proximal or distal portions of the mesh or net. In an example, the distance between an inner layer or surface of an outer globular mesh or net and the outer layer or surface of the mesh or net can be less in a longitudinally-central portion of the mesh or net than in proximal or distal portions of the mesh or net.

In an example, the distal end of a distal globular mesh or net can be inverted. In an example, there can be a distal inversion on the distal end of a globular mesh or net. In an example, the distal end of a distal globular mesh or net can be inverted so as to extend into the interior of the distal globular mesh or net. In an example, a distal inversion can extend inward between 10% and 30% of the interior diameter of a distal globular mesh or net. In an example, a distal inversion can extend inward between 20% and 55% of the interior diameter of a distal globular mesh or net.

In an example, a distal inversion can extend inward between 50% and 80% of the interior diameter of a distal globular mesh or net. In an example, a distal inverted end of a distal globular mesh or net can extend in a proximal direction into the interior of the mesh or net and a proximal inverted end of the mesh or net can extend in a distal direction into the interior of the mesh or net. In an example, a distal inverted end of a distal globular mesh or net can extend in a proximal direction into the interior of the mesh or net and a proximal inverted end of the mesh or net can extend in a proximal direction outside the mesh or net.

In an example, a distal globular mesh or net can be made using 3D printing. In an example, a distal globular mesh or net can be made by 3D printing with a polymer material. In an example, a distal globular mesh or net can be made by 3D printing with a silicone-based polymer. In an example, a distal globular mesh or net can be made by 3D printing with an elastomeric polymer. In an example, a distal globular mesh or net can be made by 3D printing with polydimethylsiloxane (PDMS). In an example, a distal globular mesh or net can be made by 3D printing with liquid metal. In an example, a distal globular mesh or net can be made by using a laser to make holes in a metal film and/or sheet. In an example, a distal globular mesh or net can be made by using a laser to make holes in a polymer film and/or sheet.

In an example, a distal globular mesh or net can be made from a tubular mesh or net. In an example, a distal globular mesh or net can be made by inverting or everting a tubular mesh or net. In an example, a distal globular mesh or net can be made by inverting or everting the ends of a tubular mesh or net. In an example, a distal globular mesh or net can be made by radially-constraining and then inverting (or everting) a tubular mesh or net. In an example, a distal globular mesh or net can be made by radially-constraining and then inverting (or everting) a tubular mesh or net at two locations along the longitudinal axis of the tubular mesh or net. In an example, a distal globular mesh or net can be made by radially-constraining a tubular mesh or net with a ring (e.g. ring, band, cylinder, other annular member, string, or wire) and then inverting (or everting) a tubular mesh or net.

In an example, a distal globular mesh or net can be made by radially-constraining a tubular mesh or net with a ring (e.g. ring, band, cylinder, other annular member, string, or wire) at two locations and then inverting (or everting) a tubular mesh or net at these locations. In an example, a tubular mesh or net can be transformed into a distal globular mesh or net by two rings (e.g. rings, bands, cylinders, other annular members, strings, or wires) which radially-constrain the ends of the tubular mesh. In an example, a tubular mesh can be transformed into a distal globular mesh or net by two rings (e.g. rings, bands, cylinders, other annular members, strings, or wires) which radially-constrain the proximal and distal ends of the tubular mesh. In an example, the distal end of a tubular mesh can be radially-constrained by a distal ring (e.g. ring, band, cylinder, other annular member, string, or wire) and the proximal end of a tubular mesh can be radially-constrained by a proximal ring (e.g. ring, band, cylinder, other annular member, string, or wire) to form a distal globular mesh or net.

In an example, a distal globular mesh or net can be formed by radially-constraining a tubular mesh or net at two different points along the longitudinal axis of the tubular mesh or net. In an example, a distal globular mesh or net can be formed by radially-constraining the ends of a tubular mesh or net. In an example, a distal globular mesh or net can be formed by radially-constraining and inverting a tubular mesh or net at two different points along the longitudinal axis of the tubular mesh or net. In an example, a distal globular mesh or net can be formed by radially-constraining and inverting the ends of a tubular mesh or net. In an example, a distal globular mesh or net can be formed by inverting a tubular mesh or net at two different points along the longitudinal axis of the tubular mesh or net. In an example, a distal globular mesh or net can be formed by inverting the ends of a tubular mesh or net.

In an example, a distal globular mesh or net can be made by 3D printing. In an example, a distal globular mesh or net can be made by 3D printing a polymer into a porous globular structure. In an example, a distal globular mesh or net can be made by creating holes in a globular membrane. In an example, a distal globular mesh or net can be made by using a laser to create holes in a globular membrane. In an example, a distal globular mesh or net can be made by using a laser to create holes in a globular polymer structure.

In an example, a distal globular mesh or net can have uniform density, flexibility, porosity, thickness, and durometer level. In an example, a central portion of a distal globular mesh or net can have a different density, flexibility, porosity, thickness, and/or durometer level than its peripheral portions. In an example, a central portion of a distal globular mesh or net can have a greater density, flexibility, porosity, thickness, and/or durometer level than its peripheral distal portions. In an example, a central portion of a distal globular mesh or net can have a lower density, flexibility, porosity, thickness, and/or durometer level than its peripheral portions.

In an example, a distal portion of a distal globular mesh or net and a proximal portion of this distal globular mesh or net can have the same levels of porosity, thickness, elasticity, flexibility, durometer, density, compliance, conformability, and/or malleability. In an example, a distal portion of a distal globular mesh or net and a proximal portion of this distal globular mesh or net can have a different levels of porosity, thickness, elasticity, flexibility, durometer, density, compliance, conformability, and/or malleability. In an example, a distal portion of a distal globular mesh or net can have a first level of porosity, thickness, elasticity, flexibility, durometer, density, compliance, conformability, and/or malleability; and a proximal portion of this distal globular mesh or net can have a second level of porosity, thickness, elasticity, flexibility, durometer, density, compliance, conformability, and/or malleability; wherein the first level is greater than the second level.

In an example, a distal portion of a distal globular mesh or net can be more compliant than a proximal portion of this distal globular mesh or net. In an example, a distal portion of a distal globular mesh or net can be less elastic than a proximal portion of this distal globular mesh or net. In an example, a distal portion of a distal globular mesh or net can be more conformable than a proximal portion of this distal globular mesh or net. In an example, a distal portion of a distal globular mesh or net can be more elastic than a proximal portion of this distal globular mesh or net. In an example, a distal portion of a distal globular mesh or net can be more flexible than a proximal portion of this distal globular mesh or net. In an example, a distal portion of a distal globular mesh or net can be made from a metal and a proximal portion of this distal globular mesh or net can be made from a polymer.

In an example, a distal portion of a distal globular mesh or net can be more malleable than a proximal portion of this distal globular mesh or net. In an example, a distal portion of a distal globular mesh or net can be more porous than a proximal portion of this distal globular mesh or net. In an example, a distal portion of a distal globular mesh or net can be thicker than a proximal portion of this distal globular mesh or net. In an example, a distal portion of a distal globular mesh or net can have a larger hole size than that of a proximal portion of this distal globular mesh or net. In an example, a distal portion of a distal globular mesh or net can have a higher durometer level than a proximal portion of this distal globular mesh or net. In an example, a distal portion of a distal globular mesh or net can be made from a polymer and a proximal portion of this distal globular mesh or net can be made from a metal.

In an example, a distal portion of a distal globular mesh or net can be made with a greater percentage of polymer material than a proximal portion of this distal globular mesh or net. In an example, a distal portion of a distal globular mesh or net can be less compliant than a proximal portion of this distal globular mesh or net. In an example, a distal portion of a distal globular mesh or net can have a lower durometer level than a proximal portion of this distal globular mesh or net. In an example, a distal portion of a distal globular mesh or net can be less flexible than a proximal portion of this distal globular mesh or net. In an example, a distal portion of a distal globular mesh or net can be made with a lower percentage of polymer material than a proximal portion of this distal globular mesh or net.

In an example, a distal portion of a distal globular mesh or net can be less malleable than a proximal portion of this distal globular mesh or net. In an example, a distal portion of a distal globular mesh or net can be made from a different material than a proximal portion of this distal globular mesh or net. In an example, a distal portion of a distal globular mesh or net can have a first level of porosity, thickness, elasticity, flexibility, durometer, density, compliance, conformability, and/or malleability; and a proximal portion of this distal globular mesh or net can have a second level of porosity, thickness, elasticity, flexibility, durometer, density, compliance, conformability, and/or malleability; wherein the first level is less than the second level.

In an example, a distal portion of a distal globular mesh or net can be less conformable than a proximal portion of this distal globular mesh or net. In an example, a distal portion of a distal globular mesh or net can have a smaller hole size than that of distal globular mesh or net. In an example, a distal portion of a distal globular mesh or net can have a different hole shape than a proximal portion of this distal globular mesh or net. In an example, a distal portion of a distal globular mesh or net can be less porous than a proximal portion of this distal globular mesh or net. In an example, a distal portion of a distal globular mesh or net can be thinner than a proximal portion of this distal globular mesh or net.

In an example, a proximal portion of a distal globular mesh or net can have a different density, flexibility, porosity, thickness, and/or durometer level than its distal portion. In an example, a proximal portion of a distal globular mesh or net can have a greater density, flexibility, porosity, thickness, and/or durometer level than its distal portion. In an example, a proximal portion of a distal globular mesh or net can have a lower density, flexibility, porosity, thickness, and/or durometer level than its distal portion. In an example, a proximal half of a distal globular mesh or net can have a different density, flexibility, porosity, thickness, and/or durometer level than its distal half. In an example, a proximal half of a distal globular mesh or net can have a greater density, flexibility, porosity, thickness, and/or durometer level than its distal half. In an example, a proximal half of a distal globular mesh or net can have a lower density, flexibility, porosity, thickness, and/or durometer level than its distal half.

In an example, the distal half of a distal globular mesh or net can be less porous than the proximal half of the mesh or net. In an example, the distal half of a distal globular mesh or net can be more dense than the proximal half of the mesh or net. In an example, the distal half of a distal globular mesh or net can be stiffer than the proximal half of the mesh or net. In an example, the distal half of a distal globular mesh or net can less elastic than the proximal half of the mesh or net. In an example, the distal half of a distal globular mesh or net can have a higher durometer level than the proximal half of the mesh or net. In an example, the distal half of a distal globular mesh or net can be wider than the proximal half of the mesh or net.

In an example, the proximal half of a distal globular mesh or net can be less porous than the distal half of the mesh or net. In an example, the proximal half of a distal globular mesh or net can be more dense than the distal half of the mesh or net. In an example, the proximal half of a distal globular mesh or net can be stiffer than the distal half of the mesh or net. In an example, the proximal half of a distal globular mesh or net can less elastic than the distal half of the mesh or net. In an example, the proximal half of a distal globular mesh or net can have a higher durometer level than the distal half of the mesh or net. In an example, the proximal half of a distal globular mesh or net can be wider than the distal half of the mesh or net.

In an example, the distal third of a distal globular mesh or net can be less porous than the proximal two-thirds of the mesh or net. In an example, the distal third of a distal globular mesh or net can be more dense than the proximal two-thirds of the mesh or net. In an example, the distal third of a distal globular mesh or net can be stiffer than the proximal two-thirds of the mesh or net. In an example, the distal third of a distal globular mesh or net can less elastic than the proximal two-thirds of the mesh or net. In an example, the distal third of a distal globular mesh or net can have a higher durometer level than the proximal two-thirds of the mesh or net. In an example, the distal third of a distal globular mesh or net can be wider than the proximal two-thirds of the mesh or net.

In an example, the proximal third of a distal globular mesh or net can be less porous than the distal two-thirds of the mesh or net. In an example, the proximal third of a distal globular mesh or net can be more dense than the distal two-thirds of the mesh or net. In an example, the proximal third of a distal globular mesh or net can be stiffer than the distal two-thirds of the mesh or net. In an example, the proximal third of a distal globular mesh or net can less elastic than the distal two-thirds of the mesh or net. In an example, the proximal third of a distal globular mesh or net can have a higher durometer level than the distal two-thirds of the mesh or net. In an example, the proximal third of a distal globular mesh or net can be wider than the distal two-thirds of the mesh or net.

In an example, a proximal portion of a distal globular mesh or net can be more dense than a distal portion of a distal globular mesh or net. In an example, a proximal portion of a distal globular mesh or net can have a higher durometer level than a distal portion of a distal globular mesh or net. In an example, a proximal portion of a distal globular mesh or net can be less porous than a distal portion of a distal globular mesh or net. In an example, a proximal portion of a distal globular mesh or net can be less elastic than a distal portion of a distal globular mesh or net. In an example, wires, tubes, and/or filaments in a proximal portion of a distal globular mesh or net can thicker than those in a distal portion of the distal globular mesh or net.

In an example, a proximal portion of a distal globular mesh or net can be at least 50% more dense than a distal portion of a distal globular mesh or net. In an example, a proximal portion of a distal globular mesh or net can have a durometer level which is at least 50% more than that of a distal portion of the distal globular mesh or net. In an example, a proximal portion of a distal globular mesh or net can be less than half as porous as a distal portion of a distal globular mesh or net. In an example, wires, tubes, and/or filaments in a proximal portion of a distal globular mesh or net can be at least 50% thicker than those in a distal portion of the distal globular mesh or net.

In an example, a distal globular mesh or net can be made from a metal. In an example, a distal globular mesh or net can be made from cobalt chromium alloy, nickel-titanium alloy, Nitinol, platinum, tantalum, and/or stainless steel. In an example, a distal globular mesh or net can be made from a polymer. In an example, a distal globular mesh or net can be made from polyethylene terephthalate (PET), polydimethylsiloxane (PDMS), Dacron, polyethylene glycol (PEG), polytetrafluoroethylene (PTFE), elastin, silicone, polyethylene, methylcellulose, polydarton, nylon, polybutester, polycaprolactone, polyvinyl alcohol (PVA), polycarbonate urethane (PCU), polyester, polylactic acid (PLA), polyglycolic acid (PGA), polyvinyl pyrrolidone (PVP), polyolefin, hydroxy-terminated polycarbonate, polypropylene, polyurethane (PU), poly-N-acetylglucosamine (PNAG), polyetherether ketone (PEEK), and/or polyether block amide (PEBA). In an example, a distal globular mesh or net can be made from both a metal and a polymer.

In an example, a distal globular mesh or net can be made from metal wires, filaments, or tubes. In an example, a distal globular mesh or net can be braided or woven from metal wires, filaments, or tubes. In an example, a distal globular mesh or net can be made from polymer threads, filaments, or tubes. In an example, a distal globular mesh or net can be braided or woven from polymer, threads, filaments, or tubes. In an example, a distal globular mesh or net can be made from a combination or metal and polymer wires, threads, filaments, or tubes. In an example, a distal globular mesh or net can be braided or woven from a combination or metal and polymer wires, threads, filaments, or tubes.

In an example, the curvature (e.g. diameter or circumference) of a distal globular mesh or net can be remotely changed (e.g. increased or decreased) by a device operator. In an example, the curvature (e.g. diameter or circumference) of a distal globular mesh or net can be remotely changed (e.g. increased or decreased) by a device operator by pulling or pushing a longitudinal member (e.g. a wire, filament, coil, or string). In an example, the curvature (e.g. diameter or circumference) of a distal globular mesh or net can be remotely changed (e.g. increased or decreased) by a device operator by rotating a longitudinal member (e.g. a wire, filament, coil, or string). In an example, the curvature (e.g. diameter or circumference) of a distal globular mesh or net can be remotely changed (e.g. increased or decreased) by a device operator by activating an electromagnet. In an example, the curvature (e.g. diameter or circumference) of a distal globular mesh or net can be remotely changed (e.g. increased or decreased) by a device operator by applying electrical energy. In an example, the curvature (e.g. diameter or circumference) of a distal globular mesh or net can be remotely changed (e.g. increased or decreased) by a device operator by activating an electromagnetic field.

In an example, the shape of a distal globular mesh or net can be remotely changed (e.g. increased or decreased) by a device operator. In an example, the shape of a distal globular mesh or net can be remotely changed (e.g. increased or decreased) by a device operator by pulling or pushing a longitudinal member (e.g. a wire, filament, coil, or string). In an example, the shape of a distal globular mesh or net can be remotely changed (e.g. increased or decreased) by a device operator by rotating a longitudinal member (e.g. a wire, filament, coil, or string). In an example, the shape of a distal globular mesh or net can be remotely changed (e.g. increased or decreased) by a device operator by activating an electromagnet. In an example, the shape of a distal globular mesh or net can be remotely changed (e.g. increased or decreased) by a device operator by applying electrical energy. In an example, the shape of a distal globular mesh or net can be remotely changed (e.g. increased or decreased) by a device operator by activating an electromagnetic field.

In an example, the width (e.g. diameter or circumference) of a distal globular mesh or net can be remotely changed (e.g. increased or decreased) by a device operator. In an example, the width (e.g. diameter or circumference) of a distal globular mesh or net can be remotely changed (e.g. increased or decreased) by a device operator by pulling or pushing a longitudinal member (e.g. a wire, filament, coil, or string). In an example, the width (e.g. diameter or circumference) of a distal globular mesh or net can be remotely changed (e.g. increased or decreased) by a device operator by rotating a longitudinal member (e.g. a wire, filament, coil, or string). In an example, the width (e.g. diameter or circumference) of a distal globular mesh or net can be remotely changed (e.g. increased or decreased) by a device operator by activating an electromagnet. In an example, the width (e.g. diameter or circumference) of a distal globular mesh or net can be remotely changed (e.g. increased or decreased) by a device operator by applying electrical energy. In an example, the width (e.g. diameter or circumference) of a distal globular mesh or net can be remotely changed (e.g. increased or decreased) by a device operator by activating an electromagnetic field.

In an example, a distal globular mesh or net can have a compressed first configuration as it is conveyed through a catheter to an aneurysm sac and an expanded second configuration within the aneurysm sac. In an example, a distal globular mesh or net can be folded as it is delivered through a catheter to an aneurysm sac. In an example, a distal globular mesh or net can have cross-sectional folds as it is delivered through a catheter to an aneurysm sac. In an example, a distal globular mesh or net can have longitudinal folds as it is delivered through a catheter to an aneurysm sac. In an example, a distal globular mesh or net can have radial folds as it is delivered through a catheter to an aneurysm sac. In an example, a distal globular mesh or net can have a coiled, scrolled, or spiraled configuration as it is delivered through a catheter to an aneurysm sac.

In an example, a distal globular mesh or net can self-expand after release from a catheter into an aneurysm sac. In an example, a distal globular mesh or net can be made from shape memory material and can self-expand after release from a catheter into an aneurysm sac. In an example, a distal globular mesh or net can be expanded after insertion into an aneurysm sac, by application of electrical current to the mesh or net. In an example, a distal globular mesh or net can be expanded after insertion into an aneurysm sac, by pulling a thread, string, suture, or wire which is attached to the mesh or net. In an example, a distal globular mesh or net can be expanded after insertion into an aneurysm sac, by pulling a thread, string, suture, or wire which is attached to the distal end and/or surface of mesh or net.

In an example, a distal globular mesh or net can be compressed during delivery through a catheter to an aneurysm sac and then expanded within the aneurysm sac. In an example, a distal globular mesh or net can self-expand within an aneurysm sac after being released from a delivery catheter. In an example, a distal globular mesh or net can be made with shape memory material and can self-expand within an aneurysm sac after being released from a delivery catheter. In an example, a distal globular mesh or net can be expanded within an aneurysm sac by being filled with embolic members and/or embolic material. In an example, a distal globular mesh or net can be expanded within an aneurysm sac by insertion of embolic members and/or embolic material into the distal globular mesh or net.

In an example, a distal globular mesh or net can self-expand to a first extent within an aneurysm sac and then be further expanded to a second extent by being filled with embolic members and/or embolic material. In an example, a distal globular mesh or net can self-expand to a first extent within an aneurysm sac and then be further expanded, to a second extent, due to pressure from the accumulation of embolic members and/or embolic material within its interior and/or distal-facing concavity. In an example, a distal globular mesh or net can self-expand to a first extent within an aneurysm sac and then be further expanded to a second (larger) size by being filled with embolic members and/or embolic material. In an example, a distal globular mesh or net can self-expand to a generally regular and/or axially-symmetric spherical or ellipsoidal shape and then be further expanded to a somewhat irregular and/or axially-asymmetric shape by being filled with embolic members and/or embolic material.

In an example, a distal globular mesh or net can be expanded by being filled with embolic members (e.g. coils, hydrogels, microsponges, beads, or string-of-pearls threaded embolic components). In an example, a distal globular mesh or net can be expanded by being filled with embolizing liquid or gel (e.g. congealing liquid or gel). In an example, a distal globular mesh or net can be expanded by being filled with embolic material or members (e.g. coils, hydrogels, microsponges, beads, string-of-pearls threaded embolic components, or embolic liquid or gel) which are inserted into the mesh or net through a central proximal opening in the mesh or net.

In an example, a distal globular mesh or net can be radially-compressed for delivery through a catheter into an aneurysm sac and then radially-expanded after insertion into the aneurysm sac. In an example, a distal globular mesh or net and have a first configuration with a first diameter and a second configuration with a second diameter, wherein the mesh or net is in the first configuration while being delivered through a catheter into an aneurysm sac and is expanded to the second configuration after having been inserted into the aneurysm sac, and wherein the second diameter is between 10% and 50% larger than the first diameter.

In an example, a distal globular mesh or net and have a first configuration with a first diameter and a second configuration with a second diameter, wherein the mesh or net is in the first configuration while being delivered through a catheter into an aneurysm sac and is expanded to the second configuration after having been inserted into the aneurysm sac, and wherein the second diameter is between 40% and 100% larger than the first diameter. In an example, a distal globular mesh or net and have a first configuration with a first diameter and a second configuration with a second diameter, wherein the mesh or net is in the first configuration while being delivered through a catheter into an aneurysm sac and is expanded to the second configuration after having been inserted into the aneurysm sac, and wherein the second diameter is more than twice the first diameter.

In an example, there can be an opening, hole, tube, and/or lumen in a distal globular mesh or net through which embolic members can be inserted into the distal globular mesh or net. In an example, there can be a central opening, hole, tube, and/or lumen in a distal globular mesh or net through which embolic members can be inserted into the distal globular mesh or net. In an example, there can be an opening, hole, tube, and/or lumen in the center of a distal globular mesh or net through which embolic members can be inserted into the distal globular mesh or net.

In an example, there can be an opening, hole, tube, and/or lumen in a distal globular mesh or net through which embolic members (e.g. coils, microsponges, hydrogels, or string-of-pearls connected embolic components) can be inserted into the distal globular mesh or net. In an example, there can be an opening, hole, tube, and/or lumen in a distal globular mesh or net through which embolizing (e.g. congealing) liquid (or gel) can be inserted into the distal globular mesh or net.

In an example, there can be a tube or cylinder through a distal globular mesh or net through which embolic members or material (e.g. coils, microsponges, hydrogels, beads, or embolizing liquid) can be inserted into the aneurysm sac and/or into the distal globular mesh or net. In an example, there can be a tube or cylinder through the center of a distal globular mesh or net through which embolic members or material (e.g. coils, microsponges, hydrogels, beads, or embolizing liquid) can be inserted into the aneurysm sac and/or into the distal globular mesh or net. In an example, there can be a closure mechanism (e.g. a valve or plug) which enables the operator of the device to close this tube or cylinder after embolic members or material has been inserted.

In an example, an intrasacular aneurysm occlusion device can comprise: a proximal bowl-shaped mesh or net which is inserted into an aneurysm sac and expanded to span a neck of the aneurysm sac; and a distal globular mesh or net which is inserted into the aneurysm sac between the proximal bowl-shaped mesh or net and a distal dome of the aneurysm sac to hold the proximal bowl-shaped mesh or net against the neck of the aneurysm. In an example, an intrasacular aneurysm occlusion device can comprise: a proximal bowl-shaped mesh or net which is inserted into an aneurysm sac and expanded to span a neck of the aneurysm sac; a distal globular mesh or net which is inserted into the aneurysm sac between the proximal bowl-shaped mesh or net and a distal dome of the aneurysm sac to hold the proximal bowl-shaped mesh or net against the neck of the aneurysm; and a connector which connects (e.g. connects or attaches) the proximal bowl-shaped mesh or net and the distal globular mesh or net to each other.

In an example, a distal globular mesh or net can be nested within a proximal bowl-shaped mesh or net. In an example, a proximal portion of a distal globular mesh or net can be nested within a proximal bowl-shaped mesh or net. In an example, the proximal half of a distal globular mesh or net can be nested within a proximal bowl-shaped mesh or net. In an example, between 20% and 40% of a distal globular mesh or net can be nested within the concavity of a proximal bowl-shaped mesh or net. In an example, between 30% and 66% of a distal globular mesh or net can be nested within the concavity of a proximal bowl-shaped mesh or net.

In an example, a proximal bowl-shaped mesh or net and a distal globular mesh or net can have a first configuration in which they are not nested as they travel through a catheter to an aneurysm sac and a second configuration in which they are nested after they have been deployed in the aneurysm sac. In an example, a proximal bowl-shaped mesh or net and a distal globular mesh or net can have a first configuration in which they do not overlap as they travel through a catheter to an aneurysm sac and a second configuration in which they do overlap after they have been deployed in the aneurysm sac.

In an example, distal and proximal stents may not overlap in their first configurations as they travel through a catheter, but they do overlap after they are deployed in their second configurations within an aneurysm sac. In an example, distal and proximal stents can have central longitudinal axes which do not overlap in their first configurations within a catheter, but which do overlap in their second configurations within an aneurysm sac. In an example, a proximal bowl-shaped mesh or net can cover an aneurysm neck and a distal globular mesh or net can fit into a distal convex surface of the proximal bowl-shaped mesh or net.

In an example, a proximal bowl-shaped mesh or net and a distal globular mesh or net can be coaxial. In an example, a proximal bowl-shaped mesh or net and a distal globular mesh or net can (at least partially) share a common longitudinal (e.g. proximal to distal) axis. In an example, the longitudinal axes of a proximal bowl-shaped mesh or net and a distal globular mesh or net can be aligned. In an example, a proximal bowl-shaped mesh or net and a distal globular mesh or net can have a first configuration in which they are not coaxial as they travel through a catheter toward an aneurysm sac and can have a second configuration in which they are coaxial after they have been deployed in the aneurysm sac.

In an example, a proximal bowl-shaped mesh or net and a distal globular mesh or net can be made by radially-constraining (e.g. with a band, ring, or cylinder), inverting (e.g. folding and overlapping inward), and/or everting (e.g. folding and overlapping outward) a single continuous structure. In an example, a proximal bowl-shaped mesh or net and a distal globular mesh or net can be made by radially-constraining (e.g. with a band, ring, or cylinder), inverting (e.g. folding and overlapping inward), and/or everting (e.g. folding and overlapping outward) a tubular mesh or net. In an example, a proximal bowl-shaped mesh or net and a distal globular mesh or net can be made from a sequence of two globular meshes or nets, wherein the proximal one is compressed to form the proximal bowl-shaped mesh or net and the distal globular mesh or net fits into concavity of the proximal bowl-shaped mesh or net. In an example, a tubular mesh can be transformed into a “ball in a bowl” mesh or net device by: radially constraining the distal end of the tubular mesh (e.g. with a band, ring, or cylinder); radially constraining (e.g. with a band, ring, or cylinder) a middle section of the tubular mesh; and then everting the proximal portion of the tubular mesh.

In an example, a proximal bowl-shaped mesh and a distal globular mesh can be made by: radially-constraining the distal end of a tubular mesh, everting a proximal portion of the tubular mesh in a distal direction, and inverting a distal portion of the tubular mesh in a proximal direction. In an example, an intrasacular aneurysm occlusion device with a proximal bowl-shaped mesh and a distal cardioid (e.g. heart-shaped) mesh can be formed by: radially-constraining the proximal end of a tubular mesh, inverting the proximal third of the tubular mesh, and everting the distal third of the tubular mesh. Alternatively, an intrasacular aneurysm occlusion device with a proximal bowl-shaped mesh and a distal globular (e.g. spherical and/or cardioid) mesh can be made by: radially-constraining the distal end of a tubular mesh, inverting the proximal third of the tubular mesh, and everting the distal third of the tubular mesh.

In an example, resilient wider-than-neck (e.g. proximal bowl-shaped) and flexible sac-filling (e.g. distal globular) portions of this device can be different portions of the same structure. In an example, resilient wider-than-neck (e.g. proximal bowl-shaped) and flexible sac-filling (e.g. distal globular) portions of this device can be proximal and distal portions, respectively, of the same structure. In an example, resilient wider-than-neck (e.g. proximal bowl-shaped) and flexible sac-filling (e.g. distal globular) portions of this device can be proximal and distal portions, respectively, of a single continuous structure. In an example, resilient wider-than-neck (e.g. proximal bowl-shaped) and flexible sac-filling (e.g. distal globular) portions of this device can be inner and outer portions or layers, respectively, of the same structure. In an example, resilient wider-than-neck (e.g. proximal bowl-shaped) and flexible sac-filling (e.g. distal globular) portions of this device can be inner and outer portions or layers, respectively, of the same continuous structure. In an example, resilient wider-than-neck (e.g. proximal bowl-shaped) and flexible sac-filling (e.g. distal globular) portions of this device can be different portions of the same continuous embolic structure which is inserted into an aneurysm sac.

In an example, an intrasacular aneurysm occlusion device with a proximal bowl-shaped mesh and a distal cardioid mesh can be made by: radially-constraining the proximal end of a tubular mesh, inverting a portion of the tubular mesh, and everting a portion of the tubular mesh. In an example, an intrasacular aneurysm occlusion device with a proximal bowl-shaped mesh and a distal globular mesh can be made by: radially-constraining the distal end of a tubular mesh, inverting a portion of the tubular mesh, and everting a portion of the tubular mesh. In another example, an intrasacular aneurysm occlusion device with a proximal bowl-shaped mesh and a distal cardioid (e.g. heart-shaped) mesh can be made in the following steps: radially-constraining the distal end of a tubular mesh, inverting the proximal third of the tubular mesh in a distal direction, and everting the distal third of the tubular mesh in a proximal direction.

In an example, a proximal bowl-shaped mesh or net and a distal globular mesh or net can be created from a single continuous mesh tube by radially-constraining the distal end of the tube and everting the tube (in distal direction) over a eversion point somewhere along the proximal half of the length of the tube. The portion of the tube which is distal to the eversion point becomes the globular mesh or net and the portion of the tube which was proximal to the eversion point becomes the proximal bowl-shaped mesh or net.

In an example, an intrasacular aneurysm occlusion device with a proximal bowl-shaped mesh and a distal cardioid-shaped mesh can be made in the following steps: radially-constraining the proximal end of a tubular mesh (e.g. with a ring, band, wire, or string), everting a proximal portion of the tubular mesh in a distal direction, and inverting a distal portion of the tubular mesh in a proximal direction. In one embodiment, an intrasacular aneurysm occlusion device with a proximal bowl-shaped mesh or net and a distal globular (e.g. spherical and/or cardioid) mesh or net can be made by: radially-constraining the proximal end of a tubular mesh, inverting the proximal third of the tubular mesh, and everting the distal third of the tubular mesh. In another example, an intrasacular aneurysm occlusion device with a proximal bowl-shaped mesh and a distal cardioid mesh can be formed by: radially-constraining the distal end of a tubular mesh, inverting the proximal half of the tubular mesh in a distal direction, and everting the distal half of the tubular mesh over the rest of the tubular mesh in a proximal direction.

In an example, a resilient wider-than-neck (e.g. proximal bowl-shaped) portion of this device can comprise the proximal surface of an intrasacular occlusion device and a flexible sac-filling (e.g. distal globular) portion can comprise the distal and lateral surfaces of this intrasacular occlusion device. In an example, a resilient wider-than-neck (e.g. proximal bowl-shaped) portion can be a proximal part, portion, segment, or undulation of this structure and a flexible sac-filling (e.g. distal globular) portion can be a distal (and/or peripheral) part, portion, segment, or undulation of this structure. In an example, a resilient wider-than-neck (e.g. proximal bowl-shaped) portion can be a proximal part, portion, segment, or undulation of an intrasacular aneurysm occlusion device and a flexible sac-filling (e.g. distal globular) portion can be a distal (and/or peripheral) part, portion, segment, or undulation of this device.

In an example, an intrasacular aneurysm occlusion device with a proximal bowl-shaped mesh or net and a distal globular mesh or net can be formed by: radially-constraining the proximal end of a tubular mesh, everting a proximal portion of the tubular mesh, and inverting a distal portion of the tubular mesh. Alternatively, an intrasacular aneurysm occlusion device with a proximal bowl-shaped mesh and a distal cardioid mesh can be made by: radially-constraining the distal end of a tubular mesh, everting a portion of the tubular mesh, and inverting a portion of the tubular mesh. In an example, an intrasacular aneurysm occlusion device with a proximal bowl-shaped mesh and a distal cardioid (e.g. heart-shaped) mesh can be made by: radially-constraining the proximal end of a tubular mesh (e.g. with a ring, band, wire, or string), everting a proximal portion of the tubular mesh, and inverting a distal portion of the tubular mesh.

In an example, a proximal portion of a distal globular mesh or nest can be nested within the concavity of a proximal bowl-shaped mesh or net. In an example, the diameter of a proximal bowl-shaped mesh or net can be larger than the diameter of a distal globular mesh or net. In an example, the diameter of a proximal bowl-shaped mesh or net can be between 5% and 20% larger than the diameter of a distal globular (e.g. spherical and/or cardioid) mesh or net. In an example, the diameter of a proximal bowl-shaped mesh or net can be between 20% and 35% larger than the diameter of a distal globular mesh or net. In an example, the diameter of a proximal bowl-shaped mesh or net can be between 40% and 60% larger than the diameter of a distal globular mesh or net.

In an example, an intrasacular aneurysm occlusion device with a proximal bowl-shaped mesh and a distal globular mesh can be made by: radially-constraining the proximal end of a tubular mesh (e.g. with a ring, band, wire, or string), everting the proximal half of the tubular mesh, and inverting the distal half of the tubular mesh. In another embodiment, an intrasacular aneurysm occlusion device with a proximal bowl-shaped mesh and a distal cardioid mesh can be formed in the following steps: radially-constraining the distal end of a tubular mesh, everting a proximal portion of the tubular mesh in a distal direction, and inverting a distal portion of the tubular mesh in a proximal direction. In an example, an intrasacular aneurysm occlusion device with a proximal bowl-shaped mesh and a distal cardioid-shaped mesh can be made by: radially-constraining the proximal end of a tubular mesh, everting a portion of the tubular mesh, and inverting a portion of the tubular mesh.

In an example, a resilient wider-than-neck (e.g. proximal bowl-shaped) portion and a flexible sac-filling (e.g. distal globular) portion can be different parts of the same continuous structure, with the resilient wider-than-neck (e.g. proximal bowl-shaped) portion comprising a proximal surface of the structure and the flexible sac-filling (e.g. distal globular) portion comprising a distal surface of the structure. In an example, a resilient wider-than-neck (e.g. proximal bowl-shaped) portion of this device and a flexible sac-filling (e.g. distal globular) portion of this device can be proximal and distal portions, respectively, of an intrasacular occlusion device.

In an example, an intrasacular aneurysm occlusion device with a proximal bowl-shaped mesh and a distal globular (e.g. spherical and/or cardioid) mesh can be made by: radially-constraining the distal end of a tubular mesh, everting a proximal portion of the tubular mesh, and inverting a distal portion of the tubular mesh. In one embodiment, an intrasacular aneurysm occlusion device with a proximal bowl-shaped mesh and a distal cardioid mesh can be made by: radially-constraining the proximal end of a tubular mesh, everting the proximal third of the tubular mesh, and inverting the distal third of the tubular mesh. In another example, an intrasacular aneurysm occlusion device with a proximal bowl-shaped mesh or net and a distal globular mesh or net can be formed by: radially-constraining the distal end of a tubular mesh, everting the proximal third of the tubular mesh, and inverting the distal third of the tubular mesh.

In an example, a proximal bowl-shaped mesh or net and a distal globular mesh or net can be continuous. In an example, a proximal bowl-shaped mesh or net and a distal globular mesh or net can be separate parts that are connected together. In an example, a proximal bowl-shaped mesh or net and a distal globular (e.g. spherical and/or cardioid) mesh or net can be parts that created separately and then connected together. In an example, a proximal bowl-shaped mesh or net and a distal globular mesh or net can be separate parts that are connected together at a proximal location. In an example, the proximal end of a distal globular mesh or net can be connected to a proximal bowl-shaped mesh or net. In an example, a central proximal end of a distal globular mesh or net can be connected to the center of a proximal bowl-shaped mesh or net.

In an example, a proximal bowl-shaped mesh and a distal globular (e.g. spherical and/or cardioid) mesh can be made by: radially-constraining the distal end of a tubular mesh, everting a portion of the tubular mesh, and inverting a portion of the tubular mesh. In another example, an intrasacular aneurysm occlusion device with a proximal bowl-shaped mesh and a distal cardioid (e.g. heart-shaped) mesh can be made by: radially-constraining the distal end of a tubular mesh (e.g. with a ring, band, wire, or string), everting the proximal third of the tubular mesh in a distal direction, and inverting the distal third of the tubular mesh in a proximal direction. Alternatively, an intrasacular aneurysm occlusion device with a proximal bowl-shaped mesh or net and a distal globular mesh or net can be made in the following steps: radially-constraining the proximal end of a tubular mesh, inverting the proximal half of the tubular mesh, and everting the distal half of the tubular mesh.

In an example, an intrasacular aneurysm occlusion device with a proximal bowl-shaped mesh and a distal globular mesh can be made in the following steps: radially-constraining the proximal end of a tubular mesh, inverting a portion of the tubular mesh, and everting a portion of the tubular mesh. In one embodiment, an intrasacular aneurysm occlusion device with a proximal bowl-shaped mesh and a distal cardioid mesh can be made by: radially-constraining the proximal end of a tubular mesh (e.g. with a ring, band, wire, or string), inverting the proximal third of the tubular mesh in a distal direction, and everting the distal third of the tubular mesh in a proximal direction. In another embodiment, an intrasacular aneurysm occlusion device with a proximal bowl-shaped mesh and a distal globular (e.g. spherical and/or cardioid) mesh can be made by: radially-constraining the distal end of a tubular mesh, inverting the proximal third of the tubular mesh in a distal direction, and everting the distal third of the tubular mesh in a proximal direction.

In an example, a proximal bowl-shaped mesh or net and a distal globular mesh or net can connected together by a band, ring, or cylinder. In an example, the distal surface of a proximal bowl-shaped mesh or net can be connected to the proximal end of a distal globular mesh or net by a band, ring, or cylinder. In an example, the distal surface of a proximal bowl-shaped mesh or net can be connected to the proximal end of a distal globular mesh or net by adhesion, soldering, crimping, braiding, weaving, tying, or helical threads.

In an example, an intrasacular aneurysm occlusion device with a proximal bowl-shaped mesh and a distal cardioid mesh can be formed in the following steps: radially-constraining the proximal end of a tubular mesh (e.g. with a ring, band, wire, or string), inverting the proximal half of the tubular mesh, and everting the distal half of the tubular mesh over the rest of the tubular mesh. In an example, an intrasacular aneurysm occlusion device with a proximal bowl-shaped mesh or net and a distal globular mesh or net can be made by: radially-constraining the distal end of a tubular mesh, inverting the proximal half of the tubular mesh, and everting the distal half of the tubular mesh over the rest of the tubular mesh. In another example, an intrasacular aneurysm occlusion device with a proximal bowl-shaped mesh and a distal cardioid-shaped mesh can be made by: radially-constraining the proximal end of a tubular mesh, inverting a proximal portion of the tubular mesh, and everting a distal portion of the tubular mesh.

In an example, an intrasacular aneurysm occlusion device with a proximal bowl-shaped mesh or net and a distal globular (e.g. spherical and/or cardioid) mesh or net can be made by: radially-constraining the proximal end of a tubular mesh, everting the proximal third of the tubular mesh, and inverting the distal third of the tubular mesh. In another embodiment, an intrasacular aneurysm occlusion device with a proximal bowl-shaped mesh and a distal cardioid (e.g. heart-shaped) mesh can be formed in the following steps: radially-constraining the distal end of a tubular mesh, everting the proximal half of the tubular mesh in a distal direction, and inverting the distal half of the tubular mesh in a proximal direction. In an example, a proximal bowl-shaped mesh and a distal globular mesh can be made by: radially-constraining the proximal end of a tubular mesh, inverting a proximal portion of the tubular mesh, and everting a distal portion of the tubular mesh.

In an alternative example, a proximal bowl-shaped mesh or net and a distal globular (e.g. spherical and/or cardioid) mesh or net can be non-continuous components which have been created separately and then connected to each other. In an example, a proximal bowl-shaped mesh or net and a distal globular mesh or net can be connected along their central proximal-to-distal axes. In an example, a proximal portion of a distal mesh or net can be attached to a portion of a proximal mesh or net by adhesive, soldering, melting, crimping, riveting, clipping, wiring, tying, or suturing.

In an example, an intrasacular aneurysm occlusion device with a proximal bowl-shaped mesh and a distal globular mesh can be made by: radially-constraining the proximal end of a tubular mesh, inverting the proximal half of the tubular mesh in a distal direction, and everting the distal half of the tubular mesh in a proximal direction. In another example, an intrasacular aneurysm occlusion device with a proximal bowl-shaped mesh and a distal cardioid mesh can be made by: radially-constraining the distal end of a tubular mesh, inverting the proximal half of the tubular mesh, and everting the distal half of the tubular mesh over the rest of the tubular mesh.

In an example, an intrasacular aneurysm occlusion device with a proximal bowl-shaped mesh and a distal cardioid (e.g. heart-shaped) mesh can be formed by: radially-constraining the distal end of a tubular mesh (e.g. with a ring, band, wire, or string), inverting a portion of the tubular mesh, and everting a portion of the tubular mesh. Alternatively, an intrasacular aneurysm occlusion device with a proximal bowl-shaped mesh and a distal cardioid mesh can be made in the following steps: radially-constraining the proximal end of a tubular mesh, inverting the proximal half of the tubular mesh in a distal direction, and everting the distal half of the tubular mesh in a proximal direction. In another example, an intrasacular aneurysm occlusion device with a proximal bowl-shaped mesh and a distal globular mesh can be made by: radially-constraining the distal end of a tubular mesh, inverting the proximal half of the tubular mesh in a distal direction, and everting the distal half of the tubular mesh in a proximal direction.

In an example, a proximal bowl-shaped mesh or net and a distal globular (e.g. spherical and/or cardioid) mesh or net can be continuous. In an example, a proximal bowl-shaped mesh or net and a distal globular mesh or net can be part of the same continuous structure. In an example, a proximal bowl-shaped mesh or net and a distal globular mesh or net can be formed from a single continuous structure. In an example, a proximal bowl-shaped mesh or net and a distal globular mesh or net can be formed from a single tubular mesh or net.

In an example, an intrasacular aneurysm occlusion device with a proximal bowl-shaped mesh and a distal cardioid mesh can be formed in the following steps: radially-constraining the proximal end of a tubular mesh, everting the proximal half of the tubular mesh in a distal direction, and inverting the distal half of the tubular mesh in a proximal direction. In another example, an intrasacular aneurysm occlusion device with a proximal bowl-shaped mesh and a distal globular (e.g. spherical and/or cardioid) mesh can be made by: radially-constraining the distal end of a tubular mesh, everting the proximal half of the tubular mesh in a distal direction, and inverting the distal half of the tubular mesh in a proximal direction.

In one embodiment, an intrasacular aneurysm occlusion device with a proximal bowl-shaped mesh and a distal cardioid-shaped mesh can be formed by: radially-constraining the distal end of a tubular mesh, inverting a proximal portion of the tubular mesh in a distal direction, and everting a distal portion of the tubular mesh in a proximal direction. In an example, an intrasacular aneurysm occlusion device with a proximal bowl-shaped mesh and a distal cardioid (e.g. heart-shaped) mesh can be made in the following steps: radially-constraining the proximal end of a tubular mesh, everting the proximal half of the tubular mesh, and inverting the distal half of the tubular mesh. Alternatively, an intrasacular aneurysm occlusion device with a proximal bowl-shaped mesh and a distal globular mesh can be made by: radially-constraining the distal end of a tubular mesh, everting the proximal half of the tubular mesh, and inverting the distal half of the tubular mesh.

In an example, a proximal bowl-shaped mesh or net and a distal globular mesh or net can be formed by radially-constraining a tubular mesh or net at different longitudinal locations. In an example, a proximal bowl-shaped mesh or net and a distal globular mesh or net can be formed by radially-constraining and inverting a tubular mesh or net at different longitudinal locations. In an example, a proximal bowl-shaped mesh or net and a distal globular mesh or net can be formed by inverting or everting a tubular mesh or net at different longitudinal locations.

In an example, an intrasacular aneurysm occlusion device with a proximal bowl-shaped mesh and a distal globular mesh can be made in the following steps: radially-constraining the proximal end of a tubular mesh, everting the proximal third of the tubular mesh in a distal direction, and inverting the distal third of the tubular mesh in a proximal direction. In an example, an intrasacular aneurysm occlusion device with a proximal bowl-shaped mesh and a distal cardioid mesh can be made by: radially-constraining the distal end of a tubular mesh, everting the proximal third of the tubular mesh, and inverting the distal third of the tubular mesh. In another example, an intrasacular aneurysm occlusion device with a proximal bowl-shaped mesh and a distal globular (e.g. spherical and/or cardioid) mesh can be formed by: radially-constraining the proximal end of a tubular mesh, everting a proximal portion of the tubular mesh in a distal direction, and inverting a distal portion of the tubular mesh in a proximal direction.

In one embodiment, an intrasacular aneurysm occlusion device with a proximal bowl-shaped mesh and a distal globular mesh can be made by: radially-constraining the distal end of a tubular mesh, inverting a proximal portion of the tubular mesh, and everting a distal portion of the tubular mesh. In another embodiment, an intrasacular aneurysm occlusion device with a proximal bowl-shaped mesh and a distal globular (e.g. spherical and/or cardioid) mesh can be formed in the following steps: radially-constraining the proximal end of a tubular mesh, everting the proximal half of the tubular mesh in a distal direction, and inverting the distal half of the tubular mesh in a proximal direction. In an example, an intrasacular aneurysm occlusion device with a proximal bowl-shaped mesh and a distal cardioid mesh can be made by: radially-constraining the distal end of a tubular mesh (e.g. with a ring, band, wire, or string), everting the proximal half of the tubular mesh, and inverting the distal half of the tubular mesh.

In an example, a proximal bowl-shaped mesh or net and a distal globular mesh or net can be formed from a single continuous mesh or net. In an example, a proximal bowl-shaped mesh or net and a distal globular mesh or net can be formed by radially-constraining, inverting, and/or everting a single continuous mesh or net. In an example, a proximal bowl-shaped mesh or net and a distal globular mesh or net can be formed by radially-constraining, inverting, and/or everting a single continuous tubular mesh or net. In an example, a proximal bowl-shaped mesh or net and a distal globular mesh or net can be formed by radially-constraining, inverting, and/or everting a single continuous tubular mesh or net at multiple locations along the length of the tubular mesh or net.

In an example, an intrasacular aneurysm occlusion device with a proximal bowl-shaped mesh or net and a distal globular (e.g. spherical and/or cardioid) mesh or net can be made by: radially-constraining the proximal end of a tubular mesh, everting a portion of the tubular mesh, and inverting a portion of the tubular mesh. In one embodiment, an intrasacular aneurysm occlusion device with a proximal bowl-shaped mesh and a distal cardioid mesh can be made by: radially-constraining the proximal end of a tubular mesh, everting the proximal third of the tubular mesh in a distal direction, and inverting the distal third of the tubular mesh in a proximal direction. In an example, an intrasacular aneurysm occlusion device with a proximal bowl-shaped mesh and a distal globular mesh can be made in the following steps: radially-constraining the distal end of a tubular mesh (e.g. with a ring, band, wire, or string), everting the proximal third of the tubular mesh in a distal direction, and inverting the distal third of the tubular mesh in a proximal direction.

In one embodiment, an intrasacular aneurysm occlusion device with a proximal bowl-shaped mesh or net and a distal globular mesh or net can be made by: radially-constraining the proximal end of a tubular mesh, inverting the proximal third of the tubular mesh in a distal direction, and everting the distal third of the tubular mesh in a proximal direction. In an example, an intrasacular aneurysm occlusion device with a proximal bowl-shaped mesh and a distal cardioid (e.g. heart-shaped) mesh can be made by: radially-constraining the distal end of a tubular mesh (e.g. with a ring, band, wire, or string), inverting the proximal third of the tubular mesh, and everting the distal third of the tubular mesh.

In an alternative example, a proximal bowl-shaped mesh or net and a distal globular mesh or net can be continuous. In an alternative example, a proximal bowl-shaped mesh or net and a distal globular (e.g. spherical and/or cardioid) mesh or net can be formed from the same continuous structure. In an example, a proximal bowl-shaped mesh or net and a distal globular mesh or net can be two portions which are formed from a continuous component (e.g. a continuous tubular mesh or braid which is radially-constrained).

In one embodiment, an intrasacular aneurysm occlusion device with a proximal bowl-shaped mesh and a distal cardioid-shaped mesh can be made by: radially-constraining the distal end of a tubular mesh (e.g. with a ring, band, wire, or string), everting a proximal portion of the tubular mesh, and inverting a distal portion of the tubular mesh. In another embodiment, an intrasacular aneurysm occlusion device with a proximal bowl-shaped mesh and a distal globular mesh can be formed in the following steps: radially-constraining the proximal end of a tubular mesh, inverting a proximal portion of the tubular mesh in a distal direction, and everting a distal portion of the tubular mesh in a proximal direction. In another example, an intrasacular aneurysm occlusion device with a proximal bowl-shaped mesh and a distal cardioid mesh can be made by: radially-constraining the distal end of a tubular mesh, inverting a proximal portion of the tubular mesh, and everting a distal portion of the tubular mesh.

In one embodiment, an intrasacular aneurysm occlusion device with a proximal bowl-shaped mesh and a distal cardioid (e.g. heart-shaped) mesh can be made by: radially-constraining the proximal end of a tubular mesh (e.g. with a ring, band, wire, or string), inverting a proximal portion of the tubular mesh in a distal direction, and everting a distal portion of the tubular mesh in a proximal direction. In another embodiment, an intrasacular aneurysm occlusion device with a proximal bowl-shaped mesh and a distal globular (e.g. spherical and/or cardioid) mesh can be made in the following steps: radially-constraining the distal end of a tubular mesh, inverting a proximal portion of the tubular mesh in a distal direction, and everting a distal portion of the tubular mesh over the rest of the tubular mesh in a proximal direction.

In an example, there can be a hyperbolic, hourglass, and/or funnel shaped opening where a proximal bowl-shaped mesh or net is connected to a distal globular mesh or net. In an example, the interface between a proximal bowl-shaped mesh or net and a distal globular mesh or net can have a hyperbolic, hourglass, and/or funnel shape. In an example, the interior surface of the connection between a proximal bowl-shaped mesh or net and a distal globular mesh or net can have a hyperbolic, hourglass, and/or funnel shape. In an example, there can be a hyperbolic, hourglass, and/or funnel shaped opening where a proximal bowl-shaped mesh or net is connected to a distal globular mesh or net, wherein embolic members and/or material is inserted through this opening into the distal globular mesh or net.

In an example, a proximal bowl-shaped mesh or net and a distal globular mesh or net can share a common proximal perimeter. In an example, the proximal bowl-shaped mesh or net can overlap the proximal half of the distal globular mesh or net. In an example, the proximal bowl-shaped mesh or net can be the proximal half of the distal globular mesh or net. In an example, the proximal bowl-shaped mesh or net can be a proximal portion (e.g. proximal third or half) of the distal globular mesh or net.

In an example, a proximal bowl-shaped mesh or net and a distal globular mesh or net can have the same levels of porosity, thickness, elasticity, flexibility, durometer, density, compliance, conformability, and/or malleability. In an example, a proximal bowl-shaped mesh or net and a distal globular mesh or net can have a different levels of porosity, thickness, elasticity, flexibility, durometer, density, compliance, conformability, and/or malleability. In an example, a proximal bowl-shaped mesh or net can have a first level of porosity, thickness, elasticity, flexibility, durometer, density, compliance, conformability, and/or malleability; and a distal globular mesh or net can have a second level of porosity, thickness, elasticity, flexibility, durometer, density, compliance, conformability, and/or malleability; wherein the first level is greater than the second level.

In an example, a proximal bowl-shaped mesh or net can be more compliant than a distal globular mesh or net. In an example, a proximal bowl-shaped mesh or net can be less elastic than a distal globular mesh or net. In an example, a proximal bowl-shaped mesh or net can be more conformable than a distal globular mesh or net. In an example, a proximal bowl-shaped mesh or net can be more elastic than a distal globular mesh or net. In an example, a proximal bowl-shaped mesh or net can be more flexible than a distal globular mesh or net. In an example, a proximal bowl-shaped mesh or net can be made from a metal and a distal globular mesh or net can be made from a polymer.

In an example, a proximal bowl-shaped mesh or net can be more malleable than a distal globular mesh or net. In an example, a proximal bowl-shaped mesh or net can be more porous than a distal globular mesh or net. In an example, a proximal bowl-shaped mesh or net can be thicker than a distal globular mesh or net. In an example, a proximal bowl-shaped mesh or net can have a larger hole size than that of a distal globular mesh or net. In an example, a proximal bowl-shaped mesh or net can have a higher durometer level than a distal globular mesh or net. In an example, a proximal bowl-shaped mesh or net can be made from a polymer and a distal globular mesh or net can be made from a metal.

In an example, a proximal bowl-shaped mesh or net can be made with a greater percentage of polymer material than a distal globular mesh or net. In an example, a proximal bowl-shaped mesh or net can be less compliant than a distal globular mesh or net. In an example, a proximal bowl-shaped mesh or net can have a lower durometer level than a distal globular mesh or net. In an example, a proximal bowl-shaped mesh or net can be less flexible than a distal globular mesh or net. In an example, a proximal bowl-shaped mesh or net can be made with a lower percentage of polymer material than a distal globular mesh or net.

In an example, a proximal bowl-shaped mesh or net can be less malleable than a distal globular mesh or net. In an example, a proximal bowl-shaped mesh or net can be made from a different material than a distal globular mesh or net. In an example, a proximal bowl-shaped mesh or net can have a first level of porosity, thickness, elasticity, flexibility, durometer, density, compliance, conformability, and/or malleability; and a distal globular mesh or net can have a second level of porosity, thickness, elasticity, flexibility, durometer, density, compliance, conformability, and/or malleability; wherein the first level is less than the second level.

In an example, a proximal bowl-shaped mesh or net can be less conformable than a distal globular mesh or net. In an example, a proximal bowl-shaped mesh or net can have a smaller hole size than that of distal globular mesh or net. In an example, a proximal bowl-shaped mesh or net can have a different hole shape than a distal globular mesh or net. In an example, a proximal bowl-shaped mesh or net can be less porous than a distal globular mesh or net. In an example, a proximal bowl-shaped mesh or net can be thinner than a distal globular mesh or net.

In an example, a proximal bowl-shaped mesh or net can be more dense than a distal globular mesh or net. In an example, a proximal bowl-shaped mesh or net can have a higher durometer level than a distal globular mesh or net. In an example, a proximal bowl-shaped mesh or net can be less porous than a distal globular mesh or net. In an example, a proximal bowl-shaped mesh or net can be less elastic than a distal globular mesh or net. In an example, a proximal bowl-shaped mesh or net can be thicker than a distal globular mesh or net.

In an example, a proximal bowl-shaped mesh or net can be at least 50% more dense than a distal globular mesh or net. In an example, a proximal bowl-shaped mesh or net can have a higher durometer level which is at least 50% greater than that of a distal globular mesh or net. In an example, a proximal bowl-shaped mesh or net can be at least 50% less porous than a distal globular mesh or net. In an example, a proximal bowl-shaped mesh or net can be at least 50% less elastic than a distal globular mesh or net. In an example, a proximal bowl-shaped mesh or net can be at least 50% thicker than a distal globular mesh or net.

In an example, the diameter of a proximal bowl-shaped mesh or net can be greater than the diameter of a distal globular mesh or net. In an example, the diameter of a proximal bowl-shaped mesh or net can be between 5% and 30% greater than the diameter of a distal globular mesh or net. In an example, the diameter of a proximal bowl-shaped mesh or net can be between 10% and 50% greater than the diameter of a distal globular mesh or net.

In an example, the height of a distal globular mesh or net can be greater than the height of a proximal bowl-shaped mesh or net. In an example, the height of a distal globular mesh or net can be between 20% and 50% greater than the height of a proximal bowl-shaped mesh or net. In an example, the height of a distal globular mesh or net can be between 50% and 150% greater than the height of a proximal bowl-shaped mesh or net. In an example, the height of a distal globular mesh or net can be at least twice the height of a proximal bowl-shaped mesh or net.

In an example, there can be a gap (e.g. gap or space) between most of the surface of a proximal bowl-shaped mesh or net and most of the surface of a distal globular mesh or net. In an example, this gap (e.g. gap or space) can have a uniform width. In an example, the distal surface of a proximal bowl-shaped mesh or net can be concave; and the proximal surface of a distal globular mesh or net can be partly convex and partly concave. In an example, this gap (e.g. gap or space) can be greater away from the center of the proximal bowl-shaped mesh or net. In an example, this gap (e.g. gap or space) can be greater toward the center of the proximal bowl-shaped mesh or net. In an example, this gap (e.g. gap or space) can be smaller away from the center of the proximal bowl-shaped mesh or net.

In an example, the distal surface of a proximal bowl-shaped mesh or net can be concave and the proximal surface of a distal globular mesh or net can be convex. In an example, the distal surface of a proximal bowl-shaped mesh or net can be partly concave and partly convex; and the proximal surface of a distal globular mesh or net can convex. In an example, this gap (e.g. gap or space) can be smaller toward the center of the proximal bowl-shaped mesh or net. In an example, the proximal surface of a distal globular mesh or net can be partly concave and partly convex. In an example, the distal surface of a proximal bowl-shaped mesh or net can be partly concave and partly convex. In an example, the proximal surface of a proximal bowl-shaped mesh or net can be partly concave and partly convex.

In an example, the distance between a proximal bowl-shaped mesh or net and a distal globular mesh or net can be remotely adjusted (e.g. increased or decreased) by a device operator by activating an electromagnet. In an example, the distance between a proximal bowl-shaped mesh or net and a distal globular mesh or net can be remotely adjusted (e.g. increased or decreased) by a device operator by applying electrical energy. In an example, the distance between a proximal bowl-shaped mesh or net and a distal globular mesh or net can be remotely adjusted (e.g. increased or decreased) by a device operator by activating an electromagnetic field.

In an example, there can be a gap (e.g. gap or space) between most of the surface of a proximal bowl-shaped mesh or net and most of the surface of a distal globular mesh or net, wherein this gap can be remotely adjusted by the operator of the device. In an example, the distance between a proximal bowl-shaped mesh or net and a distal globular mesh or net can be remotely adjusted (e.g. increased or decreased) by a device operator. In an example, the distance between a proximal bowl-shaped mesh or net and a distal globular mesh or net can be remotely adjusted (e.g. increased or decreased) by a device operator by pulling or pushing a longitudinal member (e.g. a wire, filament, coil, or string). In an example, the distance between a proximal bowl-shaped mesh or net and a distal globular mesh or net can be remotely adjusted (e.g. increased or decreased) by a device operator by rotating a longitudinal member (e.g. a wire, filament, coil, or string).

In an example, a proximal bowl-shaped mesh or net and a distal globular mesh or net can share a common lumen (e.g. opening or hole) through which embolic members or material (e.g. coils, hydrogels, microsponges, beads, string-of-pearls threaded embolic components, or embolizing liquid) is inserted through the proximal bowl-shaped mesh or net into the distal globular mesh or net. In an example, a proximal bowl-shaped mesh or net and a distal globular mesh or net can share a common central lumen (e.g. opening or hole) through which embolic members or material (e.g. coils, hydrogels, microsponges, beads, string-of-pearls threaded embolic components, or embolizing liquid) is inserted through the proximal bowl-shaped mesh or net into the distal globular mesh or net.

In an example, a proximal bowl-shaped mesh or net and a distal globular mesh or net can be coaxial, sharing a common central lumen (e.g. opening or hole) through which embolic members or material (e.g. coils, hydrogels, microsponges, beads, string-of-pearls threaded embolic components, or embolizing liquid) is inserted through the proximal bowl-shaped mesh or net into the distal globular mesh or net. In an example, there can be a central opening (e.g. tube or pathway) through a proximal bowl-shaped mesh or net and a distal globular mesh or net through which embolic members or material (e.g. coils, hydrogels, microsponges, beads, string-of-pearls threaded embolic components, or embolizing liquid) is inserted through into the distal globular mesh or net.

In an example, a distal globular mesh or net and a proximal bowl-shaped mesh or net can be inserted into an aneurysm sac at substantially the same time. In an example, a distal globular mesh or net can be inserted into an aneurysm sac before a proximal bowl-shaped mesh or net is inserted into the aneurysm sac. In an example, a distal globular mesh or net and a proximal bowl-shaped mesh or net can be expanded in an aneurysm sac at substantially the same time. In an example, a proximal bowl-shaped mesh or net can be expanded in an aneurysm sac before a distal globular mesh or net is expanded in the aneurysm sac. In an example, a distal globular mesh or net and a proximal bowl-shaped mesh or net can be moved closer to each other (e.g. into a nested configuration) after they have been inserted into an aneurysm sac. In an example, a distal globular mesh or net and a proximal bowl-shaped mesh or net can be moved closer to each other (e.g. into a nested configuration) after they have been inserted into an aneurysm sac and expanded in the aneurysm sac.

In an example, a proximal portion (e.g. half) of a tubular mesh from which proximal and distal mesh portions are formed can have a higher durometer level than a distal portion (e.g. half) of this tubular mesh. In an example, a proximal portion (e.g. half) of a tubular mesh from which proximal and distal mesh portions are formed can be less porous than a distal portion (e.g. half) of this tubular mesh. In an example, a proximal portion (e.g. half) of a tubular mesh from which proximal and distal mesh portions are formed can have a different mesh hole shape than a distal portion (e.g. half) of this tubular mesh. In an example, a proximal portion (e.g. half) of a tubular mesh from which proximal and distal mesh portions are formed can be more elastic than a distal portion (e.g. half) of this tubular mesh.

In an example, a proximal portion (e.g. half) of a tubular mesh from which proximal and distal mesh portions are formed can have a higher compliance level than a distal portion (e.g. half) of this tubular mesh. In an example, a proximal portion (e.g. half) of a tubular mesh from which proximal and distal mesh portions are formed can be less elastic than a distal portion (e.g. half) of this tubular mesh. In an example, a proximal portion (e.g. half) of a tubular mesh from which proximal and distal mesh portions are formed can be more flexible than a distal portion (e.g. half) of this tubular mesh.

In an example, a proximal portion (e.g. half) of a tubular mesh from which proximal and distal mesh portions are formed can be less flexible than a distal portion (e.g. half) of this tubular mesh. In an example, a proximal portion (e.g. half) of a tubular mesh from which proximal and distal mesh portions are formed can be less conformable than a distal portion (e.g. half) of this tubular mesh. In an example, a proximal portion (e.g. half) of a tubular mesh from which proximal and distal mesh portions are formed can be made from a different material than a distal portion (e.g. half) of this tubular mesh. In an example, a proximal portion (e.g. half) of a tubular mesh from which proximal and distal mesh portions are formed can be made from a metal and the distal portion (e.g. half) of this tubular mesh can be made from a polymer.

In an example, a proximal portion (e.g. half) of a tubular mesh from which proximal and distal mesh portions are formed can be made from a polymer and the distal portion (e.g. half) of this tubular mesh can be made from a metal. In an example, a proximal portion (e.g. half) of a tubular mesh from which proximal and distal mesh portions are formed can be thicker than a distal portion (e.g. half) of this tubular mesh. In an example, a proximal portion (e.g. half) of a tubular mesh from which proximal and distal mesh portions are formed can be tapered. In an example, a proximal portion (e.g. half) of a tubular mesh from which proximal and distal mesh portions are formed can be denser than a distal portion (e.g. half) of this tubular mesh. In an example, a proximal portion (e.g. half) of a tubular mesh from which proximal and distal mesh portions are formed can be less dense than a distal portion (e.g. half) of this tubular mesh.

In an example, a proximal portion (e.g. half) of a tubular mesh from which proximal and distal mesh portions are formed can have a larger circumference than a distal portion (e.g. half) of this tubular mesh. In an example, a proximal portion (e.g. half) of a tubular mesh from which proximal and distal mesh portions are formed can be thinner than a distal portion (e.g. half) of this tubular mesh. In an example, a proximal portion (e.g. half) of a tubular mesh from which proximal and distal mesh portions are formed can have a lower compliance level than a distal portion (e.g. half) of this tubular mesh.

In an example, a proximal portion (e.g. half) of a tubular mesh from which proximal and distal mesh portions are formed can have a lower durometer level than a distal portion (e.g. half) of this tubular mesh. In an example, a proximal portion (e.g. half) of a tubular mesh from which proximal and distal mesh portions are formed can be more compliant than a distal portion (e.g. half) of this tubular mesh. In an example, a proximal portion (e.g. half) of a tubular mesh from which proximal and distal mesh portions are formed can be more conformable than a distal portion (e.g. half) of this tubular mesh. In an example, a proximal portion (e.g. half) of a tubular mesh from which proximal and distal mesh portions are formed can be more porous than a distal portion (e.g. half) of this tubular mesh.

In an example, a proximal portion (e.g. half) of a tubular mesh from which proximal and distal mesh portions are formed can have a smaller circumference than a distal portion (e.g. half) of this tubular mesh. In an example, a proximal portion (e.g. half) of a tubular mesh from which proximal and distal mesh portions are formed can have smaller mesh holes size than a distal portion (e.g. half) of this tubular mesh. In an example, a proximal portion (e.g. half) of a tubular mesh from which proximal and distal mesh portions are formed can be less compliant than a distal portion (e.g. half) of this tubular mesh. In an example, a proximal portion (e.g. half) of a tubular mesh from which proximal and distal mesh portions are formed can have larger mesh holes size than a distal portion (e.g. half) of this tubular mesh.

In an example, a tubular mesh from which proximal and distal meshes are formed can be radially constrained by a ring, band, or cylinder. In an example, a tubular mesh from which proximal and distal meshes are formed can be radially constrained by a wire, cord, or string. In an example, a tubular mesh from which proximal and distal meshes are formed can be radially constrained by an annular member. In an example, a tubular mesh from which proximal and distal meshes are formed can be radially constrained at two or more locations. In an example, a tubular mesh from which proximal and distal meshes are formed can be radially constrained at both ends and also a middle location.

In an example, a proximal bowl-shaped mesh or net can be connected (e.g. connected or attached) to a distal globular mesh or net by a connector. In an example, the proximal center of a proximal bowl-shaped mesh or net can be connected (e.g. connected or attached) to the proximal center of a distal globular mesh or net by a connector. In an example, a proximal bowl-shaped mesh or net can be connected (e.g. connected or attached) to the proximal center of a distal globular mesh or net by a connector on their shared proximal-to-distal axis. In an example, a connector can be selected from the group consisting of: ring, tube, chain, washer, string, wire, rivet, snap, annular member, clamp, thread, clip, helical fastener, band, and cylinder. In an example, a connector can have a circular, toroidal, or cylindrical shape. In an example, a connector can be a ring, band, tube, or threaded cylinder.

In an example, a connector which connected a proximal bowl-shaped mesh or net and a distal globular mesh or net can comprise an inner portion and an outer portion, wherein the ends of one or more of the meshes or nets are held (e.g. pinched, crimped, bound, soldered, or glued) between the inner portion and the outer portion. In an example, a connector which connected a proximal bowl-shaped mesh or net and a distal globular mesh or net can two coaxial portions, an inner portion and an outer portion, wherein the ends of one or more of the meshes or nets are held (e.g. pinched, crimped, bound, soldered, or glued) between the inner portion and the outer portion.

In an example, a connector which connected a proximal bowl-shaped mesh or net and a distal globular mesh or net can two coaxial cylindrical portions, an inner cylindrical portion and an outer cylindrical portion, wherein the ends of one or more of the meshes or nets are held (e.g. pinched, crimped, bound, soldered, or glued) between the inner and outer cylindrical portion. In an example, a connector which connected a proximal bowl-shaped mesh or net and a distal globular mesh or net can two coaxial rings, an inner ring and an outer ring, wherein the ends of one or more of the meshes or nets are held (e.g. pinched, crimped, bound, soldered, or glued) between the inner and outer rings.

In an example, a connector which connected a proximal bowl-shaped mesh or net and a distal globular mesh or net can comprise an inner portion and an outer portion, wherein the ends of one or more of the meshes or nets are held (e.g. pinched, crimped, bound, soldered, or glued) between the inner portion and outer portions, and wherein embolic members or material is inserted through the inner portion. In an example, a connector which connected a proximal bowl-shaped mesh or net and a distal globular mesh or net can two coaxial portions, an inner portion and an outer portion, wherein the ends of one or more of the meshes or nets are held (e.g. pinched, crimped, bound, soldered, or glued) between the inner and outer portions, and wherein embolic members or material is inserted through the inner portion. In an example, a connector can comprise two coaxial members (e.g. rings and/or cylinders), an inner member and an outer member, wherein at least one of the nets or meshes are pinched (e.g. held in place by compression) between the inner member and the outer member.

In an example, a connector which connected a proximal bowl-shaped mesh or net and a distal globular mesh or net can two coaxial cylindrical portions, an inner cylindrical portion and an outer cylindrical portion, wherein the ends of one or more of the meshes or nets are held (e.g. pinched, crimped, bound, soldered, or glued) between the inner and outer cylindrical portion, and wherein embolic members or material is inserted through the inner portion. In an example, a connector which connected a proximal bowl-shaped mesh or net and a distal globular mesh or net can two coaxial rings, an inner ring and an outer ring, wherein the ends of one or more of the meshes or nets are held (e.g. pinched, crimped, bound, soldered, or glued) between the inner and outer rings, and wherein embolic members or material is inserted through the inner rings.

In an example, a proximal bowl-shaped mesh or net and a distal globular mesh or net can be directly attached to each other. In an example, a proximal bowl-shaped mesh or net and a distal globular mesh or net can be directly attached to each other by crimping them together, soldering them together, tying them together, welding them together, binding them together, gluing then together, melting them together, screwing them together, clipping them together, or riveting them together.

In an example, embolic members and/or material can be inserted into a distal globular mesh or net. In an example, embolic members and/or material can be inserted through an opening in a proximal bowl-shaped mesh or net into a distal globular mesh or net. In an example, embolic members and/or material can be inserted through an opening in a proximal bowl-shaped mesh or net into an aneurysm sac. In an example, embolic members and/or material can be inserted through an opening in a distal globular mesh or net into the mesh or net. In an example, embolic members and/or material can be inserted through an opening in a proximal bowl-shaped mesh or net into a distal globular mesh or net, where accumulation of the embolic members and/or material causes the distal globular mesh or net to (further) expand. In an example, embolic members and/or material can be inserted through an opening in a proximal bowl-shaped mesh or net into a distal globular mesh or net, where accumulation of the embolic members and/or material causes the distal globular mesh or net to expand and conform to the contours of an irregularly-shaped aneurysm sac.

In an example, embolic members and/or material can be selected from the group consisting of: microsponges, sponge balls, beads, coils, hydrogels, balls, congealing liquid or gel, filaments, foam, foam pieces, gel balls, gel polyhedrons, microspheres, ribbons, and string-of-pearl embolics. In an example, a string-of-pearls embodiment of embolic members can be a connected longitudinal sequence of microsponges, sponge balls, beads, coils, hydrogels, balls, foam pieces, gel balls, gel polyhedrons, or microspheres. In an example, a string-of-pearls embodiment of embolic members can be a longitudinal sequence of microsponges, sponge balls, beads, coils, hydrogels, balls, foam pieces, gel balls, gel polyhedrons, or microspheres connected by a string, suture, thread, yarn, filament, or wire.

In an example, embolic members can have shapes selected from the group consisting of: spherical, ball shaped, ellipsoidal, globular, ovaloid shaped, ovoid, pair shaped, prolate spherical, and apple shaped. In an example, embolic members can have shapes selected from the group consisting of: hour-glass shaped, pancake shaped, peanut shaped, ring shaped, and toroidal. In an example, embolic members can have shapes selected from the group consisting of: helical, ribbon shaped, sinusoidal, and undulating. In an example, embolic members can have shapes selected from the group consisting of: barrel shaped, disk shaped, polyhedrons comprised of hexagonal surfaces, polyhedrons comprised of quadrilateral surfaces, polyhedrons comprised of triangular surfaces, and pyramid shaped.

In an example, differently-sized embolic members can be used. In an example two or more different sizes of embolic members can be inserted into a mesh or net to occlude an aneurysm. In an example, it may be advantageous to first fill a mesh or net with larger embolic members and then continue filling the mesh or net with smaller embolic members. In another example, it may be advantageous to first fill a mesh or net with smaller embolic members and then continue filling the mesh or net with larger embolic members. In an example, there can be a first average size of embolic members which are inserted into the mesh or net at a first time, a second average size of embolic members which are inserted into the mesh or net at a second time, and the second average size can be greater than the first average size. In an example, there can be a first average size of embolic members which are inserted into the mesh or net at a first time, a second average size of embolic members which are inserted into the mesh or net at a second time, and the second average size can be less than the first average size.

In an example, embolic members which are inserted into a mesh or net can be soft and compressible. In an example, embolic members which are inserted into a mesh or net can have a durometer less than 50. In an example, embolic members which are inserted into a mesh or net can have an average durometer within the range of 10 to 30. In an example, embolic members which are inserted into a mesh or net can have an average durometer within the range of 25 to 50. In an example, embolic members which are inserted into a mesh or net can have an average durometer which is less than 70. In an example, embolic members can have a Shore OO value, indicative of softness or hardness, within a range of 5 to about 50.

In an example, a plurality of embolic members can be delivered into a mesh or net in a linear (longitudinal) array or series of inter-connected embolic members. In an example, a plurality of embolic members can be delivered into a mesh or net in a linear (longitudinal) array of connected embolic members, wherein this linear array can be cut, separated, and/or detached in situ (in a remote manner) at one or more selected locations by the user of the device in order to deliver a selected quantity, length, or volume or embolic members. In an example, a plurality of embolic members can be delivered into a mesh or net in a planar array of inter-connected embolic members. In an example, a plurality of embolic members can be delivered into a mesh or net in a three-dimensional array of inter-connected embolic members.

In an example, embolic members which are inserted into a mesh or net at a first time can have first shapes, embolic members which are inserted into a mesh or net at a second time can have second shapes, and the second shape can be different than the first shape. In an example, embolic members which are inserted into a mesh or net at a first time can be made with a first (combination of) material, embolic members which are inserted into a mesh or net at a second time can be made with a second (combination of) material, and the second (combination of) material can be different from the first (combination of) material. In an example, embolic members which are inserted into a mesh or net at a first time can be made with a first (combination of) material, embolic members which are inserted into a mesh or net at a second time can be made with a second (combination of) material, and the second (combination of) material can be more flexible, elastic, and/or compliant than the first (combination of) material.

In an example, embolic members which are inserted into a mesh or net at a first time can be made with a first (combination of) material, embolic members which are inserted into a mesh or net at a second time can be made with a second (combination of) material, and the second (combination of) material can have a lower durometer than the first (combination of) material. In an example, embolic members which are inserted into a mesh or net at a first time can be made with a first (combination of) material, embolic members which are inserted into a mesh or net at a second time can be made with a second (combination of) material, and the second (combination of) material can be less flexible, elastic, and/or compliant than the first (combination of) material. In an example, embolic members which are inserted into a mesh or net at a first time can be made with a first (combination of) material, embolic members which are inserted into a mesh or net at a second time can be made with a second (combination of) material, and the second (combination of) material can have a higher durometer than the first (combination of) material.

In an example, there can be a first average durometer of embolic members which are inserted into a mesh or net at a first time and a second average durometer of embolic members which are inserted into a mesh or net at a second time, wherein the second average durometer is greater than the first average durometer. In an example, there can be a first average durometer of embolic members which are inserted into a mesh or net at a first time and a second average durometer of embolic members which are inserted into a mesh or net at a second time, wherein the second average durometer is less than the first average durometer. In an example, embolic members can differ among themselves with respect to one or more characteristics selected from the group consisting of: porosity, shape, size, material, composition, coating, radiopacity, strength, stiffness, and type.

In an example, embolic members which are inserted into a mesh or net can comprise “string of pearls” embolic members (e.g. a longitudinal series of embolic members which are connected by a string, wire, filament, chain, or other flexible longitudinal member). In an example, embolic members in a “string of pearls” series can be equally spaced (e.g. separated from each other by the same distance). In an example, embolic members in a “string of pearls” series can be unequally spaced (e.g. separated from each other by the same distance). In an example, embolic members in a “string of pearls” series can be progressively closer to each other as one moves along the series in a proximal-to-distal direction. In an example, embolic members in a “string of pearls” series can be progressively farther from each other as one moves along the series in a proximal-to-distal direction.

In an example, embolic members in a “string of pearls” series can all be the same size. In an example, embolic members in a “string of pearls” series can have different sizes. In an example, the size of embolic members in a “string of pearls” series can increase as one moves along the series in a proximal-to-distal direction. In an example, the size of embolic members in a “string of pearls” series can decrease as one moves along the series in a proximal-to-distal direction.

In an example, embolic members in a “string of pearls” series can all have the same durometer level. In an example, embolic members in a “string of pearls” series can have different durometer levels. In an example, the durometer level of embolic members in a “string of pearls” series can increase as one moves along the series in a proximal-to-distal direction. In an example, the durometer level of embolic members in a “string of pearls” series can decrease as one moves along the series in a proximal-to-distal direction. In an example, the porosity of embolic members in a “string of pearls” series can increase as one moves along the series. In an example, the stiffness of embolic members in a “string of pearls” series can increase as one moves along the series. In an example, the shapes of embolic members in a “string of pearls” series can change as one moves along the series.

In an example, embolic members which are inserted into a mesh or net can expand in size within the mesh or net. In an example, embolic members can have a first (average) size while being delivered to an aneurysm sac via a catheter and a second (average) size after expansion within the aneurysm sac, wherein the second (average) size is 10% to 50% larger than the first (average) size. In an example, embolic members can have a first (average) size while being delivered to an aneurysm sac via a catheter and a second (average) size after expansion within the aneurysm sac, wherein the second (average) size is 40% to 100% larger than the first (average) size. In an example, embolic members can have a first (average) size while being delivered to an aneurysm sac via a catheter and a second (average) size after expansion within the aneurysm sac, wherein the second (average) size is more than twice the first (average) size.

In an example, embolic members can self-expand within a mesh or net after they are released from a delivery catheter. In an example, embolic members can swell upon hydration from interaction with blood or other body fluid. In an example, embolic members can be expanded within the mesh or net by one or more mechanisms selected from the group consisting of: expansion due to interaction with body fluid; expansion due to application of thermal energy; expansion due to exposure to a chemical agent; and expansion due to exposure to light energy. In an example, embolics can expand by a factor of 2-5 times. In an example, embolics can expand by a factor of 4-10 times. In an example, embolics can expand by a factor of more than 10 times. In an example, embolic members can expand to a sufficiently-large size that they cannot escape from the mesh or net after insertion into the mesh or net.

In an example, embolic members and/or material can comprise nitinol, nickel-titanium alloy, titanium, tungsten, zirconium alloy, stainless steel, cobalt chromium alloy, platinum, gold, palladium, and/or tantalum. In an example, embolic members and/or material can comprise collagen, silk, fibrin, fibrinogen, fibronectin, collagen glycosaminoglycan, small intestinal submucosa, carboxy methyl cellulose, chitin, and/or gelatin.

In an example, embolic members and/or material can comprise polyvinyl alcohol (PVA), polydimethylsiloxane (PDMS), vinyl stearate, hydrogel, divinylbenzene, polytetrafluoroethylene (PTFE), hyaluronic acid, polypropylene, ethylene glycol, polyether block amide (PEBA), alginate, ethylene glycol dimethylmathacrylate, polyetherether ketone (PEEK), ethylene vinyl acetate, polyethylene, ethylene vinyl alcohol (EVA), polyethylene glycol (PEG), polyacrylic acid, polyethylene terephthalate (PET), polyglycolic acid (PGA), polylactic acid (PLA), latex, hydroxyethylmethacrylate, polyurethane (PU), polyamides, copolymer, polycarbonate urethane (PCU), urethane, acetate, methlymethacrylate, polyvinyl pyrrolidone (PVP), alginic acid, elastomeric polymer, polyester, nylon, thermoplastic elastomer, hydrocarbon polymer, polysaccharides, acrylic, methylcellulose, silicone-based polymer, polyolefin, and/or poly-N-acetylglucosamine (PNAG).

In an example, embolic members and/or material can be delivered through a catheter to an aneurysm sac. In an example, embolic members and/or material can be pushed through a catheter into an aneurysm sac. In an example, embolic members and/or material can be dispensed out of a catheter into an aneurysm sac by a mechanism selected from the group consisting of: a conveyor belt, a flow of liquid or gel, fluid pressure, a hydraulic mechanism, a magnetic field, a moving chain, a piston mechanism, a pusher rod or plunger, a pusher wire, a rotating helical member (e.g. Archimedes' screw), and a telescoping mechanism.

In an example, there can be an opening (e.g. opening, hole, lumen, channel, or tube) in a proximal bowl-shaped mesh or net through which embolic members or material is inserted, wherein there is also a closure mechanism which closes this opening. In an example, a device operator can remotely close this closure mechanism after embolic members or material has been inserted through the opening. In an example, an opening and closure mechanism can be centrally-located in a proximal bowl-shaped mesh or net. In an example, an opening and closure mechanism can be on a central proximal-to-distal longitudinal axis of a proximal bowl-shaped mesh or net. In an example, an opening and closure mechanism can be part of a connector which connects a proximal bowl-shaped mesh or net with a distal globular mesh or net.

In an example, a closure mechanism can be selected from the group consisting of: a solenoid, a tri-leaflet valve, a helical component which is rotated, a rotatable valve, an elastic band, ring, or loop, an electric detachment mechanism, a piston, two openings which can be selectively aligned or misaligned, a plug, a sliding cover, a bi-leaflet valve, a leaflet valve, a spring-loaded lid, an electromagnet, a one-way valve, a filament loop, and a multi-petal leaflet valve. In an example, a device operator can activate a closure mechanism by: increasing or decreasing the transmission of electricity to a solenoid; rotating a helical component; rotating a valve; moving an elastic band, ring, or loop; activating an electric detachment mechanism; changing the alignment of two openings; moving a plug; or sliding a cover.

In an example, an intrasacular aneurysm occlusion device can comprise: a proximal bowl-shaped mesh or net which is configured to span a neck of an aneurysm sac; and a distal globular mesh or net which is configured to be between the proximal bowl-shaped mesh or net and a distal dome of the aneurysm sac to hold the proximal bowl-shaped mesh or net against the neck of the aneurysm sac. In an example, the proximal bowl-shaped mesh or net can have two layers and/or double thickness. In an example, the proximal bowl-shaped mesh or net can be formed by compressing, folding, and/or inverting a spherical or ellipsoidal mesh or net. In an example, the proximal bowl-shaped mesh or net can be formed by folding and/or inverting a spherical or ellipsoidal mesh or net along its central circumference.

In an example, the distal globular mesh or net can have a spherical, oblate spheroidal, or ellipsoidal shape. In an example, the distal globular mesh or net can have a cardioid shape. In an example, the distal globular mesh or net can have a distal inversion. In an example, the proximal bowl-shaped mesh or net and the distal globular mesh or net can have a first configuration in which they do not overlap as they travel through a catheter toward an aneurysm sac and a second configuration in which they overlap after they have been deployed in the aneurysm sac. In an example, the proximal bowl-shaped mesh or net and the distal globular mesh or net can have a first configuration in which they are not nested as they travel through a catheter toward an aneurysm sac and a second configuration in which they are nested after they have been deployed in the aneurysm sac.

In an example, the proximal bowl-shaped mesh or net and the distal globular mesh or net can be moved toward each other when a user pulls, rotates, or pushes a wire or string. In an example, there can be a central opening, hole, tube, and/or lumen on the central longitudinal axis of the distal globular mesh or net through which embolic material and/or members are inserted into the distal globular mesh or net.

In an example, an aneurysm occlusion device can comprise: an intrasacular arcuate proximal stent; and an intrasacular arcuate distal stent, wherein the proximal stent has a concavity into which a portion of the distal stent fits when the device is deployed within an aneurysm sac. In an example, the distal stent can be spherical when expanded and the proximal stent can be hemispherical when expanded. In an example, the distal stent can be ellipsoidal when expanded and the proximal stent can be a section of an ellipsoid when expanded. In an example, the device can have double thickness where it covers the aneurysm neck.

In an example, the distal and proximal stents do not overlap as they travel through a catheter, but they do overlap after they are deployed in an aneurysm sac. In an example, the distal and proximal stents can have a first configuration in which they are not nested as they travel through a catheter toward an aneurysm sac and a second configuration in which they are nested after they have been deployed in the aneurysm sac. In an example, the distal stent and/or the proximal stent can be connected to a wire or string. In an example, the distal and proximal stents can be moved toward each other when a user pulls, rotates, or pushes the wire or string.

In an example, a method for forming and deploying an intrasacular aneurysm occlusion device can comprise: (a) forming two globular meshes from a tubular mesh by radially-constraining a distal end of the tubular mesh, radially-constraining a middle portion of the tubular mesh, and radially-constraining a proximal end of the tubular mesh; (b) longitudinally-stretching and radially-compressing the two globular meshes for delivery through a catheter to an aneurysm sac; (c) inserting and radially-expanding the two globular meshes within the aneurysm sac; and (d) forming a proximal bowl-shaped mesh and a distal globular mesh within the aneurysm sac by compressing and/or folding the proximal globular mesh into bowl-shaped mesh, wherein the distal globular mesh is nested within the concavity of the proximal bowl-shaped mesh.

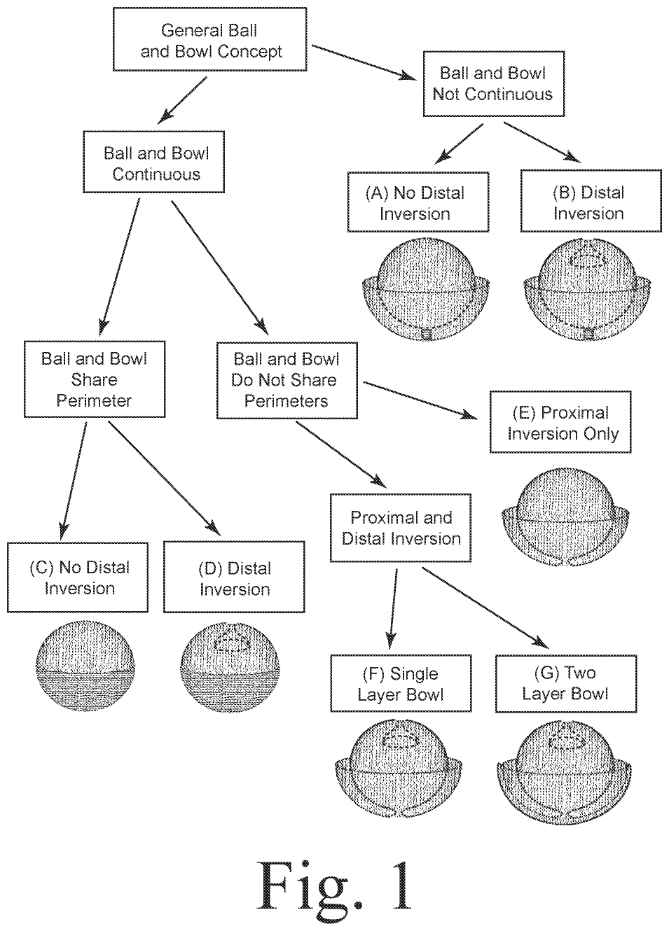

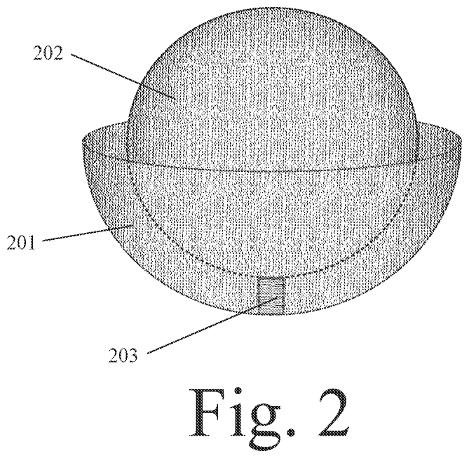

Having provided the above introduction to several embodiments of this invention, this disclosure now provides a conceptual taxonomy of general embodiments in FIG. 1 and discusses the specific embodiments shown in FIGS. 2 through 35.

FIG. 1 shows a taxonomy of general embodiments of “ball in a bowl” intrasacular aneurysm occlusion devices comprising a distal globular mesh or net (i.e. the “ball”) which is nested in the concavity of a proximal bowl-shaped mesh or net (i.e. the “bowl”). The taxonomy shown in FIG. 1 is useful as an diagrammatic introduction to some of the general ways in which this invention can be embodied. This taxonomy is structured as a decision tree, wherein each branch from a box represents a design choice for a “ball in a bowl” intrasacular aneurysm occlusion device. For each of the seven end-point designs, a generic diagram representing the design is shown. A “model” letter—(A), (B), etc. —is assigned for each of the seven end-point designs. Detailed figures with labeled components for each of these seven end-point designs will be shown in FIGS. 2 through 8.

The starting point of the device taxonomy shown in FIG. 1 is the uppermost box labeled (“General Ball and Bowl Concept”). The first branch in the taxonomy is between device designs in which the ball and bowl are continuous portions of the same structure (“Ball and Bowl Continuous”) vs. device designs in which the ball and bowl are separate structures which have been connected (“Ball and Bowl Not Continuous”). The branch with continuous ball and bowl members sub-divides between device designs in which the ball and bowl share a significant portion of their perimeters (“Ball and Bowl Share Perimeter”) vs. device designs in which the ball and bowl do not share a significant portion of their perimeters (“Ball and Bowl Do Not Share Perimeters”). The branch with shared perimeters then sub-divides between device designs in which the ball has a distal inversion (“(D) Distal Inversion”) vs. no distal inversion (“(C) No Distal Inversion”).

The branch with no significant shared perimeters sub-divides between device designs with a proximal inversion but no distal inversion (“(E) Proximal Inversion Only) vs. designs with both proximal and distal inversions. The branch with both proximal and distal inversions then sub-divides into designs wherein the bowl has a single layer (“(F) Single Layer Bowl”) vs. designs wherein the bowl has two layers (“(G) Two Layer Bowl”). Looking back up at the original branch at the top of FIG. 1, the branch in which the ball and bowl are not continuous sub-divides into designs wherein the ball has a distal inversion (“(B) Distal Inversion”) vs. no distal inversion (“(A) No Distal Inversion”).

FIG. 2 shows one embodiment of an intrasacular aneurysm occlusion device of the type which was introduced in the “(A)” branch of the device taxonomy shown in FIG. 1. This type is a “ball and bowl” device in which the ball and bowl are not continuous (e.g. they are separate components which have been connected together) and the ball has no distal inversion.

Concerning specific components, FIG. 2 shows an intrasacular aneurysm occlusion device comprising: a proximal bowl-shaped mesh or net 201 which is configured to be inserted into an aneurysm sac and expanded to span a neck of the aneurysm sac; a distal globular mesh or net 202 which is configured to be inserted into the aneurysm sac between the proximal bowl-shaped mesh and a distal dome of the aneurysm sac to hold the proximal bowl-shaped mesh against the neck of the aneurysm; and a connector 203 which connects (e.g. connects or attaches) the proximal bowl-shaped mesh or net and the distal globular mesh or net to each other.

In an example, a proximal bowl-shaped mesh or net can have a distal-facing concavity. In an example, a proximal bowl-shaped mesh or net can have a hemispherical, parabolic, or convex-lens shape. In this example, a proximal bowl-shaped mesh or net is radially symmetric, but in an alternative example it can be radially asymmetric (e.g. having radial lobes or undulations). In this example, a proximal bowl-shaped mesh or net has one layer, but in an alternative example it can have two layers. In an example, a proximal bowl-shaped mesh or net with two layers can be made by folding, inverting, or everting a mesh or net over itself. In an example, a proximal bowl-shaped mesh or net can have a resilient ring or band around its most-distal circumference, wherein this ring or band is more resilient (e.g. less flexible or lower durometers) than the rest of the mesh or net.

In this example, a distal globular mesh or net has a spherical, ball, ellipsoidal, or oblate spheroid shape, but in an alternative example, a distal globular mesh can have a generally spherical or ball shape with lobes or other irregularities to better fit an irregular-shaped aneurysm sac. In this example, a distal globular mesh or net has one layer, but in another example it could have two layers. In an example, a distal globular mesh or net can have a resilient ring or band around its (central) circumference, wherein this ring or band is more resilient (e.g. less flexible or lower durometer) than the rest of the mesh or net.

In this example, a distal globular mesh or net is (at least partially) inside a proximal bowl-shaped mesh or net. In this example, a distal globular mesh or net is (at least partially) nested within a distal-facing concavity of a proximal bowl-shaped mesh or net. In an example, a proximal bowl-shaped mesh or net can be around and outside the perimeter of part of a distal globular mesh or net. In an example, a proximal bowl-shaped mesh or net can be around and outside a proximal portion of the perimeter of part of a distal globular mesh or net. In an example, a proximal bowl-shaped mesh or net can be around the proximal half of the perimeter of part of a distal globular mesh or net. In this example, a proximal bowl-shaped mesh or net and a distal globular mesh or net are coaxial. In an example, the proximal-to-distal longitudinal axis of a proximal bowl-shaped mesh or net and a portion of the proximal-to-distal longitudinal axis of a distal globular mesh or net can overlap. In an alternative example, a proximal bowl-shaped mesh or net could be inside a distal globular mesh or net.

In this example, a proximal bowl-shaped mesh or net and a distal globular mesh or net are non-continuous (e.g. separate) components which have been connected and/or attached to each other. In this example, a proximal bowl-shaped mesh or net and a distal globular mesh or net are non-continuous components which have been created separately and then connected to each other. In an example, a proximal bowl-shaped mesh or net and a distal globular mesh or net can be connected along their central proximal-to-distal axes. In an example, embolic material can be inserted into the distal globular mesh or net through a lumen (or opening) in the inner member.

In this example, a connector connects a proximal bowl-shaped mesh or net to a distal globular mesh or net. In an example, a connector can have a circular, toroidal, or cylindrical shape. In an example, a connector can be a ring, band, tube, or threaded cylinder. In an example, a connector can comprise two coaxial members (e.g. rings and/or cylinders), an inner member and an outer member, wherein at least one of the nets or meshes is pinched (e.g. held in place by compression) between the inner member and the outer member. In an alternative example, a proximal bowl-shaped mesh or net can be attached to a distal globular mesh or net by: gluing them together with an adhesive substance; soldering, melting, or welding them together; tying them together with a wire, filament, string, or thread; riveting them together; screwing them together; or directly clipping or crimping them together.

In an alternative example, a proximal bowl-shaped mesh or net and a distal globular mesh or net can be continuous. In an alternative example, a proximal bowl-shaped mesh or net and a distal globular mesh or net can be formed from the same continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a single continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a tubular mesh or net. In an example, a proximal bowl-shaped mesh or net and a distal globular mesh or net can be made from a sequence of two globular meshes or nets, wherein the proximal one is compressed to form the proximal bowl-shaped mesh and the distal one is compressed into the concavity of the proximal bowl-shaped mesh.

In an example, a proximal bowl-shaped mesh or net can be made with different material and/or have structural attributes that are different from those of a distal globular mesh or net. In an example, a proximal bowl-shaped mesh or net can be less dense, more porous, more flexible, more elastic, and/or thinner than a distal globular mesh or net. In an example, a proximal bowl-shaped mesh or net can be made from metal, a polymer, or a combination of metal and polymer components. In an example, a proximal bowl-shaped mesh or net can be braided or woven from wires, filaments, tubes, or yarns. In an example, a proximal bowl-shaped mesh or net can be made by 3D printing. In an example, a distal globular mesh or net can be more dense, less porous, less flexible, less elastic, and/or thicker than a proximal bowl-shaped mesh or net. In an example, a distal globular mesh or net can be made from metal, a polymer, or a combination of metal and polymer components. In an example, a distal globular mesh or net can be braided or woven from wires, filaments, tubes, or yarns. In an example, a distal globular mesh or net can be made by 3D printing.

In an example, embolic members (e.g. coils, hydrogels, beads, microsponges, string-of-pearl embolics) or congealing embolic material (e.g. embolic liquid or gel) can be inserted into a distal globular mesh or net. In an example, embolic members (e.g. coils, hydrogels, beads, microsponges, string-of-pearl embolics) or congealing embolic material (e.g. embolic liquid or gel) can be inserted into a distal globular mesh or net through holes (e.g. holes, lumens, or openings) in the proximal bowl-shaped mesh or net and in the distal globular mesh or net. In an example, insertion of the embolic members and/or congealing embolic material can cause the distal globular mesh or net to (further) expand to better fit the contours of an (irregularly-shaped) aneurysm sac. In an example, embolic material can be pushed into a distal globular mesh or net using a pusher wire, a flow of liquid, a magnetic field, a rotating helical member (e.g. an Archimedes Screw), or a conveyor belt mechanism.

In an example, a connector can have a central lumen (e.g. hole, lumen, or opening) through which embolic members (e.g. coils, hydrogels, beads, microsponges, string-of-pearl embolics) or congealing embolic material (e.g. embolic liquid or gel) can be inserted into a distal globular mesh or net. In an example, a device can further comprise a closure mechanism which can close a lumen (e.g. hole, lumen, or opening) in a proximal bowl-shaped mesh or net and/or a distal globular mesh or net after embolic members and/or congealing material has been inserted through it. In an example, a closure mechanism can be remotely activated by the operator of the device. In an example, a closure mechanism can be a multi-leaflet valve, a one-way valve, a plug, an elastic band, a spring-loaded lid, a solenoid, or two or more openings which can be selectively aligned (opened) or misaligned (closed). Example variations discussed elsewhere in this disclosure or priority-linked disclosures can also be applied to this example where relevant.

FIG. 3 shows one embodiment of an intrasacular aneurysm occlusion device of the type which was introduced in the “(B)” branch of the device taxonomy which was shown in FIG. 1. This type is a “ball and bowl” device in which the ball and bowl are not continuous (e.g. they are separate components which have been connected together) and the ball has a distal inversion.

Concerning specific components, FIG. 3 shows an intrasacular aneurysm occlusion device comprising: a proximal bowl-shaped mesh or net 301 which is configured to be inserted into an aneurysm sac and expanded to span a neck of the aneurysm sac; a distal globular mesh or net 302 which is configured to be inserted into the aneurysm sac between the proximal bowl-shaped mesh and a distal dome of the aneurysm sac to hold the proximal bowl-shaped mesh against the neck of the aneurysm, wherein the distal end of the globular mesh or net has an inversion 304 (into in the interior of the globular mesh or net); and a connector 303 which connects (e.g. connects or attaches) the proximal bowl-shaped mesh or net and the distal globular mesh or net to each other.

In an example, a proximal bowl-shaped mesh or net can have a distal-facing concavity. In an example, a proximal bowl-shaped mesh or net can have a hemispherical, parabolic, or convex-lens shape. In this example, a proximal bowl-shaped mesh or net is radially symmetric, but in an alternative example it can be radially asymmetric (e.g. having radial lobes or undulations). In this example, a proximal bowl-shaped mesh or net has one layer, but in an alternative example it can have two layers. In an example, a proximal bowl-shaped mesh or net with two layers can be made by folding, inverting, or everting a mesh or net over itself. In an example, a proximal bowl-shaped mesh or net can have a resilient ring or band around its (most-distal) circumference, wherein this ring or band is more resilient (e.g. less flexible or lower durometers) than the rest of the mesh or net.

In this example, a distal globular mesh or net has a spherical, ball, ellipsoidal, or oblate spheroid shape, but in an alternative example, a distal globular mesh can have a generally spherical or ball shape with lobes or other irregularities to better fit an irregular-shaped aneurysm sac. In this example, a distal globular mesh or net has one layer, but in another example it could have two layers. In an example, a distal globular mesh or net can have a resilient ring or band around its (central) circumference, wherein this ring or band is more resilient (e.g. less flexible or lower durometer) than the rest of the mesh or net.

In this example, a distal globular mesh or net has a distal inversion. In this example, there is an inward inversion on the distal end of the globular mesh or net. In an example, this inversion can be made by inverting a distal end of a tubular mesh and radially-constraining the inverted portion with a radial band or ring. In an example, a distal inversion can extend inward between 10% and 30% of the interior diameter of a distal globular mesh or net. In an example, a distal inversion can extend inward between 20% and 55% of the interior diameter of a distal globular mesh or net. In an example, a distal inversion can extend inward between 50% and 80% of the interior diameter of a distal globular mesh or net.

In this example, a distal globular mesh or net is (at least partially) inside a proximal bowl-shaped mesh or net. In this example, a distal globular mesh or net is (at least partially) nested within a distal-facing concavity of a proximal bowl-shaped mesh or net. In an example, a proximal bowl-shaped mesh or net can be around and outside the perimeter of part of a distal globular mesh or net. In an example, a proximal bowl-shaped mesh or net can be around and outside a proximal portion of the perimeter of part of a distal globular mesh or net. In an example, a proximal bowl-shaped mesh or net can be around the proximal half of the perimeter of part of a distal globular mesh or net. In this example, a proximal bowl-shaped mesh or net and a distal globular mesh or net are coaxial. In an example, the proximal-to-distal longitudinal axis of a proximal bowl-shaped mesh or net and a portion of the proximal-to-distal longitudinal axis of a distal globular mesh or net can overlap. In an alternative example, a proximal bowl-shaped mesh or net could be inside a distal globular mesh or net.

In this example, a proximal bowl-shaped mesh or net and a distal globular mesh or net are non-continuous components which have been connected and/or attached to each other. In this example, a proximal bowl-shaped mesh or net and a distal globular mesh or net are non-continuous components which have been created separately and then connected to each other. In an example, a proximal bowl-shaped mesh or net and a distal globular mesh or net can be connected along their central proximal-to-distal axes. In an example, embolic material can be inserted into the distal globular mesh or net through a lumen (or opening) in the inner member.

In this example, a connector connects a proximal bowl-shaped mesh or net to a distal globular mesh or net. In an example, a connector can have a circular, toroidal, or cylindrical shape. In an example, a connector can be a ring, band, tube, or threaded cylinder. In an example, a connector can comprise two coaxial members (e.g. rings and/or cylinders), an inner member and an outer member, wherein at least one of the nets or meshes are pinched (e.g. held in place by compression) between the inner member and the outer member. In an alternative example, a proximal bowl-shaped mesh or net can be attached to a distal globular mesh or net by: gluing them together with an adhesive substance; soldering, melting, or welding them together; tying them together with a wire, filament, string, or thread; riveting them together; screwing them together; or directly clipping or crimping them together.

In an alternative example, a proximal bowl-shaped mesh or net and a distal globular mesh or net can be continuous. In an alternative example, a proximal bowl-shaped mesh or net and a distal globular mesh or net can be formed from the same continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a single continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a tubular mesh or net. In an example, a proximal bowl-shaped mesh or net and a distal globular mesh or net can be made from a sequence of two globular meshes or nets, wherein the proximal one is compressed to form the proximal bowl-shaped mesh and the distal one is compressed into the concavity of the proximal bowl-shaped mesh.

In an example, a proximal bowl-shaped mesh or net can be made with different material and/or have structural attributes that are different from those of a distal globular mesh or net. In an example, a proximal bowl-shaped mesh or net can be less dense, more porous, more flexible, more elastic, and/or thinner than a distal globular mesh or net. In an example, a proximal bowl-shaped mesh or net can be made from metal, a polymer, or a combination of metal and polymer components. In an example, a proximal bowl-shaped mesh or net can be braided or woven from wires, filaments, tubes, or yarns. In an example, a proximal bowl-shaped mesh or net can be made by 3D printing. In an example, a distal globular mesh or net can be more dense, less porous, less flexible, less elastic, and/or thicker than a proximal bowl-shaped mesh or net. In an example, a distal globular mesh or net can be made from metal, a polymer, or a combination of metal and polymer components. In an example, a distal globular mesh or net can be braided or woven from wires, filaments, tubes, or yarns. In an example, a distal globular mesh or net can be made by 3D printing.

In an example, embolic members (e.g. coils, hydrogels, beads, microsponges, string-of-pearl embolics) or congealing embolic material (e.g. embolic liquid or gel) can be inserted into a distal globular mesh or net. In an example, embolic members (e.g. coils, hydrogels, beads, microsponges, string-of-pearl embolics) or congealing embolic material (e.g. embolic liquid or gel) can be inserted into a distal globular mesh or net through holes (e.g. holes, lumens, or openings) in the proximal bowl-shaped mesh or net and in the distal globular mesh or net. In an example, insertion of the embolic members and/or congealing embolic material can cause the distal globular mesh or net to (further) expand to better fit the contours of an (irregularly-shaped) aneurysm sac. In an example, embolic material can be pushed into a distal globular mesh or net using a pusher wire, a flow of liquid, a magnetic field, a rotating helical member (e.g. an Archimedes Screw), or a conveyor belt mechanism.

In an example, a connector can have a central lumen (e.g. hole, lumen, or opening) through which embolic members (e.g. coils, hydrogels, beads, microsponges, string-of-pearl embolics) or congealing embolic material (e.g. embolic liquid or gel) can be inserted into a distal globular mesh or net. In an example, a device can further comprise a closure mechanism which can close a lumen (e.g. hole, lumen, or opening) in a proximal bowl-shaped mesh or net and/or a distal globular mesh or net after embolic members and/or congealing material has been inserted through it. In an example, a closure mechanism can be remotely activated by the operator of the device. In an example, a closure mechanism can be a multi-leaflet valve, a one-way valve, a plug, an elastic band, a spring-loaded lid, a solenoid, or two or more openings which can be selectively aligned (opened) or misaligned (closed). Example variations discussed elsewhere in this disclosure or priority-linked disclosures can also be applied to this example where relevant.

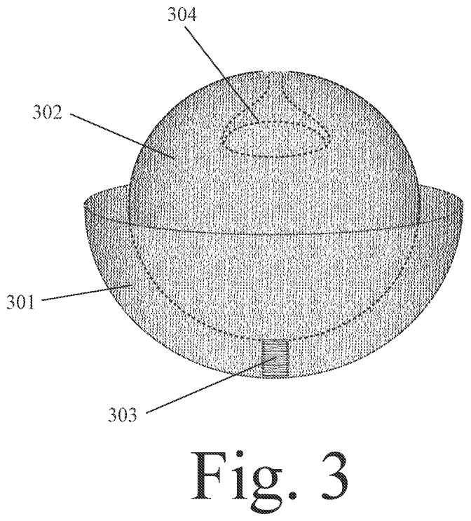

FIG. 4 shows one embodiment of an intrasacular aneurysm occlusion device of the type which was introduced in the “(C)” branch of the device taxonomy which was shown in FIG. 1. This type is a “ball and bowl” device in which the ball and bowl are continuous, the ball and bowl share a significant portion of their perimeters, and the ball has no distal inversion.

Concerning specific components, FIG. 4 shows an intrasacular aneurysm occlusion device comprising: a proximal bowl-shaped mesh or net 401 which is configured to be inserted into an aneurysm sac and expanded to span a neck of the aneurysm sac; and a distal globular mesh or net 402 which is configured to be inserted into the aneurysm sac between the proximal bowl-shaped mesh and a distal dome of the aneurysm sac to hold the proximal bowl-shaped mesh against the neck of the aneurysm.

In an example, a proximal bowl-shaped mesh or net can have a distal-facing concavity. In an example, a proximal bowl-shaped mesh or net can have a hemispherical, parabolic, or convex-lens shape. In this example, a proximal bowl-shaped mesh or net is radially symmetric, but in an alternative example it can be radially asymmetric (e.g. having radial lobes or undulations). In this example, a proximal bowl-shaped mesh or net has one layer, but in an alternative example it can have two layers. In an example, a proximal bowl-shaped mesh or net with two layers can be made by folding, inverting, or everting a mesh or net over itself. In an example, a proximal bowl-shaped mesh or net can have a resilient ring or band around its (most-distal) circumference, wherein this ring or band is more resilient (e.g. less flexible or lower durometers) than the rest of the mesh or net.

In this example, a distal globular mesh or net has a spherical, ball, ellipsoidal, or oblate spheroid shape, but in an alternative example, a distal globular mesh can have a generally spherical or ball shape with lobes or other irregularities to better fit an irregular-shaped aneurysm sac. In this example, a distal globular mesh or net has one layer, but in another example it could have two layers. In an example, a distal globular mesh or net can have a resilient ring or band around its (central) circumference, wherein this ring or band is more resilient (e.g. less flexible or lower durometer) than the rest of the mesh or net.

In this example, a distal globular mesh or net has no distal inversion. In an alternative example, there can be an inward inversion on the distal end of the globular mesh or net. In an example, an inversion can be made by inverting a distal end of a tubular mesh and then radially-constraining the inverted portion with a radial band or ring. In an example, a distal inversion can extend inward between 10% and 30% of the interior diameter of a distal globular mesh or net. In an example, a distal inversion can extend inward between 20% and 55% of the interior diameter of a distal globular mesh or net. In an example, a distal inversion can extend inward between 50% and 80% of the interior diameter of a distal globular mesh or net.

In this example, a distal globular mesh or net is (at least partially) inside a proximal bowl-shaped mesh or net. In this example, a distal globular mesh or net is (at least partially) nested within a distal-facing concavity of a proximal bowl-shaped mesh or net. In an example, a proximal bowl-shaped mesh or net can be around and outside the perimeter of part of a distal globular mesh or net. In an example, a proximal bowl-shaped mesh or net can be around and outside a proximal portion of the perimeter of part of a distal globular mesh or net. In an example, a proximal bowl-shaped mesh or net can be around the proximal half of the perimeter of part of a distal globular mesh or net. In this example, a proximal bowl-shaped mesh or net and a distal globular mesh or net are coaxial. In an example, the proximal-to-distal longitudinal axis of a proximal bowl-shaped mesh or net and a portion of the proximal-to-distal longitudinal axis of a distal globular mesh or net can overlap. In an alternative example, a proximal bowl-shaped mesh or net could be inside a distal globular mesh or net.

In this example, a proximal bowl-shaped mesh or net and a distal globular mesh or net are continuous. In this example, a proximal bowl-shaped mesh or net and a distal globular mesh or net are formed from the same continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a single continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a tubular mesh or net. In an example, a proximal bowl-shaped mesh or net and a distal globular mesh or net can be made from a sequence of two globular meshes or nets, wherein the proximal one is compressed to form the proximal bowl-shaped mesh and the distal one is compressed into the concavity of the proximal bowl-shaped mesh.

In an alternative example, a proximal bowl-shaped mesh or net and a distal globular mesh or net can be non-continuous components which have been connected and/or attached to each other. In an alternative example, a proximal bowl-shaped mesh or net and a distal globular mesh or net can be non-continuous components which have been created separately and then connected to each other. In an example, a proximal bowl-shaped mesh or net and a distal globular mesh or net can be connected along their central proximal-to-distal axes. In an example, embolic material can be inserted into the distal globular mesh or net through a lumen (or opening) in the inner member.

In an alternative example, a connector can connect a proximal bowl-shaped mesh or net to a distal globular mesh or net. In an example, a connector can have a circular, toroidal, or cylindrical shape. In an example, a connector can be a ring, band, tube, or threaded cylinder. In an example, a connector can comprise two coaxial members (e.g. rings and/or cylinders), an inner member and an outer member, wherein at least one of the nets or meshes are pinched (e.g. held in place by compression) between the inner member and the outer member. In an alternative example, a proximal bowl-shaped mesh or net can be attached to a distal globular mesh or net by: gluing them together with an adhesive substance; soldering, melting, or welding them together; tying them together with a wire, filament, string, or thread; riveting them together; screwing them together; or directly clipping or crimping them together.

In an example, a proximal bowl-shaped mesh or net can be made with different material and/or have structural attributes that are different from those of a distal globular mesh or net. In an example, a proximal bowl-shaped mesh or net can be less dense, more porous, more flexible, more elastic, and/or thinner than a distal globular mesh or net. In an example, a proximal bowl-shaped mesh or net can be made from metal, a polymer, or a combination of metal and polymer components. In an example, a proximal bowl-shaped mesh or net can be braided or woven from wires, filaments, tubes, or yarns. In an example, a proximal bowl-shaped mesh or net can be made by 3D printing. In an example, a distal globular mesh or net can be more dense, less porous, less flexible, less elastic, and/or thicker than a proximal bowl-shaped mesh or net. In an example, a distal globular mesh or net can be made from metal, a polymer, or a combination of metal and polymer components. In an example, a distal globular mesh or net can be braided or woven from wires, filaments, tubes, or yarns. In an example, a distal globular mesh or net can be made by 3D printing.

In an example, embolic members (e.g. coils, hydrogels, beads, microsponges, string-of-pearl embolics) or congealing embolic material (e.g. embolic liquid or gel) can be inserted into a distal globular mesh or net. In an example, embolic members (e.g. coils, hydrogels, beads, microsponges, string-of-pearl embolics) or congealing embolic material (e.g. embolic liquid or gel) can be inserted into a distal globular mesh or net through holes (e.g. holes, lumens, or openings) in the proximal bowl-shaped mesh or net and in the distal globular mesh or net. In an example, insertion of the embolic members and/or congealing embolic material can cause the distal globular mesh or net to (further) expand to better fit the contours of an (irregularly-shaped) aneurysm sac. In an example, embolic material can be pushed into a distal globular mesh or net using a pusher wire, a flow of liquid, a magnetic field, a rotating helical member (e.g. an Archimedes Screw), or a conveyor belt mechanism.

In an example, a device can further comprise a closure mechanism which can close a lumen (e.g. hole, lumen, or opening) in a proximal bowl-shaped mesh or net and/or a distal globular mesh or net after embolic members and/or congealing material has been inserted through it. In an example, a closure mechanism can be remotely activated by the operator of the device. In an example, a closure mechanism can be a multi-leaflet valve, a one-way valve, a plug, an elastic band, a spring-loaded lid, a solenoid, or two or more openings which can be selectively aligned (opened) or misaligned (closed). Example variations discussed elsewhere in this disclosure or priority-linked disclosures can also be applied to this example where relevant.

FIG. 5 shows one embodiment of an intrasacular aneurysm occlusion device of the type which was introduced in the “(D)” branch of the device taxonomy which was shown in FIG. 1. This type is a “ball and bowl” device in which the ball and bowl are continuous, the ball and bowl share a significant portion of their perimeters, and the ball has a distal inversion.

Concerning specific components, FIG. 5 shows an intrasacular aneurysm occlusion device comprising: a proximal bowl-shaped mesh or net 501 which is configured to be inserted into an aneurysm sac and expanded to span a neck of the aneurysm sac; and a distal globular mesh or net 502 which is configured to be inserted into the aneurysm sac between the proximal bowl-shaped mesh and a distal dome of the aneurysm sac to hold the proximal bowl-shaped mesh against the neck of the aneurysm, wherein the distal end of the globular mesh or net has an inversion 503 (into in the interior of the globular mesh or net).

In an example, a proximal bowl-shaped mesh or net can have a distal-facing concavity. In an example, a proximal bowl-shaped mesh or net can have a hemispherical, parabolic, or convex-lens shape. In this example, a proximal bowl-shaped mesh or net is radially symmetric, but in an alternative example it can be radially asymmetric (e.g. having radial lobes or undulations). In this example, a proximal bowl-shaped mesh or net has one layer, but in an alternative example it can have two layers. In an example, a proximal bowl-shaped mesh or net with two layers can be made by folding, inverting, or everting a mesh or net over itself. In an example, a proximal bowl-shaped mesh or net can have a resilient ring or band around its (most-distal) circumference, wherein this ring or band is more resilient (e.g. less flexible or lower durometers) than the rest of the mesh or net.

In this example, a distal globular mesh or net has a spherical, ball, ellipsoidal, or oblate spheroid shape, but in an alternative example, a distal globular mesh can have a generally spherical or ball shape with lobes or other irregularities to better fit an irregular-shaped aneurysm sac. In this example, a distal globular mesh or net has one layer, but in another example it could have two layers. In an example, a distal globular mesh or net can have a resilient ring or band around its (central) circumference, wherein this ring or band is more resilient (e.g. less flexible or lower durometer) than the rest of the mesh or net.

In this example, a distal globular mesh or net has a distal inversion. In this example, there is an inward inversion on the distal end of the globular mesh or net. In an example, an inversion can be made by inverting a distal end of a tubular mesh and then radially-constraining the inverted portion with a radial band or ring. In an example, a distal inversion can extend inward between 10% and 30% of the interior diameter of a distal globular mesh or net. In an example, a distal inversion can extend inward between 20% and 55% of the interior diameter of a distal globular mesh or net. In an example, a distal inversion can extend inward between 50% and 80% of the interior diameter of a distal globular mesh or net.

In this example, a distal globular mesh or net is (at least partially) inside a proximal bowl-shaped mesh or net. In this example, a distal globular mesh or net is (at least partially) nested within a distal-facing concavity of a proximal bowl-shaped mesh or net. In an example, a proximal bowl-shaped mesh or net can be around the perimeter of part of a distal globular mesh or net. In an example, a proximal bowl-shaped mesh or net can be around a proximal portion of the perimeter of part of a distal globular mesh or net. In an example, a proximal bowl-shaped mesh or net can be around the proximal half of the perimeter of part of a distal globular mesh or net. In this example, a proximal bowl-shaped mesh or net and a distal globular mesh or net are coaxial. In an example, the proximal-to-distal longitudinal axis of a proximal bowl-shaped mesh or net and a portion of the proximal-to-distal longitudinal axis of a distal globular mesh or net can overlap. In an alternative example, a proximal bowl-shaped mesh or net could be inside a distal globular mesh or net.

In this example, a proximal bowl-shaped mesh or net and a distal globular mesh or net are continuous. In this example, a proximal bowl-shaped mesh or net and a distal globular mesh or net are formed from the same continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a single continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a tubular mesh or net. In an example, a proximal bowl-shaped mesh or net and a distal globular mesh or net can be made from a sequence of two globular meshes or nets, wherein the proximal one is compressed to form the proximal bowl-shaped mesh and the distal one is compressed into the concavity of the proximal bowl-shaped mesh.

In an alternative example, a proximal bowl-shaped mesh or net and a distal globular mesh or net can be non-continuous components which have been connected and/or attached to each other. In an alternative example, a proximal bowl-shaped mesh or net and a distal globular mesh or net can be non-continuous components which have been created separately and then connected to each other. In an example, a proximal bowl-shaped mesh or net and a distal globular mesh or net can be connected along their central proximal-to-distal axes. In an example, embolic material can be inserted into the distal globular mesh or net through a lumen (or opening) in the inner member.

In an alternative example, a connector can connect a proximal bowl-shaped mesh or net to a distal globular mesh or net. In an example, a connector can have a circular, toroidal, or cylindrical shape. In an example, a connector can be a ring, band, tube, or threaded cylinder. In an example, a connector can comprise two coaxial members (e.g. rings and/or cylinders), an inner member and an outer member, wherein at least one of the nets or meshes are pinched (e.g. held in place by compression) between the inner member and the outer member. In an alternative example, a proximal bowl-shaped mesh or net can be attached to a distal globular mesh or net by: gluing them together with an adhesive substance; soldering, melting, or welding them together; tying them together with a wire, filament, string, or thread; riveting them together; screwing them together; or directly clipping or crimping them together.

In an example, a proximal bowl-shaped mesh or net can be made with different material and/or have structural attributes that are different from those of a distal globular mesh or net. In an example, a proximal bowl-shaped mesh or net can be less dense, more porous, more flexible, more elastic, and/or thinner than a distal globular mesh or net. In an example, a proximal bowl-shaped mesh or net can be made from metal, a polymer, or a combination of metal and polymer components. In an example, a proximal bowl-shaped mesh or net can be braided or woven from wires, filaments, tubes, or yarns. In an example, a proximal bowl-shaped mesh or net can be made by 3D printing. In an example, a distal globular mesh or net can be more dense, less porous, less flexible, less elastic, and/or thicker than a proximal bowl-shaped mesh or net. In an example, a distal globular mesh or net can be made from metal, a polymer, or a combination of metal and polymer components. In an example, a distal globular mesh or net can be braided or woven from wires, filaments, tubes, or yarns. In an example, a distal globular mesh or net can be made by 3D printing.

In an example, embolic members (e.g. coils, hydrogels, beads, microsponges, string-of-pearl embolics) or congealing embolic material (e.g. embolic liquid or gel) can be inserted into a distal globular mesh or net. In an example, embolic members (e.g. coils, hydrogels, beads, microsponges, string-of-pearl embolics) or congealing embolic material (e.g. embolic liquid or gel) can be inserted into a distal globular mesh or net through holes (e.g. holes, lumens, or openings) in the proximal bowl-shaped mesh or net and in the distal globular mesh or net. In an example, insertion of the embolic members and/or congealing embolic material can cause the distal globular mesh or net to (further) expand to better fit the contours of an (irregularly-shaped) aneurysm sac. In an example, embolic material can be pushed into a distal globular mesh or net using a pusher wire, a flow of liquid, a magnetic field, a rotating helical member (e.g. an Archimedes Screw), or a conveyor belt mechanism.

In an example, a device can further comprise a closure mechanism which can close a lumen (e.g. hole, lumen, or opening) in a proximal bowl-shaped mesh or net and/or a distal globular mesh or net after embolic members and/or congealing material has been inserted through it. In an example, a closure mechanism can be remotely activated by the operator of the device. In an example, a closure mechanism can be a multi-leaflet valve, a one-way valve, a plug, an elastic band, a spring-loaded lid, a solenoid, or two or more openings which can be selectively aligned (opened) or misaligned (closed). Example variations discussed elsewhere in this disclosure or priority-linked disclosures can also be applied to this example where relevant.

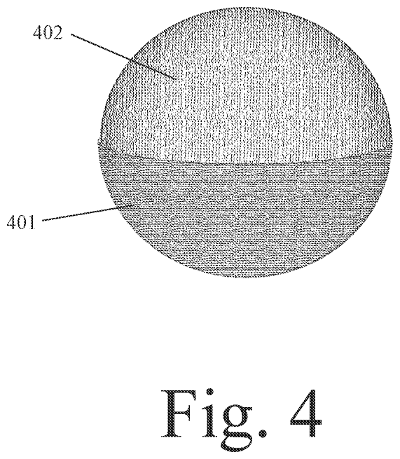

FIG. 6 shows one embodiment of an intrasacular aneurysm occlusion device of the type which was introduced in the “(E)” branch of the device taxonomy which was shown in FIG. 1. This type is a “ball and bowl” device in which the ball and bowl are continuous, the bowl has been created by a proximal inversion, and the ball has no distal inversion.

Concerning specific components, FIG. 6 shows an intrasacular aneurysm occlusion device comprising: a proximal bowl-shaped mesh or net 601 which is configured to be inserted into an aneurysm sac and expanded to span a neck of the aneurysm sac; and a distal globular mesh or net 602 which is configured to be inserted into the aneurysm sac between the proximal bowl-shaped mesh and a distal dome of the aneurysm sac to hold the proximal bowl-shaped mesh against the neck of the aneurysm; wherein the proximal bowl-shaped mesh or net and the distal globular mesh or net are made from the same continuous structure; and wherein the proximal bowl-shaped mesh or net is formed by a proximal inversion or eversion of the continuous structure.

In an example, a proximal bowl-shaped mesh or net can have a distal-facing concavity. In an example, a proximal bowl-shaped mesh or net can have a hemispherical, parabolic, or convex-lens shape. In this example, a proximal bowl-shaped mesh or net is radially symmetric, but in an alternative example it can be radially asymmetric (e.g. having radial lobes or undulations). In this example, a proximal bowl-shaped mesh or net has one layer, but in an alternative example it can have two layers. In an example, a proximal bowl-shaped mesh or net with two layers can be made by folding, inverting, or everting a mesh or net over itself. In an example, a proximal bowl-shaped mesh or net can have a resilient ring or band around its (most-distal) circumference, wherein this ring or band is more resilient (e.g. less flexible or lower durometers) than the rest of the mesh or net.

In this example, a distal globular mesh or net has a spherical, ball, ellipsoidal, or oblate spheroid shape, but in an alternative example, a distal globular mesh can have a generally spherical or ball shape with lobes or other irregularities to better fit an irregular-shaped aneurysm sac. In this example, a distal globular mesh or net has one layer, but in another example it could have two layers. In an example, a distal globular mesh or net can have a resilient ring or band around its (central) circumference, wherein this ring or band is more resilient (e.g. less flexible or lower durometer) than the rest of the mesh or net.

In this example, a distal globular mesh or net does not have a distal inversion. In an example, there can be a distal inward inversion on the distal end of the globular mesh or net. In an example, a distal inversion can be made by inverting a distal end of a tubular mesh and then radially-constraining the inverted portion with a radial band or ring. In an example, a distal inversion can extend inward between 10% and 30% of the interior diameter of a distal globular mesh or net. In an example, a distal inversion can extend inward between 20% and 55% of the interior diameter of a distal globular mesh or net. In an example, a distal inversion can extend inward between 50% and 80% of the interior diameter of a distal globular mesh or net.

In this example, a distal globular mesh or net is (at least partially) inside a proximal bowl-shaped mesh or net. In this example, a distal globular mesh or net is (at least partially) nested within a distal-facing concavity of a proximal bowl-shaped mesh or net. In an example, a proximal bowl-shaped mesh or net can be around the perimeter of part of a distal globular mesh or net. In an example, a proximal bowl-shaped mesh or net can be around a proximal portion of the perimeter of part of a distal globular mesh or net. In an example, a proximal bowl-shaped mesh or net can be around the proximal half of the perimeter of part of a distal globular mesh or net. In this example, a proximal bowl-shaped mesh or net and a distal globular mesh or net are coaxial. In an example, the proximal-to-distal longitudinal axis of a proximal bowl-shaped mesh or net and a portion of the proximal-to-distal longitudinal axis of a distal globular mesh or net can overlap. In an alternative example, a proximal bowl-shaped mesh or net could be inside a distal globular mesh or net.

In this example, a proximal bowl-shaped mesh or net and a distal globular mesh or net are continuous. In this example, a proximal bowl-shaped mesh or net and a distal globular mesh or net are formed from the same continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a single continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a tubular mesh or net. In an example, a proximal bowl-shaped mesh or net and a distal globular mesh or net can be made from a sequence of two globular meshes or nets, wherein the proximal one is compressed to form the proximal bowl-shaped mesh and the distal one is compressed into the concavity of the proximal bowl-shaped mesh.

In an example, embolic members (e.g. coils, hydrogels, beads, microsponges, string-of-pearl embolics) or congealing embolic material (e.g. embolic liquid or gel) can be inserted into a distal globular mesh or net. In an example, embolic members (e.g. coils, hydrogels, beads, microsponges, string-of-pearl embolics) or congealing embolic material (e.g. embolic liquid or gel) can be inserted into a distal globular mesh or net through holes (e.g. holes, lumens, or openings) in the proximal bowl-shaped mesh or net and in the distal globular mesh or net. In an example, insertion of the embolic members and/or congealing embolic material can cause the distal globular mesh or net to (further) expand to better fit the contours of an (irregularly-shaped) aneurysm sac. In an example, embolic material can be pushed into a distal globular mesh or net using a pusher wire, a flow of liquid, a magnetic field, a rotating helical member (e.g. an Archimedes Screw), or a conveyor belt mechanism.

In an example, a device can further comprise a closure mechanism which can close a lumen (e.g. hole, lumen, or opening) in a proximal bowl-shaped mesh or net and/or a distal globular mesh or net after embolic members and/or congealing material has been inserted through it. In an example, a closure mechanism can be remotely activated by the operator of the device. In an example, a closure mechanism can be a multi-leaflet valve, a one-way valve, a plug, an elastic band, a spring-loaded lid, a solenoid, or two or more openings which can be selectively aligned (opened) or misaligned (closed). Example variations discussed elsewhere in this disclosure or priority-linked disclosures can also be applied to this example where relevant.

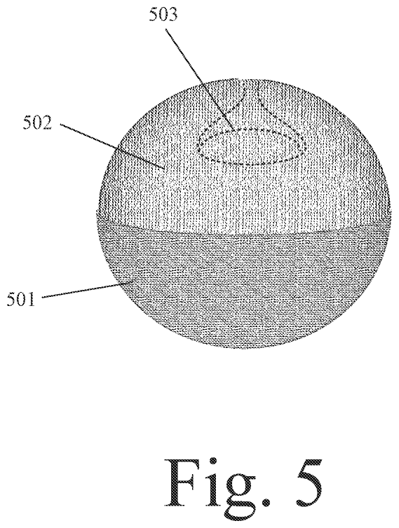

FIG. 7 shows one embodiment of an intrasacular aneurysm occlusion device of the type which was introduced in the “(F)” branch of the device taxonomy which was shown in FIG. 1. This type is a “ball and bowl” device in which the ball and bowl are continuous, the bowl has been created by a proximal inversion, and the ball has a distal inversion.

Concerning specific components, FIG. 7 shows an intrasacular aneurysm occlusion device comprising: a proximal bowl-shaped mesh or net 701 which is configured to be inserted into an aneurysm sac and expanded to span a neck of the aneurysm sac; and a distal globular mesh or net 702 which is configured to be inserted into the aneurysm sac between the proximal bowl-shaped mesh and a distal dome of the aneurysm sac to hold the proximal bowl-shaped mesh against the neck of the aneurysm, wherein the distal end of the globular mesh or net has an inversion 703 (into in the interior of the globular mesh or net); wherein the proximal bowl-shaped mesh or net and the distal globular mesh or net are made from the same continuous structure; and wherein the proximal bowl-shaped mesh or net is formed by a proximal inversion or eversion of the continuous structure.

In an example, a proximal bowl-shaped mesh or net can have a distal-facing concavity. In an example, a proximal bowl-shaped mesh or net can have a hemispherical, parabolic, or convex-lens shape. In this example, a proximal bowl-shaped mesh or net is radially symmetric, but in an alternative example it can be radially asymmetric (e.g. having radial lobes or undulations). In this example, a proximal bowl-shaped mesh or net has one layer, but in an alternative example it can have two layers. In an example, a proximal bowl-shaped mesh or net with two layers can be made by folding, inverting, or everting a mesh or net over itself. In an example, a proximal bowl-shaped mesh or net can have a resilient ring or band around its (most-distal) circumference, wherein this ring or band is more resilient (e.g. less flexible or lower durometers) than the rest of the mesh or net.

In this example, a distal globular mesh or net has a spherical, ball, ellipsoidal, or oblate spheroid shape, but in an alternative example, a distal globular mesh can have a generally spherical or ball shape with lobes or other irregularities to better fit an irregular-shaped aneurysm sac. In this example, a distal globular mesh or net has one layer, but in another example it could have two layers. In an example, a distal globular mesh or net can have a resilient ring or band around its (central) circumference, wherein this ring or band is more resilient (e.g. less flexible or lower durometer) than the rest of the mesh or net.

In this example, a distal globular mesh or net has a distal inversion. In an example, there can be a distal inward inversion on the distal end of the globular mesh or net. In an example, a distal inversion can be made by inverting a distal end of a tubular mesh and then radially-constraining the inverted portion with a radial band or ring. In an example, a distal inversion can extend inward between 10% and 30% of the interior diameter of a distal globular mesh or net. In an example, a distal inversion can extend inward between 20% and 55% of the interior diameter of a distal globular mesh or net. In an example, a distal inversion can extend inward between 50% and 80% of the interior diameter of a distal globular mesh or net.

In this example, a distal globular mesh or net is (at least partially) inside a proximal bowl-shaped mesh or net. In this example, a distal globular mesh or net is (at least partially) nested within a distal-facing concavity of a proximal bowl-shaped mesh or net. In an example, a proximal bowl-shaped mesh or net can be around the perimeter of part of a distal globular mesh or net. In an example, a proximal bowl-shaped mesh or net can be around a proximal portion of the perimeter of part of a distal globular mesh or net. In an example, a proximal bowl-shaped mesh or net can be around the proximal half of the perimeter of part of a distal globular mesh or net. In this example, a proximal bowl-shaped mesh or net and a distal globular mesh or net are coaxial. In an example, the proximal-to-distal longitudinal axis of a proximal bowl-shaped mesh or net and a portion of the proximal-to-distal longitudinal axis of a distal globular mesh or net can overlap. In an alternative example, a proximal bowl-shaped mesh or net could be inside a distal globular mesh or net.

In this example, a proximal bowl-shaped mesh or net and a distal globular mesh or net are continuous. In this example, a proximal bowl-shaped mesh or net and a distal globular mesh or net are formed from the same continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a single continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a tubular mesh or net. In an example, a proximal bowl-shaped mesh or net and a distal globular mesh or net can be made from a sequence of two globular meshes or nets, wherein the proximal one is compressed to form the proximal bowl-shaped mesh and the distal one is compressed into the concavity of the proximal bowl-shaped mesh.

In an example, embolic members (e.g. coils, hydrogels, beads, microsponges, string-of-pearl embolics) or congealing embolic material (e.g. embolic liquid or gel) can be inserted into a distal globular mesh or net. In an example, embolic members (e.g. coils, hydrogels, beads, microsponges, string-of-pearl embolics) or congealing embolic material (e.g. embolic liquid or gel) can be inserted into a distal globular mesh or net through holes (e.g. holes, lumens, or openings) in the proximal bowl-shaped mesh or net and in the distal globular mesh or net. In an example, insertion of the embolic members and/or congealing embolic material can cause the distal globular mesh or net to (further) expand to better fit the contours of an (irregularly-shaped) aneurysm sac. In an example, embolic material can be pushed into a distal globular mesh or net using a pusher wire, a flow of liquid, a magnetic field, a rotating helical member (e.g. an Archimedes Screw), or a conveyor belt mechanism.

In an example, a device can further comprise a closure mechanism which can close a lumen (e.g. hole, lumen, or opening) in a proximal bowl-shaped mesh or net and/or a distal globular mesh or net after embolic members and/or congealing material has been inserted through it. In an example, a closure mechanism can be remotely activated by the operator of the device. In an example, a closure mechanism can be a multi-leaflet valve, a one-way valve, a plug, an elastic band, a spring-loaded lid, a solenoid, or two or more openings which can be selectively aligned (opened) or misaligned (closed). Example variations discussed elsewhere in this disclosure or priority-linked disclosures can also be applied to this example where relevant.

FIG. 8 shows one embodiment of an intrasacular aneurysm occlusion device of the type which was introduced in the “(G)” branch of the device taxonomy which was shown in FIG. 1. This type is a “ball and bowl” device in which the ball and bowl are continuous, the bowl has been created by a proximal inversion, the bowl has two layers, and the ball has a distal inversion.

Concerning specific components, FIG. 8 shows an intrasacular aneurysm occlusion device comprising: a proximal bowl-shaped mesh or net further comprising two layers, 801 and 804, wherein the proximal bowl-shaped mesh or net is configured to be inserted into an aneurysm sac and expanded to span a neck of the aneurysm sac; and a distal globular mesh or net 802 which is configured to be inserted into the aneurysm sac between the proximal bowl-shaped mesh and a distal dome of the aneurysm sac to hold the proximal bowl-shaped mesh against the neck of the aneurysm, wherein the distal end of the globular mesh or net has an inversion 803 (into in the interior of the globular mesh or net); wherein the proximal bowl-shaped mesh or net and the distal globular mesh or net are made from the same continuous structure; and wherein the proximal bowl-shaped mesh or net is formed by a proximal inversion or eversion of the continuous structure.

In an example, a proximal bowl-shaped mesh or net can have a distal-facing concavity. In an example, a proximal bowl-shaped mesh or net can have a hemispherical, parabolic, or convex-lens shape. In this example, a proximal bowl-shaped mesh or net is radially symmetric, but in an alternative example it can be radially asymmetric (e.g. having radial lobes or undulations). In this example, a proximal bowl-shaped mesh or net has two layers. In an example, a proximal bowl-shaped mesh or net with two layers can be made by folding, inverting, or everting a mesh or net over itself. In an example, a proximal bowl-shaped mesh or net can have a resilient ring or band around its (most-distal) circumference, wherein this ring or band is more resilient (e.g. less flexible or lower durometers) than the rest of the mesh or net.

In this example, a distal globular mesh or net has a spherical, ball, ellipsoidal, or oblate spheroid shape, but in an alternative example, a distal globular mesh can have a generally spherical or ball shape with lobes or other irregularities to better fit an irregular-shaped aneurysm sac. In this example, a distal globular mesh or net has one layer, but in another example it could have two layers. In an example, a distal globular mesh or net can have a resilient ring or band around its (central) circumference, wherein this ring or band is more resilient (e.g. less flexible or lower durometer) than the rest of the mesh or net.

In this example, a distal globular mesh or net has a distal inversion. In an example, there can be a distal inward inversion on the distal end of the globular mesh or net. In an example, a distal inversion can be made by inverting a distal end of a tubular mesh and then radially-constraining the inverted portion with a radial band or ring. In an example, a distal inversion can extend inward between 10% and 30% of the interior diameter of a distal globular mesh or net. In an example, a distal inversion can extend inward between 20% and 55% of the interior diameter of a distal globular mesh or net. In an example, a distal inversion can extend inward between 50% and 80% of the interior diameter of a distal globular mesh or net.

In this example, a distal globular mesh or net is (at least partially) inside a proximal bowl-shaped mesh or net. In this example, a distal globular mesh or net is (at least partially) nested within a distal-facing concavity of a proximal bowl-shaped mesh or net. In an example, a proximal bowl-shaped mesh or net can be around the perimeter of part of a distal globular mesh or net. In an example, a proximal bowl-shaped mesh or net can be around a proximal portion of the perimeter of part of a distal globular mesh or net. In an example, a proximal bowl-shaped mesh or net can be around the proximal half of the perimeter of part of a distal globular mesh or net. In this example, a proximal bowl-shaped mesh or net and a distal globular mesh or net are coaxial. In an example, the proximal-to-distal longitudinal axis of a proximal bowl-shaped mesh or net and a portion of the proximal-to-distal longitudinal axis of a distal globular mesh or net can overlap. In an alternative example, a proximal bowl-shaped mesh or net could be inside a distal globular mesh or net.

In this example, a proximal bowl-shaped mesh or net and a distal globular mesh or net are continuous. In this example, a proximal bowl-shaped mesh or net and a distal globular mesh or net are formed from the same continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a single continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a tubular mesh or net. In an example, a proximal bowl-shaped mesh or net and a distal globular mesh or net can be made from a sequence of two globular meshes or nets, wherein the proximal one is compressed to form the proximal bowl-shaped mesh and the distal one is compressed into the concavity of the proximal bowl-shaped mesh.

In an example, embolic members (e.g. coils, hydrogels, beads, microsponges, string-of-pearl embolics) or congealing embolic material (e.g. embolic liquid or gel) can be inserted into a distal globular mesh or net. In an example, embolic members (e.g. coils, hydrogels, beads, microsponges, string-of-pearl embolics) or congealing embolic material (e.g. embolic liquid or gel) can be inserted into a distal globular mesh or net through holes (e.g. holes, lumens, or openings) in the proximal bowl-shaped mesh or net and in the distal globular mesh or net. In an example, insertion of the embolic members and/or congealing embolic material can cause the distal globular mesh or net to (further) expand to better fit the contours of an (irregularly-shaped) aneurysm sac. In an example, embolic material can be pushed into a distal globular mesh or net using a pusher wire, a flow of liquid, a magnetic field, a rotating helical member (e.g. an Archimedes Screw), or a conveyor belt mechanism.

In an example, a device can further comprise a closure mechanism which can close a lumen (e.g. hole, lumen, or opening) in a proximal bowl-shaped mesh or net and/or a distal globular mesh or net after embolic members and/or congealing material has been inserted through it. In an example, a closure mechanism can be remotely activated by the operator of the device. In an example, a closure mechanism can be a multi-leaflet valve, a one-way valve, a plug, an elastic band, a spring-loaded lid, a solenoid, or two or more openings which can be selectively aligned (opened) or misaligned (closed). Example variations discussed elsewhere in this disclosure or priority-linked disclosures can also be applied to this example where relevant.

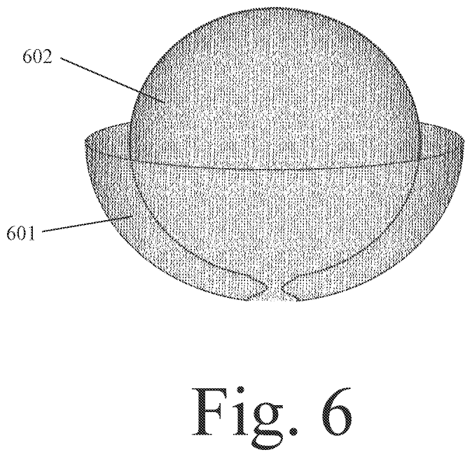

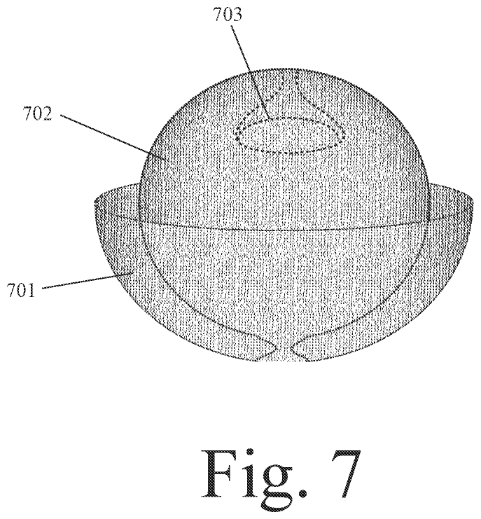

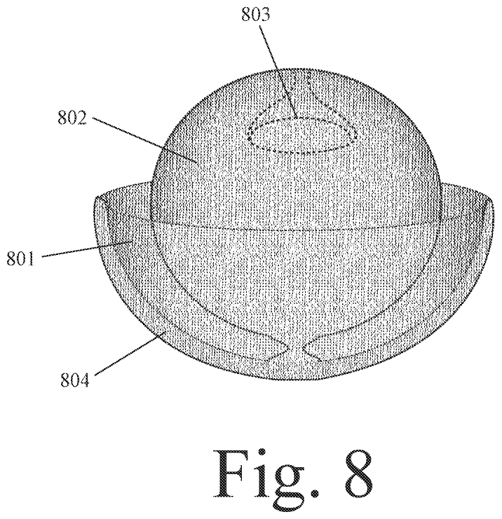

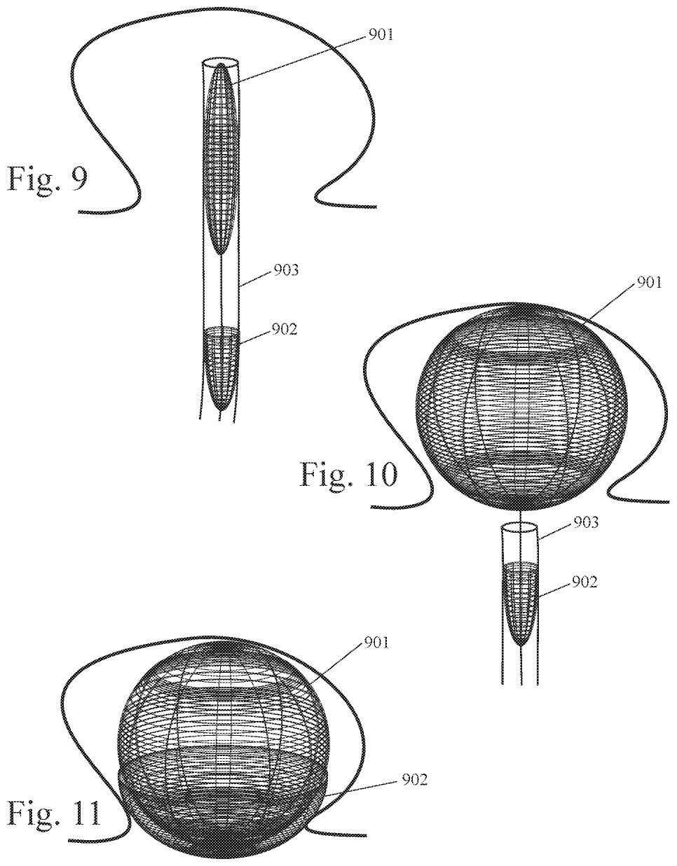

FIGS. 9 through 11 show three sequential views of an aneurysm occlusion device comprising an intrasacular arcuate distal stent 901 and an intrasacular arcuate proximal stent 902, wherein the proximal stent has a concavity into which a portion of the distal stent fits when the device is deployed within an aneurysm sac. In an example, a distal stent can be spherical when it is expanded and a proximal stent can be hemispherical when it is expanded. In an example, a proximal stent can be an inverted dome or other section of a sphere when it is expanded. In an example, an aneurysm occlusion device can comprise a distal ball stent and a proximal hemispherical stent, wherein both are expanded and overlap each other when they are deployed within an aneurysm sac. In an example, a distal surface of a proximal hemispherical stent can overlap a proximal surface of a distal ball stent. In an example, a distal stent can be an ellipsoid when it is expanded and a proximal stent can be a section of an ellipsoid when it is expanded.

In an example, a distal stent and/or a proximal stent can be a wire mesh, lattice, or net. In an example, a distal stent and/or a proximal stent can self-expand within an aneurysm sac. In an example, a distal stent and/or a proximal stent can be expanded by inflation of a balloon within it. In an example, a distal stent and a proximal stent can be inserted into an aneurysm, then expanded, and then moved toward each other so that the proximal surface of the distal stent fits into (and overlaps) the distal surface of the proximal stent. In an example, a distal stent and a proximal stent can be inserted into an aneurysm, then expanded and moved toward each other simultaneously so that the proximal stent of the distal stent fits into (and overlaps) the distal surface of the proximal stent. In an example, this can form a double-thickness wire mesh on the proximal portion of the device which covers the aneurysm neck. This can reduce blood flow into the aneurysm more completely than just a single-thickness wire mesh.

In an example, a distal stent and a proximal stent can be delivered to an aneurysm sac through a catheter 903. In an example, a distal stent can have a compressed first configuration as it is conveyed through a catheter and an expanded second configuration after it exits the catheter within an aneurysm sac. In an example, the maximum width of a distal stent in its second configuration can be wider than the aneurysm neck. In an example, a proximal stent can have a compressed first configuration as it is conveyed through a catheter and an expanded second configuration after it exits the catheter within an aneurysm sac. In an example, distal and proximal stents may not overlap in their first configurations as they travel through a catheter, but they do overlap after they are deployed in their second configurations within an aneurysm sac. In an example, distal and proximal stents can have central longitudinal axes which do not overlap in their first configurations within a catheter, but which do overlap in their second configurations within an aneurysm sac.

In an example, the distal and proximal stents which comprise this device can be coaxial. In an example, a distal stent and a proximal stent can have a first configuration in which they are not coaxial as they travel through a catheter toward an aneurysm sac and can have a second configuration in which they are coaxial after they have been deployed in the aneurysm sac. In an example, distal and proximal stents which comprise this device can be nested. In an example, a distal stent and a proximal stent can have a first configuration in which they are not nested as they travel through a catheter toward an aneurysm sac and can have a second configuration in which they are nested after they have been deployed in the aneurysm sac. In an example, distal and proximal stents which comprise this device can overlap. In an example, a distal stent and a proximal stent can have a first configuration in which they do not overlap as they travel through a catheter toward an aneurysm sac and can have a second configuration in which they do overlap after they have been deployed in the aneurysm sac.

In an example, distal and proximal stents can be connected by a wire (or string). In an example, central longitudinally axes of distal and proximal stents can be connected by a wire (or string). In an example, distal and proximal stents can be moved toward each other by a user within an aneurysm sac when the user pulls, rotates, or pushes a wire (or string) which connects the distal and proximal stents. In an example, distal and proximal stents can be moved toward each other by electromagnetism. In an example, distal and proximal stents can be simultaneously expanded and moved toward each other within an aneurysm sac. In an example, a proximal stent can cover an aneurysm neck and a distal stent can fit into a distal convex surface of the proximal stent. In an example, this can create a double-thickness wire mesh which covers (and bridges) the aneurysm neck to reduce blood flow into the aneurysm sac.

In an example, resilient wider-than-neck (e.g. proximal bowl-shaped) and flexible sac-filling (e.g. distal globular) portions of this device can be different portions of the same structure. In an example, resilient wider-than-neck (e.g. proximal bowl-shaped) and flexible sac-filling (e.g. distal globular) portions of this device can be proximal and distal portions, respectively, of the same structure. In an example, resilient wider-than-neck (e.g. proximal bowl-shaped) and flexible sac-filling (e.g. distal globular) portions of this device can be proximal and distal portions, respectively, of a single continuous structure. In an example, resilient wider-than-neck (e.g. proximal bowl-shaped) and flexible sac-filling (e.g. distal globular) portions of this device can be inner and outer portions or layers, respectively, of the same structure. In an example, resilient wider-than-neck (e.g. proximal bowl-shaped) and flexible sac-filling (e.g. distal globular) portions of this device can be inner and outer portions or layers, respectively, of the same continuous structure. In an example, resilient wider-than-neck (e.g. proximal bowl-shaped) and flexible sac-filling (e.g. distal globular) portions of this device can be different portions of the same continuous embolic structure which is inserted into an aneurysm sac.

In an example, a resilient wider-than-neck (e.g. proximal bowl-shaped) portion of this device can comprise the proximal surface of an intrasacular occlusion device and a flexible sac-filling (e.g. distal globular) portion can comprise the distal and lateral surfaces of this intrasacular occlusion device. In an example, a resilient wider-than-neck (e.g. proximal bowl-shaped) portion can be a proximal part, portion, segment, or undulation of this structure and a flexible sac-filling (e.g. distal globular) portion can be a distal (and/or peripheral) part, portion, segment, or undulation of this structure. In an example, a resilient wider-than-neck (e.g. proximal bowl-shaped) portion can be a proximal part, portion, segment, or undulation of an intrasacular aneurysm occlusion device and a flexible sac-filling (e.g. distal globular) portion can be a distal (and/or peripheral) part, portion, segment, or undulation of this device.

In an example, a resilient wider-than-neck (e.g. proximal bowl-shaped) portion and a flexible sac-filling (e.g. distal globular) portion can be different parts of the same continuous structure, with the resilient wider-than-neck (e.g. proximal bowl-shaped) portion comprising a proximal surface of the structure and the flexible sac-filling (e.g. distal globular) portion comprising a distal surface of the structure. In an example, a resilient wider-than-neck (e.g. proximal bowl-shaped) portion of this device and a flexible sac-filling (e.g. distal globular) portion of this device can be proximal and distal portions, respectively, of an intrasacular occlusion device.

FIG. 9 shows this aneurysm occlusion device at a first point in time when an arcuate distal stent and an arcuate proximal stent are in compressed configurations within a catheter as they travel toward an aneurysm sac. FIG. 10 shows this aneurysm occlusion device at a second point in time when the arcuate distal stent has exited the catheter and expanded within the aneurysm sac, but the arcuate proximal stent is still in the catheter. FIG. 11 shows this aneurysm occlusion device at a third point in time when the arcuate distal stent and the arcuate proximal stent have both been expanded and moved into overlapping positions within the aneurysm sac. They form a double-thickness wire mesh which covers the aneurysm neck. In FIG. 11, a proximal concave portion of the distal stent fits into a distal convex portion of the proximal stent. In an example, distal and proximal stents can be fused, adhered, or otherwise joined together once they are in their desired overlapping configuration.

In an example, this device can further comprise one or more additional stents which are configured to be sandwiched between the distal stent and the proximal stent when the device is deployed within an aneurysm sac. In an example, one or more additional stents can have shapes like that of the proximal stent. In an example, one or more additional stents can be sections of a sphere or ellipsoid. In an example, this can create a multiple-thickness wire mesh which covers the aneurysm neck. In an example, within pairs of contiguous stents in a longitudinal sequence of multiple stents which is deployed within an aneurysm sac, a concave portion of a relatively-distal stent can fit into (and overlap with) a convex portion of a relatively-proximal stent. In an example, a longitudinal sequence of multiple stents can be nested in each other when fully deployed in an aneurysm sac. In an example, a sequence of multiple spherical section (or ellipsoidal section) stents can fit into each other in a manner analogous to the traditional wooden doll toys called—Russian dolls. In an example, a distal portion of a device can comprise a ball stent and a proximal portion of a device can comprise an overlapping nested sequence of multiple spherical section (or ellipsoidal section) stents which covers an aneurysm neck with a multiple-thickness wire mesh. Relevant design variations discussed elsewhere in this disclosure or in priority-linked disclosures can also be applied to the example shown here.

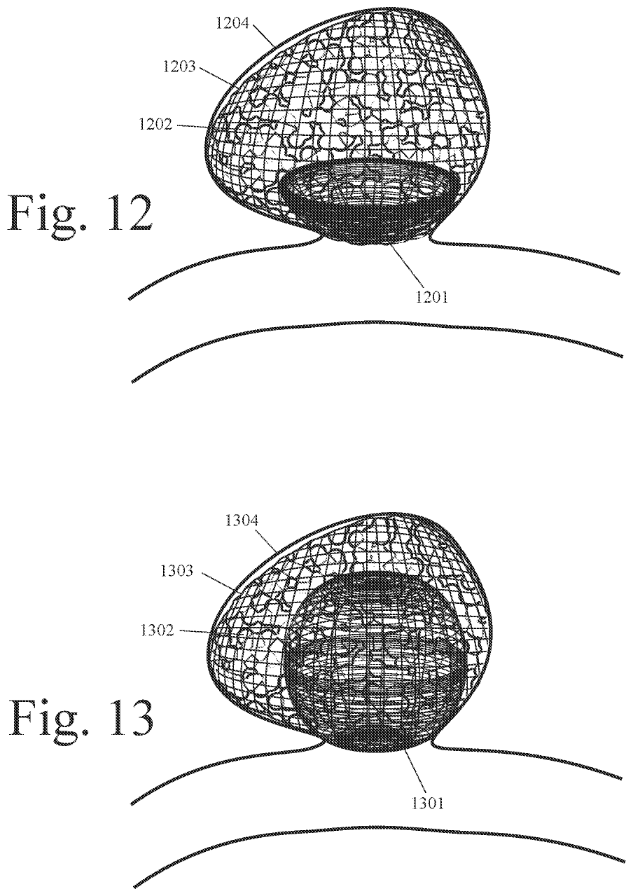

FIG. 12 shows a cross-sectional view of another example of an intrasacular aneurysm occlusion device. FIG. 12 shows a device at a single point in time after it has been fully deployed within an aneurysm sac. In this example, a resilient wider-than-neck portion of a device has a bowl shape. In an example, the resilient wider-than-neck portion of the device can self-expand into a bowl shape in a single-step transition from its first (constrained) configuration to its second (expanded) configuration. In an example, the resilient wider-than-neck portion of the device can be expanded into a bowl shape in a multi-step transition from its first (constrained) configuration to its second (expanded) configuration. In an example of a multi-step transition, the resilient wider-than-neck portion can be expanded to a spherical or ellipsoidal shape in a first step and then this sphere or ellipsoid can be collapsed into a (two-layer) bowl shape in a second step. In an example, it can be collapsed from a spherical or ellipsoidal shape to a bowl shape by pulling a wire, cord, string, or cable which is connected to its distal surface but not connected to its proximal surface.

The example shown in FIG. 12 comprises: a resilient wider-than-neck portion 1201 (such as a stent or neck bridge) of the device with a first (constrained) configuration as it is transported to an aneurysm sac 1204 and a second (expanded) configuration after it has been expanded within the aneurysm sac; wherein the resilient wider-than-neck portion in its second configuration has a width which is larger than the diameter of the neck of the aneurysm sac; and wherein the resilient wider-than-neck in its second configuration has a first level of flexibility, elasticity, and/or malleability; and a flexible sac-filling portion 1203 (such as a net or mesh) of the device with a first (constrained) configuration as it is being transported to an aneurysm sac and a second (expanded) configuration after it has been expanded within the aneurysm sac; wherein the flexible sac-filling portion is expanded from its first configuration to its second configuration by the insertion of embolic members 1202 (such as microsponges, pieces of gel, pieces of foam, beads, microspheres, or embolic coils) into the flexible sac-filling portion; and wherein the flexible sac-filling portion in its second configuration has a second level of flexibility, elasticity, and/or malleability which is greater than the first level of flexibility, elasticity, and/or malleability. Relevant embodiment variations discussed elsewhere in this disclosure or in priority-linked disclosures can also apply to this example.

FIG. 13 shows a cross-sectional view of another example of this intrasacular aneurysm occlusion device. FIG. 13 shows this device at a single point in time after it has been fully deployed within an aneurysm sac. In this example, a ball-shaped resilient wider-than-neck portion of the device is in a more proximal location. In an example, the centroid of a ball-shaped resilient wider-than-neck portion of the device can be proximal relative to the centroid of the flexible sac-filling portion of the device. In an example, the majority of the volume of the ball-shaped resilient wider-than-neck portion of this device can be configured to be located in the proximal half of the aneurysm sac. In an example, over 50% of the volume of the ball-shaped resilient wider-than-neck portion of this device can be configured to be located in the proximal half of the aneurysm sac. In an example, over 75% of the volume of the ball-shaped resilient wider-than-neck portion of this device can be configured to be located in the proximal half of the aneurysm sac.

The example shown in FIG. 13 comprises: a resilient wider-than-neck portion 1301 (such as a stent or neck bridge) of the device with a first (constrained) configuration as it is transported to an aneurysm sac 1304 and a second (expanded) configuration after it has been expanded within the aneurysm sac; wherein the resilient wider-than-neck portion in its second configuration has a width which is larger than the diameter of the neck of the aneurysm sac; and wherein the resilient wider-than-neck in its second configuration has a first level of flexibility, elasticity, and/or malleability; and a flexible sac-filling portion 1303 (such as a net or mesh) of the device with a first (constrained) configuration as it is being transported to an aneurysm sac and a second (expanded) configuration after it has been expanded within the aneurysm sac; wherein the flexible sac-filling portion is expanded from its first configuration to its second configuration by the insertion of embolic members 1302 (such as microsponges, pieces of gel, pieces of foam, beads, microspheres, or embolic coils) into the flexible sac-filling portion; and wherein the flexible sac-filling portion in its second configuration has a second level of flexibility, elasticity, and/or malleability which is greater than the first level of flexibility, elasticity, and/or malleability. Relevant embodiment variations discussed elsewhere in this disclosure or in priority-linked disclosures can also apply to this example.

FIG. 14 shows an intrasacular aneurysm occlusion device comprising a distal globular mesh or net 1401 and a proximal bowl-shaped mesh or net 1402, wherein the proximal bowl-shaped mesh or net has a concavity into which a portion of the distal globular mesh or net fits when the device is deployed within an aneurysm sac. In an example, a distal globular mesh or net can be spherical when it is expanded and a proximal bowl-shaped mesh or net can be hemispherical when it is expanded. In an example, a proximal bowl-shaped mesh or net can be an inverted dome or other section of a sphere when it is expanded. In an example, an intrasacular aneurysm occlusion device can comprise a distal ball mesh or net and a proximal hemispherical mesh or net, wherein both are expanded and overlap each other when they are deployed within an aneurysm sac. In an example, a distal surface of a proximal hemispherical mesh or net can overlap a proximal surface of a distal ball mesh or net. In an example, a distal globular mesh or net can be an ellipsoid when it is expanded and a proximal bowl-shaped mesh or net can be a section of an ellipsoid when it is expanded.

In an example, proximal bowl-shaped and distal globular portions of this device can be different portions of the same structure. In an example, proximal bowl-shaped and distal globular portions of this device can be proximal and distal portions, respectively, of the same structure. In an example, proximal bowl-shaped and distal globular portions of this device can be proximal and distal portions, respectively, of a single continuous structure. In an example, proximal bowl-shaped and distal globular portions of this device can be inner and outer portions or layers, respectively, of the same structure. In an example, proximal bowl-shaped and distal globular portions of this device can be inner and outer portions or layers, respectively, of the same continuous structure. In an example, proximal bowl-shaped and distal globular portions of this device can be different portions of the same continuous embolic structure which is inserted into an aneurysm sac.

In an example, a proximal bowl-shaped portion of this device can comprise the proximal surface of an intrasacular occlusion device and a distal globular portion can comprise the distal and lateral surfaces of this intrasacular occlusion device. In an example, a proximal bowl-shaped portion can be a proximal part, portion, segment, or undulation of this structure and a distal globular portion can be a distal (and/or peripheral) part, portion, segment, or undulation of this structure. In an example, a proximal bowl-shaped portion can be a proximal part, portion, segment, or undulation of an intrasacular aneurysm occlusion device and a distal globular portion can be a distal (and/or peripheral) part, portion, segment, or undulation of this device.

In an example, a proximal bowl-shaped portion and a distal globular portion can be different parts of the same continuous structure, with the proximal bowl-shaped portion comprising a proximal surface of the structure and the distal globular portion comprising a distal surface of the structure. In an example, a proximal bowl-shaped portion of this device and a distal globular portion of this device can be proximal and distal portions, respectively, of an intrasacular occlusion device.

In another example, distal and proximal bowl-shaped mesh or nets can be connected by a wire (or string). In an example, central longitudinally axes of distal and proximal bowl-shaped mesh or nets can be connected by a wire (or string). In an example, distal and proximal bowl-shaped mesh or nets can be moved toward each other by a user within an aneurysm sac when the user pulls, rotates, or pushes a wire (or string) which connects the distal and proximal bowl-shaped mesh or nets. In an example, distal and proximal bowl-shaped mesh or nets can be moved toward each other by electromagnetism. In an example, distal and proximal bowl-shaped mesh or nets can be simultaneously expanded and moved toward each other within an aneurysm sac. In an example, a proximal bowl-shaped mesh or net can cover an aneurysm neck and a distal globular mesh or net can fit into a distal convex surface of the proximal bowl-shaped mesh or net. In an example, this can create a double-thickness wire mesh which covers (and bridges) the aneurysm neck to reduce blood flow into the aneurysm sac.

In an example, a distal globular mesh or net and/or a proximal bowl-shaped mesh or net can be a wire mesh, lattice, or net. In an example, a distal globular mesh or net and/or a proximal bowl-shaped mesh or net can self-expand within an aneurysm sac. In an example, a distal globular mesh or net and a proximal bowl-shaped mesh or net can be inserted into an aneurysm, then expanded, and then moved toward each other so that the proximal surface of the distal globular mesh or net fits into (and overlaps) the distal surface of the proximal bowl-shaped mesh or net. In an example, a distal globular mesh or net and a proximal bowl-shaped mesh or net can be inserted into an aneurysm, then expanded and moved toward each other simultaneously so that the proximal bowl-shaped mesh or net of the distal globular mesh or net fits into (and overlaps) the distal surface of the proximal bowl-shaped mesh or net. In an example, this can form a double-thickness wire mesh on the proximal portion of the device which covers the aneurysm neck. This can reduce blood flow into the aneurysm more completely than just a single-thickness wire mesh.

In an example, a distal globular mesh or net and a proximal bowl-shaped mesh or net can be delivered to an aneurysm sac through a catheter. In an example, a distal globular mesh or net can have a compressed first configuration as it is conveyed through a catheter and an expanded second configuration after it exits the catheter within an aneurysm sac. In an example, the maximum width of a distal globular mesh or net in its second configuration can be wider than the aneurysm neck. In an example, a proximal bowl-shaped mesh or net can have a compressed first configuration as it is conveyed through a catheter and an expanded second configuration after it exits the catheter within an aneurysm sac. In an example, distal and proximal bowl-shaped mesh or nets may not overlap in their first configurations as they travel through a catheter, but they do overlap after they are deployed in their second configurations within an aneurysm sac. In an example, distal and proximal bowl-shaped mesh or nets can have central longitudinal axes which do not overlap in their first configurations within a catheter, but which do overlap in their second configurations within an aneurysm sac.

In an example, the distal and proximal bowl-shaped mesh or nets which comprise this device can be coaxial. In an example, a distal globular mesh or net and a proximal bowl-shaped mesh or net can have a first configuration in which they are not coaxial as they travel through a catheter toward an aneurysm sac and can have a second configuration in which they are coaxial after they have been deployed in the aneurysm sac. In an example, distal and proximal bowl-shaped mesh or nets which comprise this device can be nested. In an example, a distal globular mesh or net and a proximal bowl-shaped mesh or net can have a first configuration in which they are not nested as they travel through a catheter toward an aneurysm sac and can have a second configuration in which they are nested after they have been deployed in the aneurysm sac. In an example, distal and proximal bowl-shaped mesh or nets which comprise this device can overlap. In an example, a distal globular mesh or net and a proximal bowl-shaped mesh or net can have a first configuration in which they do not overlap as they travel through a catheter toward an aneurysm sac and can have a second configuration in which they do overlap after they have been deployed in the aneurysm sac. Relevant design variations discussed elsewhere in this disclosure or in priority-linked disclosures can also be applied to the example shown here.

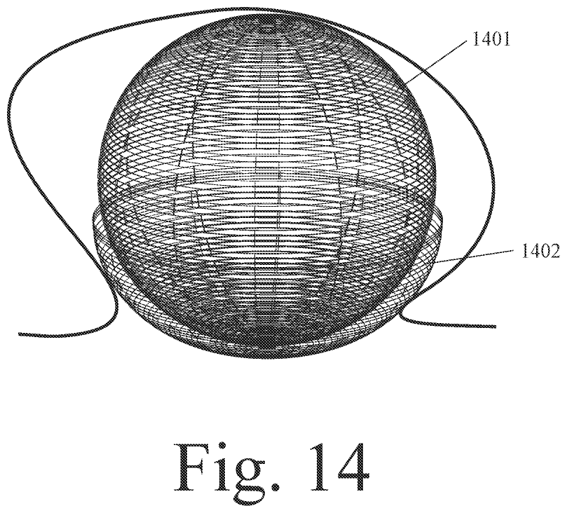

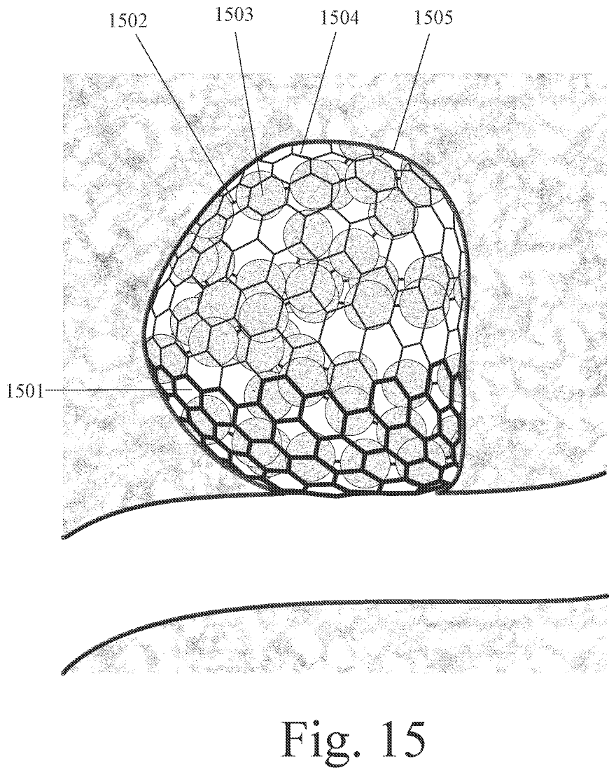

FIG. 15 shows a flexible intrasaccular aneurysm occlusion device which has been expanded by the insertion of string of pearls embolic members to conform to the irregular shape of an aneurysm sac so that blood flow through the parent vessel stops going into the aneurysm sac and the device is frictionally secured to the walls of the aneurysm sac (to reduce the possibility of protrusion into the parent vessel).

Specifically, FIG. 15 shows an embodiment of an intrasaccular aneurysm occlusion device comprising: (a) an expandable net or mesh (including portions 1501 and 1504) which is inserted into an aneurysm sac 1505; wherein a proximal portion 1501 of the expandable net or mesh is configured to be a first (average) distance from the aneurysm neck after the net or mesh has been inserted into the aneurysm sac; wherein a distal portion 1504 of the expandable net or mesh is configured to be a second (average) distance from the aneurysm neck after the net or mesh has been inserted into the aneurysm sac; wherein the second (average) distance is greater than the first (average) distance; wherein the proximal portion of the expandable net or mesh has a first level of flexibility, elasticity, conformability, and/or compliance; wherein the distal portion of the expandable net or mesh has a second level of flexibility, elasticity, conformability, and/or compliance; and wherein second level of flexibility, elasticity, conformability, and/or compliance is greater than the first level of flexibility, elasticity, conformability, and/or compliance; (b) a plurality of three-dimensional embolic members (including 1503) which are inserted into and retained within the expandable net or mesh after the net or mesh has been inserted into the aneurysm sac; and (c) one or more longitudinal strands (including 1502) which are inserted into the aneurysm sac, wherein the one or more longitudinal strands connect embolic members in the plurality of three-dimensional embolic members to each other. Embodiment variations discussed elsewhere in this disclosure and priority-linked disclosures can also be applied to this example.

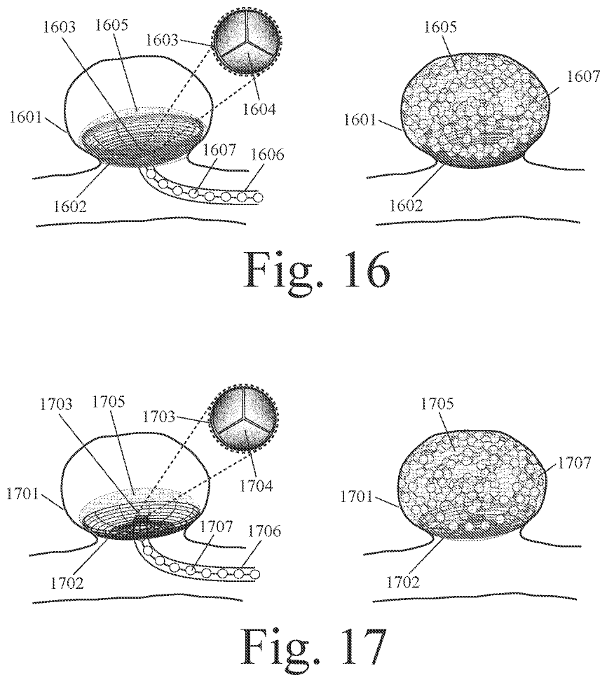

FIG. 16 shows an example of an intrasacular aneurysm occlusion device comprising a proximal bowl-shaped mesh or net and a distal globular mesh or net. The left and right sides of FIG. 16 show two sequential views of an example of an intrasacular device for occluding a cerebral aneurysm comprising: a distal globular mesh or net 1605 which is inserted into an aneurysm 1601; a proximal bowl-shaped mesh or net 1602 which is configured to be radially expanded within the aneurysm to bridge the neck of the aneurysm; a central opening 1603 in the bowl-shaped mesh or net; a valve 1604 in the central opening; and a string-of-pearls embolic member (e.g. a longitudinal series of embolic components which are connected by a flexible filament or wire) 1607 which is delivered through a catheter 1606 and inserted through the valve into the distal globular mesh or net, thereby expanding the distal globular mesh or net to fill the sac of even an irregularly-shaped aneurysm. The left side of FIG. 16 shows this device at a first point in time before the string-of-pearls embolic member has been inserted through the valve into the distal globular mesh or net. The right side of FIG. 16 shows this device at a second point in time after the string-of-pearls embolic member has been inserted through the valve into the distal globular mesh or net.

In an example, proximal bowl-shaped and distal globular portions of this device can be different portions of the same structure. In an example, proximal bowl-shaped and distal globular portions of this device can be proximal and distal portions, respectively, of the same structure. In an example, proximal bowl-shaped and distal globular portions of this device can be proximal and distal portions, respectively, of a single continuous structure. In an example, proximal bowl-shaped and distal globular portions of this device can be inner and outer portions or layers, respectively, of the same structure. In an example, proximal bowl-shaped and distal globular portions of this device can be inner and outer portions or layers, respectively, of the same continuous structure. In an example, proximal bowl-shaped and distal globular portions of this device can be different portions of the same continuous embolic structure which is inserted into an aneurysm sac.

In an example, a proximal bowl-shaped portion of this device can comprise the proximal surface of an intrasacular occlusion device and a distal globular portion can comprise the distal and lateral surfaces of this intrasacular occlusion device. In an example, a proximal bowl-shaped portion can be a proximal part, portion, segment, or undulation of this structure and a distal globular portion can be a distal (and/or peripheral) part, portion, segment, or undulation of this structure. In an example, a proximal bowl-shaped portion can be a proximal part, portion, segment, or undulation of an intrasacular aneurysm occlusion device and a distal globular portion can be a distal (and/or peripheral) part, portion, segment, or undulation of this device.

In an example, a proximal bowl-shaped portion and a distal globular portion can be different parts of the same continuous structure, with the proximal bowl-shaped portion comprising a proximal surface of the structure and the distal globular portion comprising a distal surface of the structure. In an example, a proximal bowl-shaped portion of this device and a distal globular portion of this device can be proximal and distal portions, respectively, of an intrasacular occlusion device.

In another example, the distal globular mesh or net can be attached to the bowl-shaped mesh or net. In an example, the distal globular mesh or net can be attached to the distal surface of the bowl-shaped mesh or net. In an example, the distal globular mesh or net can be attached to the outer perimeter of the bowl-shaped mesh or net. In an example, the distal globular mesh or net can be separate from the bowl-shaped mesh or net. In an example, the distal globular mesh or net can be made from a polymer and the bowl-shaped mesh or net can be made from metal.

In an example, a string-of-pearls embolic member can comprise a longitudinal series of embolic components (e.g. the “pearls”) which are connected by a flexible filament or wire (e.g. the “string”). In an example, the pearl components in a string-of-pearls embolic member can have an average size which is greater than the average size of openings in the distal globular mesh or net. In an example, the pearl components in a string-of-pearls embolic member can have an average size which is between 1 and 5 times the average size of openings in the distal globular mesh or net. In an example, the average length of filament or wire segments connecting pearl components in a string-of-pearls embolic member can be between 1 and 10 times the average size of the pearl components in the string-of-pearls embolic member. In an example, the average length of filament or wire segments connecting pearl components in a string-of-pearls embolic member can be between 1 and 10 times the average size of openings in the distal globular mesh or net. In an example, series of separate embolic members (e.g. microsponges or hydrogels) can be inserted instead of a string-of-pearls embolic member.

In an example, a bowl-shaped mesh or net can be a section of a sphere, ellipsoid, or torus. In an example, a bowl-shaped mesh or net can be hemispherical. In an example, the cross-sectional area of the central opening in the bowl-shaped mesh or net can be between 5% to 15% of the maximum cross-sectional area of the bowl-shaped mesh or net. In an example, the cross-sectional area of the central opening in the bowl-shaped mesh or net can be between 10% to 30% of the maximum cross-sectional area of the bowl-shaped mesh or net. In an example, a bowl-shaped mesh or net can be created geometrically by rotating a circle or ellipse around a vertical axis (in space) which is to the right or left of the circle or ellipse. In an example, a bowl-shaped mesh or net can radially expand within the aneurysm sac to a width which is greater than the width of the aneurysm neck.

In an example, a bowl-shaped mesh or net can have uniform porosity. In an example, a bowl-shaped mesh or net can have a uniform durometer level. In an example, a bowl-shaped mesh or net can have uniform elasticity. In an example, the outer perimeter of the bowl-shaped mesh or net can have greater porosity than the central portion of the bowl-shaped mesh or net. In an example, the outer perimeter of the bowl-shaped mesh or net can have a greater durometer level than the central portion of the bowl-shaped mesh or net. In an example, the outer perimeter of the bowl-shaped mesh or net can be more elastic than the central portion of the bowl-shaped mesh or net. In an example, the outer perimeter of the bowl-shaped mesh or net can have lower porosity than the central portion of the bowl-shaped mesh or net. In an example, the outer perimeter of the bowl-shaped mesh or net can have a lower durometer level than the central portion of the bowl-shaped mesh or net. In an example, the outer perimeter of the bowl-shaped mesh or net can be less elastic than the central portion of the bowl-shaped mesh or net.

In an example, a valve in a central opening can be a leaflet valve. In an example, a valve in a central opening can be a bi-leaflet valve or tri-leaflet valve, analogous to a heart valve. In an example, a valve can passively open when a string-of-pearls embolic member is pushed through it and passively close when the end of the embolic member passes or when a portion of the embolic member is detached and removed. In an example, such a valve allows a string-of-pearls embolic member to be inserted into the distal globular mesh or net after the bowl-shaped mesh or net has been expanded in the aneurysm, but closes to reduce blood flow into the aneurysm after the end of the embolic member has passed through the valve. In an alternative example, an active valve can be remotely opened and/or closed by the operator of the device. In an example, an active valve can be remotely opened and/or closed by an operator by the application of electromagnetic energy. In an example, an active valve can be remotely opened and/or closed by an operator by pulling a filament. In an example, an active valve can be remotely opened and/or closed by an operator by pushing, pulling, or rotating a wire. In an example, an active valve can be remotely opened and/or closed by an operator by cutting, pulling, or pushing a flap or plug. Example variations discussed elsewhere in this disclosure or priority-linked disclosures can also be applied to this example where relevant.

FIG. 17 shows an example of an intrasacular aneurysm occlusion device comprising a proximal bowl-shaped mesh or net and a distal globular mesh or net. The left and right sides of FIG. 17 show two sequential views of an example of an intrasacular device for occluding a cerebral aneurysm comprising: a distal globular mesh or net 1705 which is inserted into an aneurysm 1701; a proximal bowl-shaped (e.g. half-torus) mesh or net 1702 which is configured to be radially expanded within the aneurysm to bridge the neck of the aneurysm; a central opening 1703 in the bowl-shaped (e.g. half-torus) mesh or net; a valve 1704 in the central opening; and a string-of-pearls embolic member (e.g. a longitudinal series of embolic components which are connected by a flexible filament or wire) 1707 which is delivered through a catheter 1706 and inserted through the valve into the distal globular mesh or net, thereby expanding the distal globular mesh or net to fill the sac of even an irregularly-shaped aneurysm. The left side of FIG. 17 shows this device at a first point in time before the string-of-pearls embolic member has been inserted through the valve into the distal globular mesh or net. The right side of FIG. 17 shows this device at a second point in time after the string-of-pearls embolic member has been inserted through the valve into the distal globular mesh or net.

In an example, proximal bowl-shaped and distal globular portions of this device can be different portions of the same structure. In an example, proximal bowl-shaped and distal globular portions of this device can be proximal and distal portions, respectively, of the same structure. In an example, proximal bowl-shaped and distal globular portions of this device can be proximal and distal portions, respectively, of a single continuous structure. In an example, proximal bowl-shaped and distal globular portions of this device can be inner and outer portions or layers, respectively, of the same structure. In an example, proximal bowl-shaped and distal globular portions of this device can be inner and outer portions or layers, respectively, of the same continuous structure. In an example, proximal bowl-shaped and distal globular portions of this device can be different portions of the same continuous embolic structure which is inserted into an aneurysm sac.

In an example, a proximal bowl-shaped portion of this device can comprise the proximal surface of an intrasacular occlusion device and a distal globular portion can comprise the distal and lateral surfaces of this intrasacular occlusion device. In an example, a proximal bowl-shaped portion can be a proximal part, portion, segment, or undulation of this structure and a distal globular portion can be a distal (and/or peripheral) part, portion, segment, or undulation of this structure. In an example, a proximal bowl-shaped portion can be a proximal part, portion, segment, or undulation of an intrasacular aneurysm occlusion device and a distal globular portion can be a distal (and/or peripheral) part, portion, segment, or undulation of this device.

In an example, a proximal bowl-shaped portion and a distal globular portion can be different parts of the same continuous structure, with the proximal bowl-shaped portion comprising a proximal surface of the structure and the distal globular portion comprising a distal surface of the structure. In an example, a proximal bowl-shaped portion of this device and a distal globular portion of this device can be proximal and distal portions, respectively, of an intrasacular occlusion device.

In another example, the distal globular mesh or net can be attached to the bowl-shaped (e.g. half-torus) mesh or net. In an example, the distal globular mesh or net can be attached to the distal surface of the bowl-shaped (e.g. half-torus) mesh or net. In an example, the distal globular mesh or net can be attached to the outer perimeter of the bowl-shaped (e.g. half-torus) mesh or net. In an example, the distal globular mesh or net can be separate from the bowl-shaped (e.g. half-torus) mesh or net. In an example, the distal globular mesh or net can be made from a polymer and the bowl-shaped (e.g. half-torus) mesh or net can be made from metal.

In an example, a string-of-pearls embolic member can comprise a longitudinal series of embolic components (e.g. the “pearls”) which are connected by a flexible filament or wire (e.g. the “string”). In an example, the pearl components in a string-of-pearls embolic member can have an average size which is greater than the average size of openings in the distal globular mesh or net. In an example, the pearl components in a string-of-pearls embolic member can have an average size which is between 1 and 5 times the average size of openings in the distal globular mesh or net. In an example, the average length of filament or wire segments connecting pearl components in a string-of-pearls embolic member can be between 1 and 10 times the average size of the pearl components in the string-of-pearls embolic member. In an example, the average length of filament or wire segments connecting pearl components in a string-of-pearls embolic member can be between 1 and 10 times the average size of openings in the distal globular mesh or net. In an example, series of separate embolic members (e.g. microsponges or hydrogels) can be inserted instead of a string-of-pearls embolic member.

In an example, a bowl-shaped (e.g. half-torus) mesh or net can be the lower surface of the lower half of a torus. This is analogous to the lower surface of a half of a bagel lying flat on a surface. Following this analogy, the central opening in the bowl-shaped (e.g. half-torus) is analogous to the hole in a half bagel, although probably not as relatively large as the hole in a half bagel. In an example, the cross-sectional area of the central opening in the bowl-shaped (e.g. half-torus) mesh or net can be between 5% to 15% of the maximum cross-sectional area of the bowl-shaped (e.g. half-torus) mesh or net. In an example, the cross-sectional area of the central opening in the bowl-shaped (e.g. half-torus) mesh or net can be between 10% to 30% of the maximum cross-sectional area of the bowl-shaped (e.g. half-torus) mesh or net. In an example, a bowl-shaped (e.g. half-torus) mesh or net can be created geometrically by rotating an upward-opening arc (e.g. a section of a circle or a parabola) around a vertical axis (in space) which is to the right or left of the arc. In an example, the central portion of a bowl-shaped (e.g. half-torus) mesh or net can comprise an upward-rising cone, analogous to the cone of a volcano, with the opening being where the crater of a volcano would be. In an example, the bowl-shaped (e.g. half-torus) mesh or net can radially expand within the aneurysm sac to a width which is greater than the width of the aneurysm neck.

In an example, a bowl-shaped (e.g. half-torus) mesh or net can have uniform porosity. In an example, a bowl-shaped (e.g. half-torus) mesh or net can have a uniform durometer level. In an example, a bowl-shaped (e.g. half-torus) mesh or net can have uniform elasticity. In an example, the outer perimeter of the bowl-shaped (e.g. half-torus) mesh or net can have greater porosity than the central portion of the bowl-shaped (e.g. half-torus) mesh or net. In an example, the outer perimeter of the bowl-shaped (e.g. half-torus) mesh or net can have a greater durometer level than the central portion of the bowl-shaped (e.g. half-torus) mesh or net. In an example, the outer perimeter of the bowl-shaped (e.g. half-torus) mesh or net can be more elastic than the central portion of the bowl-shaped (e.g. half-torus) mesh or net. In an example, the outer perimeter of the bowl-shaped (e.g. half-torus) mesh or net can have lower porosity than the central portion of the bowl-shaped (e.g. half-torus) mesh or net. In an example, the outer perimeter of the bowl-shaped (e.g. half-torus) mesh or net can have a lower durometer level than the central portion of the bowl-shaped (e.g. half-torus) mesh or net. In an example, the outer perimeter of the bowl-shaped (e.g. half-torus) mesh or net can be less elastic than the central portion of the bowl-shaped (e.g. half-torus) mesh or net.

In an example, a valve in a central opening can be a leaflet valve. In an example, a valve in a central opening can be a bi-leaflet valve or tri-leaflet valve, analogous to a heart valve. In an example, a valve can passively open when a string-of-pearls embolic member is pushed through it and passively close when the end of the embolic member passes or when a portion of the embolic member is detached and removed. In an example, such a valve allows a string-of-pearls embolic member to be inserted into the distal globular mesh or net after the bowl-shaped (e.g. half-torus) mesh or net has been expanded in the aneurysm, but closes to reduce blood flow into the aneurysm after the end of the embolic member has passed through the valve. In an alternative example, an active valve can be remotely opened and/or closed by the operator of the device. In an example, an active valve can be remotely opened and/or closed by an operator by the application of electromagnetic energy. In an example, an active valve can be remotely opened and/or closed by an operator by pulling a filament. In an example, an active valve can be remotely opened and/or closed by an operator by pushing, pulling, or rotating a wire. In an example, an active valve can be remotely opened and/or closed by an operator by cutting, pulling, or pushing a flap or plug. Example variations discussed elsewhere in this disclosure or priority-linked disclosures can also be applied to this example where relevant.

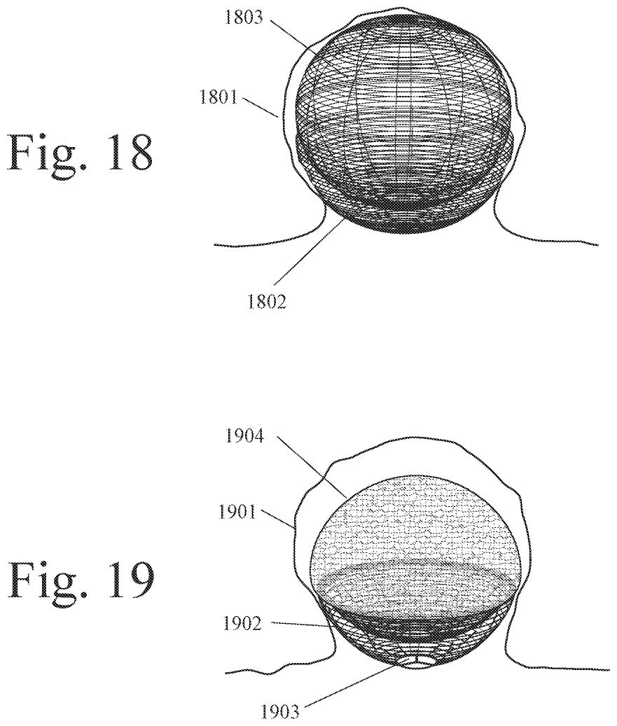

FIG. 18 shows an intrasacular device for occluding a cerebral aneurysm comprising: a distal mesh 1803 which is configured to be radially-expanded within the dome of an aneurysm 1801; and a proximal mesh 1802 which is configured to be radially-expanded to bridge the neck of the aneurysm; wherein a proximal portion of the distal mesh is nested within a concavity of the proximal mesh, wherein proximal means closer to the aneurysm neck, and wherein distal means farther from the aneurysm neck.

In an example, a proximal portion of a distal mesh can fit inside a distal concavity of a proximal mesh. In an example, a proximal portion of a distal mesh can be nested within a distal concavity of a proximal mesh. In an example, a proximal mesh can overlap a proximal portion of a distal mesh. In an example, between 20% and 40% of the surface of a distal mesh can be nested within a concavity of a proximal mesh. In an example, between 30% and 66% of the surface of a distal mesh can be nested within a concavity of a proximal mesh. In an example, distal and proximal meshes can be coaxial. In an example, the distal mesh and the proximal mesh can share a common longitudinal axis. In an example, a proximal portion of a distal mesh can be attached to a proximal mesh. In an example, a proximal portion of a distal mesh can be fused to a portion of the proximal mesh by the application of electromagnetic energy. In an example, a proximal portion of a distal mesh can be attached to a portion of a proximal mesh by a wire, string, suture, or other filament.

In an example, a distal mesh can be globular. In an example, a distal mesh can be spherical. In an example, a distal mesh can be ellipsoidal. In an example, a distal mesh can be disk shaped. In an example, a distal mesh can be toroidal. In an example, a distal mesh can be apple, barrel, or pear shaped. In an example, a distal mesh can be hyperbolic, hour-glass, dumbbell, or peanut shaped. In an example, a distal mesh can be shaped like a paper lantern. In an example, a proximal mesh can be a portion of a sphere or ellipsoid. In an example, a proximal mesh can be bowl shaped. In an example, a proximal mesh can be hemispherical. In an example, a proximal mesh can be parabolic. In an example, a proximal mesh can be a conic section.

In an example, a proximal mesh can be expanded to a diameter which is greater than the diameter of an aneurysm neck. In an example, a proximal mesh can be expanded to a diameter which is at least 90% of the maximum diameter of an aneurysm sac. In an example, a proximal mesh can be expanded to a circumference which is greater than the circumference of an aneurysm neck. In an example, a proximal mesh can be expanded to a circumference which is at least 90% of the maximum circumference of an aneurysm sac. In an example, a distal mesh can be expanded to a diameter which is between 90% and 100% of the diameter of a proximal mesh. In an example, a proximal mesh can be expanded to a circumference which is between 90% and 100% of the circumference of a proximal mesh.

In an example, a distal mesh can be a wire mesh. In an example, a distal mesh can be a wire frame. In an example, a distal mesh can be a stent. In an example, a distal mesh can be woven or braided. In an example, a distal mesh can be made from metal. In an example, a distal mesh can be made from a polymer. In an example, a distal mesh can be made from both metal and polymer components. In an example, a proximal mesh can be a wire mesh. In an example, a proximal mesh can be a wire frame. In an example, a proximal mesh can be a stent. In an example, a proximal mesh can be woven or braided. In an example, a proximal mesh can be made from metal. In an example, a proximal mesh can be made from a polymer. In an example, a proximal mesh can be made from both metal and polymer components.

In an example, proximal and distal meshes can both have the same durometer level. In an example, proximal and distal meshes can both have the same elasticity. In an example, proximal and distal meshes can both have the same porosity. In an example, a distal mesh can be less elastic than a proximal mesh. In an example, a distal mesh can have a greater durometer level than a proximal mesh. In an example, a distal mesh can have greater porosity than a proximal mesh. In an example, a distal mesh can be more elastic than a proximal mesh. In an example, a distal mesh can have a lower durometer level a proximal mesh. In an example, a distal mesh can have lower porosity than a proximal mesh.

FIG. 19 shows an intrasacular device for occluding a cerebral aneurysm comprising: a proximal bowl-shaped mesh 1902 which is configured to be radially-expanded to bridge the neck of an aneurysm 1901; a distal flexible net (or mesh) 1904 which is nested within a concavity of the bowl-shaped mesh, wherein the distal flexible net expands to fill the dome of the aneurysm; and a valve 1903 in the proximal bowl-shaped mesh through which embolic members (e.g. embolic coils, hydrogels, microsponges, beads, or string-of-pearls embolic strands) pass into the distal flexible net. In an example, the distal flexible net and the proximal bowl-shaped mesh can be connected. In an example, the distal flexible net and the proximal bowl-shaped mesh can be centrally connected. In an example, the distal flexible net and the proximal bowl-shaped mesh can both be connected to the valve.

In an example, a valve in a bowl-shaped mesh can be central to the cross-section of the bowl-shaped mesh. In an example, a valve in a bowl-shaped mesh can on the central longitudinal axis of the distal flexible net. In an example, the cross-sectional area of a valve can be between 5% to 15% of the maximum cross-sectional area of a bowl-shaped mesh. In an example, the cross-sectional area of a valve can be between 10% to 30% of the maximum cross-sectional area of a bowl-shaped mesh. In an example, a valve can be a leaflet valve. In an example, a valve can be a bi-leaflet valve or tri-leaflet valve, analogous to a heart valve.

In an example, a valve can passively open when an embolic member is pushed through it and can passively close after the member passes through or when a portion of the member is detached. In an example, such a valve allows an embolic member to be inserted into the distal flexible net, but the valve closes to reduce blood flow after the embolic member has passed through the valve. In an example, an active valve can be remotely opened and/or closed by the operator of the device. In an example, an active valve can be remotely opened and/or closed by an operator by the application of electromagnetic energy. In an example, an active valve can be remotely opened and/or closed by an operator by pulling a filament. In an example, an active valve can be remotely opened and/or closed by an operator by pushing, pulling, or rotating a wire. In an example, an active valve can be remotely opened and/or closed by an operator by cutting, pulling, or pushing a flap or plug.

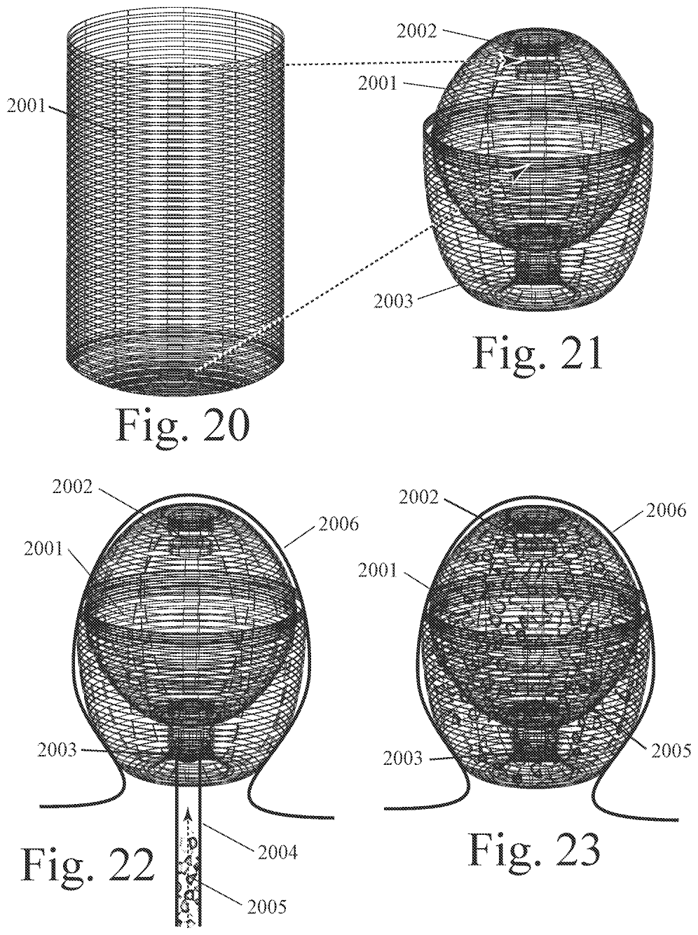

FIGS. 20 through 23 show four views, at different times, of the formation and deployment of another example of an intrasacular aneurysm occlusion device comprising: at least one annular member (in this example, mid-section annular member 2003 and distal annular member 2002), wherein an annular member is selected from the group consisting of a ring, a band, a cylinder, a tube, and a catheter; a flexible net or mesh, wherein the flexible net or mesh has a spherical, ellipsoidal, generally-globular, hemispherical, and/or bowl-shaped first configuration when it is formed by encircling, pinching, inverting, and/or everting a tubular mesh 2001 at one or more longitudinal locations using the at least one annular member; wherein the flexible net or mesh has a radially-compressed second configuration for delivery through a catheter 2004 into an aneurysm sac 2006; and wherein the flexible net or mesh is inserted and expanded within the aneurysm sac; and embolic members and/or embolic material 2005 which is inserted into the interior and/or the distal-facing concavity of the flexible net or mesh through one or more of the annular members. In this example, there are two annular members: a mid-section annular member which radially constrains a mid-section of the tubular mesh; and a distal annular member which radially constrains the distal end of the tubular mesh. In this example, the flexible net or mesh has a compound shape when it is first formed from the tubular mesh, wherein the compound shape is a globular shape inside the concavity of a bowl shape.

FIG. 20 introduces tubular mesh 2001 which is used to make the intrasacular aneurysm occlusion device. FIG. 21 shows two annular members: a mid-section annular member 2003 which radially-constrains a mid-section of the tubular mesh; and a distal annular member 2002 which radially constrains the distal end of the tubular mesh. The proximal portion of the mesh is everted over the globular distal portion of the mesh, creating a compound ball in a bowl shape as shown in FIG. 21. FIG. 22 shows this flexible mesh after it has been inserted through a catheter 2004 into an aneurysm sac 2006, wherein embolic members and/or material 2005 are starting to be delivered through the catheter (and through the annular member) into the flexible mesh and the distal portion of the aneurysm sac. FIG. 23 shows the flexible mesh and the distal portion of aneurysm sac having been filed with embolic members and/or material and the catheter having been removed.

In this example, an annular member is a ring or band which encircles a middle portion (between the ends) of the tubular mesh. In an example, an annular member can be a metal ring, band, or cylinder. In an example, an annular member can be a polymer ring, band, or cylinder. In an example, an annular member can be a wire, cord, or string. In an example, an annular member can be a ring or band which encircles a tubular mesh, thereby radially-constraining and/or pinching the tubular mesh but allowing embolic members and/or embolic material to pass through it into the interior and/or a concavity of the flexible net or mesh. In an example, an annular member can be a cylinder which encircles a tubular mesh, thereby radially-constraining and/or pinching the tubular mesh but allowing embolic members and/or embolic material to pass through it into the interior and/or a concavity of the flexible net or mesh.

In an example, an annular member can be a cord or wire which encircles a tubular mesh, thereby radially-constraining and/or pinching the tubular mesh but allowing embolic members and/or embolic material to pass through it into the interior and/or a concavity of the flexible net or mesh. In an example, an annular member can be a catheter or tube around which a tubular mesh is attached, thereby radially-constraining and/or pinching the tubular mesh but allowing embolic members and/or embolic material to pass through it into the interior and/or a concavity of the flexible net or mesh. In an example, an annular member can be a lumen through a flexible net or mesh through which embolic members and/or material is inserted into the flexible net or mesh.

In an example, a tubular mesh can be soldered, melted, glued, or crimped onto an annular member. In an example, an annular member can have an inner ring and an outer ring, wherein a tubular mesh is fixed (e.g. soldered, melted, glued, or crimped) between the two rings. In an example, an annular member can comprise an inner ring or cylinder and an outer elastic band, wherein the tubular mesh is held between the inner and outer portions. In this example, an annular member can be centrally-located with respect to a proximal surface of the flexible net or mesh. In an example, an annular member can be centrally-located with respect to the longitudinal axis of the flexible net or mesh. In an example, an annular member can be a hub into which proximal ends of braided wires or tubes of the stent are bound or attached. In an example, an annular member can be off-axial with respect to the longitudinal axis of the flexible net or mesh.

In an example, an annular member can comprise two nested and/or concentric (inner and outer) cylinders, wherein the tubular mesh is pinched and/or crimped between the two cylinders. In an example, an annular member can comprise two nested and/or concentric (inner and outer) rings or bands, wherein the tubular mesh is pinched and/or crimped between the two rings or bands. In an example, an annular member can comprise two nested and/or concentric (inner and outer) cylinders, wherein the tubular mesh is melted or glued between the two cylinders. In an example, an annular member can comprise two nested and/or concentric (inner and outer) rings or bands, wherein the tubular mesh is melted or glued between the two rings or bands.

In an example, an annular member can be a catheter which extends through the proximal surface of a flexible net or mesh, wherein the catheter is detached and/or removed after embolic members and/or material has been inserted through the catheter into the interior or distal-facing concavity of the flexible net or mesh. In an example, a distal portion of the catheter used to deliver embolic members and/or material can extend through the proximal surface of a flexible net or mesh and be detached from the rest of the catheter after embolic members and/or material has been inserted through the catheter. In an example, an annular member can be attached to a catheter during delivery of embolic members and/or material, and then detached (e.g. by the application of electromagnetic energy) from the catheter after delivery of the embolic members and/or material.

In an example, an annular member can have an outer diameter which is between 5% and 20% of the diameter of the tubular mesh before the tubular mesh is radially constrained. In an example, an annular member can have an outer diameter which is between 10% and 33% of the diameter of the tubular mesh before the tubular mesh is radially constrained. In an example, an annular member can have an outer ring (or cylinder) with a first diameter and an inner ring (or cylinder) with a second diameter, wherein the tubular mesh is crimped or pinched between the outer ring (or cylinder) and inner ring (or cylinder), and wherein the first diameter is between 50% and 75% of the second diameter. In an example, an annular member can have an outer ring (or cylinder) with a first diameter and an inner ring (or cylinder) with a second diameter, wherein the tubular mesh is crimped or pinched between the outer ring (or cylinder) and inner ring (or cylinder), and wherein the first diameter is between 66% and 90% of the second diameter.

In an example, an annular member can comprise two nested rings, bands, or cylinders, wherein a section of the tubular mesh is inserted and held between the nested rings, bands, or cylinders. In an example, an annular member can comprise an outer ring, band, or cylinder and an inner ring, band, or cylinder, wherein a section of the tubular mesh is inserted and held between them. In an example, an annular member can comprise an outer ring, band, or cylinder and an inner ring, band, or cylinder, wherein one or both of the rings, bands, or cylinders are threaded. In an example, an annular member can comprise an outer ring, band, or cylinder and an inner ring, band, or cylinder, wherein one or both of the rings, bands, or cylinders has a helical thread. In an example, an annular member can comprise an outer ring, band, or cylinder and an inner ring, band, or cylinder, wherein one or both of the rings, bands, or cylinders have a helical thread to hold a section of the tubular mesh.

In an example, this device can further comprise a closure mechanism which closes an opening through an annular member. The closure mechanism can be closed after embolic members and/or material has been inserted into a flexible net or mesh. In an example, this closure mechanism can be selected from the group consisting of: valve; electric detachment mechanism; elastic ring or band; threaded mechanism; sliding cover; sliding plug; filament loop; and electromagnetic solenoid. In an example, a closure mechanism can be a leaflet valve. In an example, a closure mechanism can be a one-way valve. In an example, a valve can allow embolic members and/or material to enter a flexible net or mesh through an opening in an annular member, but not allow the embolic members and/or material to exit the net or mesh.

In an example, a tubular mesh can be made from a polymer. In an example, a tubular mesh can be woven or braided from polymer threads, filaments, yarns, or strips. In an example, a tubular mesh can be 3D printed. In an example, a flexible net or mesh can be made from a flexible polymer. In an example, a flexible net or mesh can be made from an elastic and/or stretchable polymer. In an example, a flexible net or mesh can be elastic and/or stretchable and can expand as it is filled with embolic members and/or material. In an example, a flexible net or mesh can be sufficiently flexible to conform to the shape of even an irregularly-shaped aneurysm sac as the net or mesh is filled with embolic members and/or material. In an example, a flexible net or mesh can be sufficiently flexible to conform to the shape of even an irregularly-shaped (e.g. non-spherical) aneurysm sac as the net or mesh is filled with embolic members and/or material. In an example, a tubular mesh can be made from one or more materials selected from the group consisting of: Dacron, elastin, hydroxy-terminated polycarbonate, methylcellulose, nylon, PDMS, polybutester, polycaprolactone, polyester, polyethylene terephthalate, polypropylene, polytetrafluoroethene, polytetrafluoroethylene, polyurethane, silicone, and silk.

In an example, a tubular mesh can be made from metal. In an example, a tubular mesh can be made from Nitinol. In an example, a tubular mesh can be a flexible metal mesh. In an example, a tubular mesh can be a braided metal mesh. In an example, a tubular mesh can be woven or braided from metal filaments, wires, or tubes. In an example, a tubular mesh can be made from shape-memory material. In an example, a tubular mesh can be made with both metal and polymer components.

In an example, openings or holes in a flexible net or mesh can be smaller than the size (e.g. diameter, width, and/or length) of embolic members and/or material which is inserted into the net or mesh so that the embolic members and/or material do not escape out of the net or mesh. In an example, openings or holes in a flexible net or mesh can less than half of the size (e.g. diameter, width, and/or length) of embolic members and/or material which is inserted into the net or mesh so that the embolic members and/or material do not escape out of the net or mesh. In an example, openings or holes in a flexible net or mesh can have a size which is less than half of the smallest diameter and/or width of embolic members and/or material which is inserted into the net or mesh so that the embolic members and/or material do not escape out of the net or mesh. In an example, openings or holes in a flexible net or mesh can have a size which less than half of the smallest length of embolic members and/or material which is inserted into the net or mesh so that the embolic members and/or material do not escape out of the net or mesh.

In an example, a tubular mesh can have hexagonal openings. In an example, a tubular mesh with hexagonal openings can be made using 3D printing. In an example, a flexible metal tubular mesh with hexagonal openings can be made by 3D printing with liquid metal. In an example, a tubular mesh with hexagonal openings can be made by 3D printing with a polymer. In an example, a tubular mesh with hexagonal openings can be made by 3D printing with an elastomeric polymer. In an example, a tubular mesh with hexagonal openings can be made by 3D printing with a silicone-based polymer. In an example, a tubular mesh with hexagonal openings can be made by 3D printing with polydimethylsiloxane (PDMS).

In an example, a tubular mesh can have quadrilateral openings. In an example, a tubular mesh with quadrilateral openings can be made using 3D printing. In an example, a flexible metal tubular mesh with quadrilateral openings can be made by 3D printing with liquid metal. In an example, a tubular mesh with quadrilateral openings can be made by 3D printing with a polymer. In an example, a tubular mesh with quadrilateral openings can be made by 3D printing with an elastomeric polymer. In an example, a tubular mesh with quadrilateral openings can be made by 3D printing with a silicone-based polymer. In an example, a tubular mesh with quadrilateral openings can be made by 3D printing with polydimethylsiloxane (PDMS).

In an example, a tubular mesh can have circular openings. In an example, a tubular mesh with circular openings can be made using 3D printing. In an example, a flexible metal tubular mesh with circular openings can be made by 3D printing with liquid metal. In an example, a tubular mesh with circular openings can be made by 3D printing with a polymer. In an example, a tubular mesh with circular openings can be made by 3D printing with an elastomeric polymer. In an example, a tubular mesh with circular openings can be made by 3D printing with a silicone-based polymer. In an example, a tubular mesh with circular openings can be made by 3D printing with polydimethylsiloxane (PDMS).

In an example, a tubular mesh can be made with a cobalt chromium alloy. In an example, a tubular mesh can be made with a nickel-titanium alloy. In an example, a tubular mesh can comprise cobalt chromium alloy wires, filaments, or tubes. In an example, a tubular mesh can comprise nickel-titanium alloy wires, filaments, or tubes. In an example, a tubular mesh can comprise nitinol wires, filaments, or tubes. In an example, a tubular mesh can be made with nitinol. In an example, a tubular mesh can comprise platinum wires, filaments, or tubes. In an example, a tubular mesh can be made with platinum. In an example, a tubular mesh can comprise stainless steel wires, filaments, or tubes. In an example, a tubular mesh can be made with stainless steel. In an example, a tubular mesh can comprise tantalum wires, filaments, or tubes. In an example, a tubular mesh can be made with tantalum.

In an example, a mid-section of tubular mesh can be more flexible than the proximal and distal sections of the tubular mesh to predispose the tubular mesh to fold over itself at that mid-section to more easily form a dual-layer flexible net or mesh via inversion or eversion of the tubular mesh. In an example, a mid-section of tubular mesh can be more flexible than the proximal and distal sections of the tubular mesh to predispose the tubular mesh to fold over itself at that mid-section to more easily form a dual-layer bowl-shaped flexible net or mesh via inversion or eversion of the tubular mesh. In an example, a mid-section of tubular mesh can be less dense than the proximal and distal sections of the tubular mesh to predispose the tubular mesh to fold over itself at that mid-section to more easily form a dual-layer flexible net or mesh via inversion or eversion of the tubular mesh.

In an example, a mid-section of tubular mesh can be less dense than the proximal and distal sections of the tubular mesh to predispose the tubular mesh to fold over itself at that mid-section to more easily form a dual-layer bowl-shaped flexible net or mesh via inversion or eversion of the tubular mesh. In an example, a mid-section of tubular mesh can be made with a lower-durometer material than the proximal and distal sections of the tubular mesh to predispose the tubular mesh to fold over itself at that mid-section to more easily form a dual-layer flexible net or mesh via inversion or eversion of the tubular mesh. In an example, a mid-section of tubular mesh can be made with a lower-durometer material than the proximal and distal sections of the tubular mesh to predispose the tubular mesh to fold over itself at that mid-section to more easily form a dual-layer bowl-shaped flexible net or mesh via inversion or eversion of the tubular mesh.

In an example, a distal portion (e.g. the distal half) of a flexible net or mesh can have a lower durometer than the proximal portion (e.g. the proximal half) of the flexible net or mesh. In an example, a distal portion (e.g. the distal half) of a flexible net or mesh can be more flexible than the proximal portion (e.g. the proximal half) of the flexible net or mesh. In an example, a distal portion (e.g. the distal half) of a flexible net or mesh can be less dense than the proximal portion (e.g. the proximal half) of the flexible net or mesh. In an example, a distal portion (e.g. the distal half) of a flexible net or mesh can be more porous than the proximal portion (e.g. the proximal half) of the flexible net or mesh.

In an example, a flexible net or mesh can be folded and/or compressed as it is delivered through a catheter to an aneurysm sac. In an example, a flexible net or mesh can have radial folds as it is delivered through a catheter to an aneurysm sac. In an example, a flexible net or mesh can have longitudinal folds as it is delivered through a catheter to an aneurysm sac. In an example, a flexible net or mesh can have cross-sectional folds as it is delivered through a catheter to an aneurysm sac.

In an example, a flexible net or mesh can have a longitudinal axis which spans in a proximal-to-distal direction. Proximal can be defined as being closer to the point of entry into a person's body during delivery through the person's vasculature (in the catheter) to the aneurysm and closer to the aneurysm neck after insertion into the aneurysm sac. In this example, a tubular mesh is transformed into a ball in a bowl flexible net or mesh by: radially constraining a mid-section (or proximal one-third section) of the tubular mesh with a mid-section annular member; radially constraining the distal end of the tubular mesh with a distal annular member; and everting the proximal portion of the tubular mesh over the upper globe shape between the mid-section and the distal end.

Alternatively, a tubular mesh can be transformed into a double-layer, distally-concave, bowl-shaped flexible net or mesh by a single annular member in a middle section (between the ends) of the tubular mesh which radially-constrains the middle of the tubular mesh, wherein the proximal portion of the mesh is everted distally over the distal portion of the mesh until it has a distally-concave shape. In an example, the distal circumference of the flexible net or mesh comprises two nested tubular openings. In an example, a tubular mesh can be transformed into a single-layer, distally-concave, bowl-shaped flexible net or mesh by a single annular member which radially-constrains the proximal end of the tubular mesh. In an example, a tubular mesh can be transformed into single-layer, proximally-concave, bowl-shaped flexible net or mesh by a single annular member which radially-constrains the distal end of the tubular mesh.

In an example, a tubular mesh can be transformed into a double-layer, distally-concave, bowl-shaped flexible net or mesh by two annular members (a proximal annular member and a distal annular member) which radially-constrain the proximal and distal ends of the tubular mesh, wherein the distal portion of the tubular mesh is inverted proximally (e.g. folded proximally) until it has a distally-concave shape. In an example, the distal circumference of the flexible net or mesh can be a fold in the net or mesh. In an example, proximal and distal annular members can be aligned so that embolic members and/or material can be delivered through them into the distal-facing concavity of the double-layer bowl-shaped flexible net or mesh. In an example, both of the radially-constrained ends can project into the interior of flexible net or mesh. In an example, the proximal end can be inverted to project into the interior of bowl-shaped flexible net or mesh and the distal end is not.

In an example, a tubular mesh can be transformed into a single-layer ellipsoidal and/or generally globular flexible net or mesh by two annular members which radially-constrain the proximal and distal ends of the tubular mesh. In an example, both of these radially-constrained ends can be inverted to project into the interior of flexible net or mesh. In an example, the proximal end can be inverted to project into the interior of flexible net or mesh and the distal end can remain outside the interior of the flexible net or mesh. In an example, a tubular mesh is transformed into single-layer spherical flexible net or mesh by two annular members which radially-constrain the proximal and distal ends of the tubular mesh.

In an example, bound and/or inverted ends of a flexible net or mesh can both extend into the interior of a flexible net or mesh in a spherical, ellipsoidal, and/or globular configuration. In an example, a distal bound and/or inverted end of a flexible net or mesh can extend into the interior of a flexible net or mesh in a spherical, ellipsoidal, and/or globular configuration and a proximal bound and/or inverted end of the flexible net or mesh can extend outward from a flexible net or mesh in a spherical, ellipsoidal, and/or globular configuration. In an example, a proximal bound and/or inverted end of a flexible net or mesh can extend into the interior of a flexible net or mesh in a spherical, ellipsoidal, and/or globular configuration and a distal bound and/or inverted end of the flexible net or mesh can extend outward from a flexible net or mesh in a spherical, ellipsoidal, and/or globular configuration.

In an example, a tubular mesh can be made from polycarbonate urethane (PCU). In an example, a tubular mesh can be made from polydimethylsiloxane (PDMS). In an example, a tubular mesh can be made from polyesters. In an example, a tubular mesh can be made from polyether block amide (PEBA). In an example, a tubular mesh can be made from polyetherether ketone (PEEK). In an example, a tubular mesh can be made from polyethylene. In an example, a tubular mesh can be made from polyethylene glycol (PEG). In an example, a tubular mesh can be made from polyethylene terephthalate (PET).

In an example, a tubular mesh can be made from polyglycolic acid (PGA). In an example, a tubular mesh can be made from polylactic acid (PLA). In an example, a tubular mesh can be made from poly-N-acetylglucosamine (PNAG). In an example, a tubular mesh can be made from polyolefin. In an example, a tubular mesh can be made from polyoleandlena. In an example, a tubular mesh can be made from polypropylene. In an example, a tubular mesh can be made from polytetrafluoroethylene (PTFE). In an example, a tubular mesh can be made from polyurethane (PU). In an example, a tubular mesh can be made from polywanacrakor. In an example, a tubular mesh can be made from polyvinyl alcohol (PVA). In an example, a tubular mesh can be made from polyvinyl pyrrolidone (PVP).

In an example, a tubular mesh from which a flexible net or mesh is formed can be tapered. In an example, the distal end of a tubular mesh can have a smaller diameter than the proximal end of the tubular mesh. In an example, the distal end of a tubular mesh can have a larger diameter than the proximal end of the tubular mesh. In an example, a tubular mesh from which a flexible net or mesh is formed can have differential flexibility. In an example the distal portion of a tubular mesh can have a first level of flexibility and the proximal portion of the tubular mesh can have a second level of flexibility, wherein the first level is less than the second level. In an example the distal portion of a tubular mesh can have a first level of flexibility and the proximal portion of the tubular mesh can have a second level of flexibility, wherein the first level is greater than the second level.

In an example, a tubular mesh from which a flexible net or mesh is formed can have differential porosity. In an example the distal portion of a tubular mesh can have a first porosity level and the proximal portion of the tubular mesh can have a second porosity level, wherein the first level is less than the second level. In an example the distal portion of a tubular mesh can have a first porosity level and the proximal portion of the tubular mesh can have a second porosity level, wherein the first level is greater than the second level. In an example, a tubular mesh from which a flexible net or mesh is formed can have differential durometer. In an example the distal portion of a tubular mesh can have a first durometer level and the proximal portion of the tubular mesh can have a second durometer level, wherein the first level is less than the second level. In an example the distal portion of a tubular mesh can have a first durometer level and the proximal portion of the tubular mesh can have a second durometer level, wherein the first level is greater than the second level.

In an example, the width of a flexible net or mesh in a bowl-shaped configuration can be larger than the width of the aneurysm neck. In an example, the circumference of a flexible net or mesh in a bowl-shaped configuration can be larger than the circumference of the aneurysm neck. In an example, the width of a flexible net or mesh in a bowl-shaped configuration can be at least 10% larger than the width of the aneurysm neck. In an example, the circumference of a flexible net or mesh in a bowl-shaped configuration can be at least 10% larger than the circumference of the aneurysm neck. In an example, the width of a flexible net or mesh in a bowl-shaped configuration can be at least 90% of the maximum width of the aneurysm sac (parallel to the aneurysm neck). In an example, the circumference of a flexible net or mesh in a bowl-shaped configuration can be at least 90% of the circumference of the maximum circumference of the aneurysm sac (parallel to the aneurysm neck). In an example, a flexible net or mesh can function as a neck bridge, reducing or completely blocking blood flow from the parent vessel into the aneurysm sac.

In an example, a flexible net or mesh formed from a tubular mesh can have a generally-hemispherical shape after a tubular mesh has been radially-constrained by one or more annular members. In an example, a flexible net or mesh formed from a tubular mesh can have a generally globular and/or spherical shape after a tubular mesh has been radially-constrained by one or more annular members. In an example, a flexible net or mesh formed from a tubular mesh can have an ellipsoidal or oval shape after a tubular mesh has been radially-constrained by one or more annular members. In an example, a flexible net or mesh formed from a tubular mesh can have a disk shape after a tubular mesh has been radially-constrained by one or more annular members.

In an example, a flexible net or mesh formed from a tubular mesh can have the shape of a paraboloid-of-revolution (e.g. a paraboloid revolved around a left or right vertical edge) after a tubular mesh has been radially-constrained by one or more annular members. In an example, a flexible net or mesh formed from a tubular mesh can comprise a carlavian curve shape after a tubular mesh has been radially-constrained by one or more annular members. In an example, a flexible net or mesh formed from a tubular mesh can have a toroidal shape after a tubular mesh has been radially-constrained by one or more annular members. In an example, a flexible net or mesh formed from a tubular mesh can have a half-toroidal shape (e.g. a sliced bagel shape) after a tubular mesh has been radially-constrained by one or more annular members.

In an example, the distal end of a tubular mesh can be radially-constrained by a distal annular member and the proximal end of a tubular mesh can be radially-constrained by a proximal annular member to form a generally-globular, spherical, and/or ellipsoidal flexible net or mesh which is inserted into an aneurysm sac. In an example, the distal end of a tubular mesh can be radially-constrained by a distal annular member and the proximal end of a tubular mesh can be radially-constrained by a proximal annular member to form a generally-globular, spherical, and/or ellipsoidal shape, wherein the distal portion is then inverted and/or folded to create a two-layer bowl-shaped flexible net or mesh which is inserted into an aneurysm sac. In an example, both the distal end of a tubular mesh and the proximal end of a tubular mesh can be radially-constrained by a proximal annular member to form a two-layer bowl-shaped flexible net or mesh which is inserted into an aneurysm sac.

In an example a flexible net or mesh can be a two-layer bowl-shaped mesh with a distally-concave proximal layer and a distally-concave distal layer. In an example a flexible net or mesh can be a two-layer bowl-shaped mesh with a distally-concave proximal layer and a distally-concave distal layer, wherein the distance between the proximal and distal layers is greater in a radially-central portion of the flexible net or mesh than in radially-peripheral portions of the flexible net or mesh. In an example a flexible net or mesh can be a two-layer bowl-shaped mesh with a proximal layer and a distal layer, wherein the proximal layer has a uniform distal-facing concavity, but the distal layer has locally-concave and locally-convex portions. In an example, the radially-central portion of the distal layer is locally-convex and the radially-peripheral portions of the distal layer are locally-concave. In an example, the radially-central portion of the distal layer is less distally-concave than the radially-peripheral portions of the distal layer.

In an example, embolic members and/or material which is inserted into the flexible net or mesh can be microspheres or microballs. In an example, embolic members and/or material inserted into the flexible net or mesh can be microsponges. In an example, embolic members and/or material inserted into the flexible net or mesh can be pieces of foam. In an example, embolic members and/or material inserted into the flexible net or mesh can be microbeads. In an example, embolic members and/or material inserted into the flexible net or mesh can be pieces of hydrogel. In an example, embolic members and/or material inserted into the flexible net or mesh can be metal embolic coils. In an example, embolic members and/or material inserted into the flexible net or mesh can be embolic ribbons. In an example, embolic members and/or material inserted into the flexible net or mesh can be yarns or filaments. In an example, embolic members and/or material can be polymer strands or coils. In an example, accumulation of embolic members and/or material in an aneurysm sac can compress a flexible net or mesh from a spherical, ellipsoidal, and/or globular configuration into a hemispherical, bowl-shaped, and/or distally-concave configuration by pressing against the distal surface of the flexible net or mesh.

In an example, embolic members and/or material inserted into the flexible net or mesh can be microspheres or microballs connected by a longitudinal wire, cord, and/or filament (e.g. in a string-of-pearls configuration). In an example, embolic members and/or material inserted into the flexible net or mesh can be microsponges connected by a longitudinal wire, cord, and/or filament (e.g. in a string-of-pearls configuration). In an example, embolic members and/or material inserted into the flexible net or mesh can be pieces of foam connected by a longitudinal wire, cord, and/or filament (e.g. in a string-of-pearls configuration). In an example, embolic members and/or material inserted into the flexible net or mesh can be microbeads connected by a longitudinal wire, cord, and/or filament (e.g. in a string-of-pearls configuration).

In an example, embolic members and/or material inserted into the flexible net or mesh can be pieces of hydrogel connected by a longitudinal wire, cord, and/or filament (e.g. in a string-of-pearls configuration). In an example, embolic members and/or material inserted into the flexible net or mesh can be embolic coils connected by a longitudinal wire, cord, and/or filament (e.g. in a string-of-pearls configuration). In an example, embolic members and/or material inserted into the flexible net or mesh can be embolic ribbons connected by a longitudinal wire, cord, and/or filament (e.g. in a string-of-pearls configuration). In an example, embolic members and/or material inserted into the flexible net or mesh can be yarns or filaments connected by a longitudinal wire, cord, and/or filament (e.g. in a string-of-pearls configuration).

In an example, embolic members and/or material inserted into the flexible net or mesh can be liquid which congeals and/or solidifies. In an example, embolic members and/or material inserted into the flexible net or mesh can be a polymer which congeals and/or solidifies. In an example, embolic members and/or material inserted into the flexible net or mesh can be a liquid embolic material. In an example, embolic members and/or material inserted into the flexible net or mesh can be hydrogel material. In an example, embolic members and/or material inserted into the flexible net or mesh can be congealing adhesive material. In an example, accumulation of embolic members and/or material in an aneurysm sac can compress a flexible net or mesh from a spherical, ellipsoidal, and/or globular configuration to a hemispherical, bowl-shaped, and/or distally-concave configuration by pressing against the distal surface of the flexible net or mesh.

In an example, embolic members and/or material which is inserted through an annular member into a flexible net or mesh can be one or more mesh ribbons. In an example, embolic members and/or material which is inserted through an annular member into a flexible net or mesh can be one or more wire mesh ribbons. In an example, embolic members and/or material which is inserted through an annular member into a flexible net or mesh can be one or more polymer mesh ribbons. In an example, embolic members and/or material which is inserted through an annular member into a flexible net or mesh can be one or more undulating and/or sinusoidal ribbons. In an example, embolic members and/or material which is inserted through an annular member into a flexible net or mesh can be one or more double-layer mesh ribbons.

In an example, embolic members and/or material can be made with a cobalt chromium alloy. In an example, embolic members and/or material can be made with a nickel-titanium alloy. In an example, embolic members and/or material can be cobalt chromium alloy coils or ribbons. In an example, embolic members and/or material can be nickel-titanium alloy coils or ribbons. In an example, embolic members and/or material can be nitinol coils or ribbons. In an example, embolic members and/or material can be made with nitinol. In an example, embolic members and/or material can be platinum coils or ribbons. In an example, embolic members and/or material can be made with platinum. In an example, embolic members and/or material can be stainless steel coils or ribbons. In an example, embolic members and/or material can be made with stainless steel. In an example, embolic members and/or material can be tantalum coils or ribbons. In an example, embolic members and/or material can be made with tantalum.

In an example, embolic members and/or material can be pushed through a catheter into a flexible net or mesh by a pusher wire and/or plug. In an example, liquid embolic material (which congeals after insertion into the net or mesh) can be pushed through a catheter into a flexible net or mesh by fluid pressure. In an example, embolic members can be pushed into a flexible net or mesh by a flow of liquid (e.g. saline solution), wherein the embolic members are retained in the flexible net or mesh and the saline solution escapes out of openings in the flexible net or mesh. In an example, embolic members and/or material can be pushed through a catheter into a flexible net or mesh by a conveyer belt mechanism. In an example, embolic members and/or material can be pushed through a catheter into a flexible net or mesh by a rotating helical delivery mechanism.

In an example, embolic members which are inserted into a net or mesh can be embolic coils or ribbons. In an example, embolic members which are inserted into a net or mesh can be pieces of foam or gel (such as hydrogel). In an example, embolic members which are inserted into a net or mesh can be microballs or microspheres. In an example, embolic members which are inserted into a net or mesh can be microsponges. In an example, embolic members which are inserted into a net or mesh can be filaments or yarns. In an example, liquid embolic material can be inserted into a net or mesh.

In an example, embolic members which are inserted into a net or mesh can be selected from the group consisting of: pieces of gel; pieces of foam; and micro-sponges. In an example, embolic members which are inserted into a net or mesh can be pieces of gel, such as hydrogel. In an example, embolic members which are inserted into a net or mesh can be pieces of foam. In an example, embolic members which are inserted into a net or mesh can be micro-sponges. In an example, embolic members which are inserted into a net or mesh can be microscale gel balls. In an example, embolic members which are inserted into a net or mesh can be microscale foam balls. In an example, embolic members which are inserted into a net or mesh can be microscale sponge balls. In an example, embolic members which are inserted into a net or mesh can be microscale gel polyhedrons. In an example, embolic members which are inserted into a net or mesh can be microscale foam polyhedrons. In an example, embolic members which are inserted into a net or mesh can be microscale sponge polyhedrons.

In an example, embolic members which are inserted into a net or mesh can have generally spherical or globular shapes. In an example, embolic members which are inserted into a net or mesh can have generally prolate spherical, ellipsoidal, or ovaloid shapes. In an example, embolic members which are inserted into a net or mesh can have apple, barrel, or pair shapes. In an example, embolic members which are inserted into a net or mesh can have torus or ring shapes. In an example, embolic members which are inserted into a net or mesh can have disk or pancake shapes. In an example, embolic members which are inserted into a net or mesh can have peanut or hour-glass shapes. In an example, embolic members which are inserted into a net or mesh can be polyhedrons comprised of hexagonal surfaces. In an example, embolic members which are inserted into a net or mesh can be polyhedrons comprised of quadrilateral surfaces. In an example, embolic members which are inserted into a net or mesh can be polyhedrons comprised of triangular surfaces.

In an example, an embolic member can have a shape which is selected from the group consisting of: apple-shaped, barrel-shaped, bulbous, convex, ellipsoidal, globular, oblate spheroid, ovaloid, prolate-spheroid-shaped, spherical, and truncated-sphere-shaped. In an example, an embolic member can have a shape which is selected from the group consisting of: bowl-shaped, concave, hemispherical, and paraboloid of revolution. In an example, an embolic member can have a shape which is selected from the group consisting of: cubic, hexagon-shaped, hexahedron, octagon-shaped, octahedron, pentagonal-shaped, polyhedron-shaped, pyramidal, rectangular, square, and tetrahedronal.

In an example, embolic members which are inserted into a net or mesh can have a (diameter) size within the range of 0.5 to 2 millimeters. In an example, embolic members which are inserted into a net or mesh can have a (diameter) size within the range of 1 to 5 millimeters. In an example, embolic members which are inserted into a net or mesh can have a (diameter) size within the range of 2 to 10 millimeters. In an example, embolic members which are inserted into a net or mesh can have a (diameter) size within the range of 5 to 20 millimeters. In an example, embolic members which are inserted into a net or mesh can have a (diameter) size within the range of 0.5 to 2 microns. In an example, embolic members which are inserted into a net or mesh can have a (diameter) size within the range of 1 to 5 microns. In an example, embolic members which are inserted into a net or mesh can have a (diameter) size within the range of 2 to 10 microns. In an example, embolic members which are inserted into a net or mesh can have a (diameter) size within the range of 5 to 20 microns.

In an example, between 5 and 20 embolic members can be inserted into a net or mesh. In an example, between 10 and 50 embolic members can be inserted into a net or mesh. In an example, between 20 and 100 embolic members can be inserted into a net or mesh. In an example, between 50 and 500 embolic members can be inserted into a net or mesh.

In an example, embolic members which are inserted into a net or mesh can expand in size within the net or mesh. In an example, embolic members can have a first (average) size while being delivered to an aneurysm sac via a micro-catheter and a second (average) size after expansion within the aneurysm sac, wherein the second (average) size is 10% to 50% larger than the first (average) size. In an example, embolic members can have a first (average) size while being delivered to an aneurysm sac via a micro-catheter and a second (average) size after expansion within the aneurysm sac, wherein the second (average) size is 40% to 100% larger than the first (average) size. In an example, embolic members can have a first (average) size while being delivered to an aneurysm sac via a micro-catheter and a second (average) size after expansion within the aneurysm sac, wherein the second (average) size is more than twice the first (average) size.

In an example, embolic members can self-expand within a net or mesh after they are released from a delivery catheter. In an example, embolic members can swell upon hydration from interaction with blood or other body fluid. In an example, embolic members can be expanded within the net or mesh by one or more mechanisms selected from the group consisting of: expansion due to interaction with body fluid; expansion due to application of thermal energy; expansion due to exposure to a chemical agent; and expansion due to exposure to light energy. In an example, embolics can expand by a factor of 2-5 times. In an example, embolics can expand by a factor of 4-10 times. In an example, embolics can expand by a factor of more than 10 times. In an example, embolic members can expand to a sufficiently-large size that they cannot escape from the net or mesh after insertion into the net or mesh.

In an example, three-dimensional embolic members which are inserted into a net or mesh can be soft and compressible. In an example, three-dimensional embolic members which are inserted into a net or mesh can have a durometer less than 50. In an example, three-dimensional embolic members which are inserted into a net or mesh can have an average durometer within the range of 10 to 30. In an example, three-dimensional embolic members which are inserted into a net or mesh can have an average durometer within the range of 25 to 50. In an example, three-dimensional embolic members which are inserted into a net or mesh can have an average durometer which is less than 70.

In an example, embolic members which are inserted into a net or mesh can be made from a polymer. In an example, embolic members which are inserted into a net or mesh can be made from an elastomeric polymer. In an example, embolic members which are inserted into a net or mesh can be made from a silicone-based polymer. In an example, embolic members which are inserted into a net or mesh can be made from polydimethylsiloxane (PDMS).

In an example, an embolic member can further comprise one or more layers made with different materials. In an example, an inner layer of an embolic member can be made from a first material and an outer layer of an embolic member can be made from a second material. In an example, an inner layer of an embolic member can be made from a first material with a first durometer and an outer layer of an embolic member can be made from a second material with a second durometer, wherein the second durometer is less than the first durometer. In an example, an embolic member can have an outer layer which is adhesive. In an example, an embolic member can have an outer layer with an adhesive property which is activated by application of electromagnetic and/or thermal energy. In an example, an embolic member can have an outer layer with an adhesive property which is activated by interaction with blood.

In an example, there can be a first average durometer of embolic members which are inserted into the net or mesh at a first time and a second average durometer of embolic members which are inserted into the net or mesh at a second time, wherein the second average durometer is greater than the first average durometer. In an example, there can be a first average durometer of embolic members which are inserted into the net or mesh at a first time and a second average durometer of embolic members which are inserted into the net or mesh at a second time, wherein the second average durometer is less than the first average durometer.

In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be greater than the first average length. In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be less than the first average length.

In an example, there can be a first set of embolic members which are inserted into a net or mesh at a first time and a second set of embolic members which are inserted into the net or mesh at a second time, wherein the second set of embolic members are closer together than the first set of embolic members. In an example, there can be a first set of embolic members which are inserted into a net or mesh at a first time and a second set of embolic members which are inserted into the net or mesh at a second time, wherein the first set of embolic members are closer together than the second set of embolic members. In an example, there can be a longitudinal series of embolic members connected by one or more longitudinal strands which is inserted into a net or mesh within an aneurysm sac, wherein embolic members in the longitudinal series are progressively closer to each other moving along the length of the series in a distal to proximal manner. In an example, there can be a longitudinal series of embolic members connected by one or more longitudinal strands which is inserted into a net or mesh within an aneurysm sac, wherein embolic members in the longitudinal series are progressively farther from each other moving along the length of the series in a distal to proximal manner.

In an example, embolic members which are inserted into the net or mesh at a first time can have first shapes, embolic members which are inserted into the net or mesh at a second time can have second shapes, and the second shape can be different than the first shape. In an example, embolic members which are inserted into the net or mesh at a first time can be made with a first (combination of) material, embolic members which are inserted into the net or mesh at a second time can be made with a second (combination of) material, and the second (combination of) material can be different from the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be more flexible, elastic, and/or compliant than the first (combination of) material.

In an example, embolic members which are inserted into the net or mesh at a first time can be made with a first (combination of) material, embolic members which are inserted into the net or mesh at a second time can be made with a second (combination of) material, and the second (combination of) material can have a lower durometer than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be less flexible, elastic, and/or compliant than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can have a higher durometer than the first (combination of) material.

In an example, there can be a first average size of embolic members which are inserted into the net or mesh at a first time, a second average size of embolic members which are inserted into the net or mesh at a second time, and the second average size can be greater than the first average size. In an example, there can be a first average size of embolic members which are inserted into the net or mesh at a first time, a second average size of embolic members which are inserted into the net or mesh at a second time, and the second average size can be less than the first average size.

In an example, a net or mesh can be delivered into an aneurysm sac via a catheter and/or delivery tube. In an example, a plurality of embolic members can be delivered into the net or mesh via the same catheter and/or delivery tube. In an example, a net or mesh can be delivered into an aneurysm sac via a first catheter and/or delivery tube and a plurality of embolic members can be delivered into the net or mesh via a second catheter and/or delivery tube.

In an example, embolic members can be made from ethylene vinyl alcohol (EVA). In an example, embolic members can be made from polyolefin. In an example, embolic members can be made from fibrinogen. In an example, embolic members can be made from polylactic acid (PLA). In an example, embolic members can be made from polyethylene terephthalate (PET). In an example, embolic members can be made from steel (e.g. stainless steel). In an example, embolic members can be made from methylcellulose.

In an example, embolic members can be made from acrylic. In an example, embolic members can be made from polyethylene glycol (PEG). In an example, embolic members can be made from silk. In an example, embolic members can be made from alginate. In an example, embolic members can be made from gold. In an example, embolic members can be made from polyethylene. In an example, embolic members can be made from polyoleandlena. In an example, embolic members can be made from tantalum. In an example, embolic members can be made from cobalt-chrome alloy (cobalt chromium).

In an example, embolic members can be made from polyetherether ketone (PEEK). In an example, embolic members can be made from polywanacrakor. In an example, embolic members can be made from thermoplastic elastomer. In an example, embolic members can be made from polycarbonate urethane (PCU). In an example, embolic members can be made from water-soluble synthetic polymer. In an example, embolic members can be made from collagen. In an example, embolic members can be made from polyvinyl alcohol (PVA).

In an example, embolic members can be made from titanium. In an example, embolic members can be made from polyether block amide (PEBA). In an example, embolic members can be made from radiopaque material. In an example, embolic members can be made from copolymer. In an example, embolic members can be made from polyvinyl pyrrolidone (PVP). In an example, embolic members can be made from polydimethylsiloxane (PDMS). In an example, embolic members can be made from zirconium-based alloy. In an example, embolic members can be made from polyesters. In an example, embolic members can be made from hydrogel. In an example, embolic members can be made from silicone. In an example, embolic members can be made from nitinol (or other nickel titanium alloy).

In an example, embolic members can be made from polyglycolic acid (PGA). In an example, embolic members can be made from small intestinal submucosa. In an example, embolic members can be made from nylon. In an example, embolic members can be made from polypropylene. In an example, embolic members can be made from platinum. In an example, embolic members can be made from polyurethane (PU). In an example, embolic members can be made from tungsten. In an example, embolic members can be made from fibrin.

In an example, embolic members can be made from poly-N-acetylglucosamine (PNAG). In an example, embolic members can be made from latex. In an example, embolic members can be made from fibronectin. In an example, embolic members can be made from palladium. In an example, embolic members can be made from polytetrafluoroethylene (PTFE). In an example, embolic members can be made from gelatin.

In an example, a selected quantity, series, length, and/or volume of embolic members can be selectively dispensed and/or detached into the net or mesh in situ by a mechanism selected from the group consisting of: breaking a connection between embolic members in a series of embolic members; cutting a connection between embolic members in a series of embolic members (e.g. with a cutting edge or laser); dissolving a connection between embolic members in a series of embolic members (e.g. with thermal energy or a chemical); electrolytic mechanism; hydraulic mechanism; injecting a flow of embolic members suspended in a liquid or gel into a net or mesh; melting a connection between embolic members in a series of embolic members (e.g. with thermal or light energy); progressing embolic members into a net or mesh via a conveyor belt (e.g. chain-based conveyor); progressing embolic members into a net or mesh via a helical conveyor (e.g. with an Archimedes' screw); pushing embolic members into a net or mesh using the force of a liquid flow; pusher rod and/or plunger; release detachment mechanism; and thermal detachment mechanism.

In an example, embolic members can differ among themselves with respect to one or more characteristics selected from the group consisting of: porosity, shape, size, material, composition, coating, radiopacity, strength, stiffness, and type. In an example, a plurality of embolic members can be delivered into a net or mesh in a linear (longitudinal) array or series of inter-connected embolic members. In an example, a plurality of embolic members can be delivered into a net or mesh in a linear (longitudinal) array of connected embolic members, wherein this linear array can be cut, separated, and/or detached in situ (in a remote manner) at one or more selected locations by the user of the device in order to deliver a selected quantity, length, or volume or embolic members. In an example, a plurality of embolic members can be delivered into a net or mesh in a planar array of inter-connected embolic members. In an example, a plurality of embolic members can be delivered into a net or mesh in a three-dimensional array of inter-connected embolic members.

In an example, a series of embolic members can be delivered into a net or mesh, wherein successive embolic members in the series are closer together. In an example, a series of embolic members can be delivered into a net or mesh, wherein embolic members in the series are progressively closer together (as one progresses along the series in a distal to proximal manner). In an example, a series of embolic members can be delivered into a net or mesh, wherein successive embolic members in the series are farther apart from each other. In an example, a series of embolic members can be delivered into a net or mesh, wherein embolic members in the series are progressively farther apart (as one progresses along the series in a distal to proximal manner).

In an example, a series of embolic members can be delivered into a net or mesh, wherein successive embolic members in the series decrease in durometer. In an example, a series of embolic members can be delivered into a net or mesh, wherein embolic members in the series have progressively lower durometer values (as one progresses along the series in a distal to proximal manner). In an example, a series of embolic members can be delivered into a net or mesh, wherein successive embolic members in the series increase in durometer. In an example, a series of embolic members can be delivered into a net or mesh, wherein embolic members in the series have progressively higher durometer values (as one progresses along the series in a distal to proximal manner).

In an example, a series of embolic members can be delivered into a net or mesh, wherein successive embolic members in the series are made of different materials. In an example, a series of embolic members can be delivered into a net or mesh, wherein successive embolic members in the series are made of different materials, wherein these materials differ in porosity. In an example, a series of embolic members can be delivered into a net or mesh, wherein successive embolic members in the series are made of different materials, wherein these materials differ in radiopacity. In an example, a series of embolic members can be delivered into a net or mesh, wherein successive embolic members in the series are made of different materials, wherein these materials differ in stiffness. In an example, a series of embolic members can be delivered into a net or mesh, wherein successive embolic members in the series are made of different materials, wherein these materials differ in durometer.

In an example, a series of embolic members can be delivered into a net or mesh, wherein successive embolic members in the series decrease in porosity. In an example, a series of embolic members can be delivered into a net or mesh, wherein embolic members in the series become progressively less porous (as one progresses along the series in a distal to proximal manner). In an example, a series of embolic members can be delivered into a net or mesh, wherein successive embolic members in the series increase in porosity. In an example, a series of embolic members can be delivered into a net or mesh, wherein embolic members in the series become progressively more porous (as one progresses along the series in a distal to proximal manner).

In an example, a series of embolic members can be delivered into a net or mesh, wherein successive embolic members in the series differ in shape. In an example, a series of embolic members can be delivered into a net or mesh, wherein successive embolic members in the series differ in their degree of convexity. In an example, a series of embolic members can be delivered into a net or mesh, wherein successive embolic members in the series differ in their degree of concavity.

In an example, a series of embolic members can be delivered into a net or mesh, wherein successive embolic members in the series decrease in size. In an example, a series of embolic members can be delivered into a net or mesh, wherein embolic members in the series become progressively smaller (as one progresses along the series in a distal to proximal manner). In an example, a series of embolic members can be delivered into a net or mesh, wherein successive embolic members in the series increase in size. In an example, a series of embolic members can be delivered into a net or mesh, wherein embolic members in the series become progressively larger (as one progresses along the series in a distal to proximal manner).

In an example, embolic members can be soft, compressible members such as microsponges or blobs of gel. In an example, embolic members can be made from sponge, foam, or gel. In an example, embolic members can be hard, uncompressible members such as hard polymer spheres or beads. In an example, embolic members can be made from one or more materials selected from the group consisting of: cellulose, collagen, acetate, alginic acid, carboxy methyl cellulose, chitin, collagen glycosaminoglycan, divinylbenzene, ethylene glycol, ethylene glycol dimethylmathacrylate, ethylene vinyl acetate, hyaluronic acid, hydrocarbon polymer, hydroxyethylmethacrylate, methlymethacrylate, polyacrylic acid, polyamides, polyesters, polyolefins, polysaccharides, polyurethane, polyvinyl alcohol, silicone, urethane, and vinyl stearate.

In an example, embolic members can have a shape selected from the group consisting of: ball or sphere, ovoid, ellipsoid, and polyhedron. In an example, embolic members can have a Shore 00 value, indicative of softness or hardness, within a range of 5 to about 50. In an example, embolic members can have a diameter or like size within a range of 50 micrometers to 2000 micrometers. In an example, differently-sized embolic members can be used. In an example two or more different sizes of embolic members can be inserted into a net or mesh to occlude an aneurysm. In an example, embolic members can include small balls and large balls. In an example, it may be advantageous to first fill a net or mesh with larger balls and then continue filling the net or mesh with smaller balls. In another example, it may be advantageous to first fill a net or mesh with smaller balls and then continue filling the net or mesh with larger balls.

In an example, an intrasaccular aneurysm occlusion device can be filled with a string of pearls string (or wire) connected sequence of embolic members. In an example, an intrasaccular aneurysm occlusion device can include a series of embolic members which are connected by a strand. In an example, a device can include a string of pearls series of embolic members which are linked by a strand (e.g. a thin flexible member). In an example, a device can include a string of pearls series of embolic members which are centrally linked by a strand (e.g. a thin flexible member). In an example, a string of pearls string-or-wire connected sequence of embolic members can comprise a plurality of embolic members which are separate from each other, but pair-wise connected to each other by at least one string or wire. In an example, a plurality of members can be unevenly-spaced along the longitudinal axis of a flexible member. In an example, uneven spacing of the embolic members can be selected based on the size and shape of an aneurysm to be occluded. In an example, the distances between embolic members can vary. In an example, the space between embolic members can differ for occlusion of narrow-neck aneurysms vs. wide-neck aneurysms. In an example, distances between embolic members can become progressively shorter in a distal to proximal direction.

In an example, a line which connects embolic members can be a wire, spring, or chain. In an example, a connecting line can be a string, thread, band, fiber, or suture. In an example, embolic members can be centrally connected to each other by a connecting line. In an example, the centroids of embolic members can be connected by a connecting line. In an example, expanding arcuate embolic members can slide (e.g. up or down) along a connecting line. In an example, embolic members can slide along a connecting line, but only in one direction. In an example, a connecting line can have a ratchet structure which allows embolic members to slide closer to each other but not slide further apart. In an example, this device can further comprise a locking mechanism which stops embolic members from sliding along a connecting line. In an example, application of electromagnetic energy to a connecting line can fuse the line with the embolic members and stop them from sliding, effectively locking them in proximity to each other.

In an example, embolic members can be conveyed through a lumen to an aneurysm in a fluid flow, wherein the fluid escapes out from a net or mesh and the embolic members are retained within the net or mesh. In an example, embolic members can be conveyed through a lumen to an aneurysm by means of a moving belt or wire loop. In an example, embolic members can be conveyed through a lumen to an aneurysm by means of an Archimedes screw.

In an example, a flexible net or mesh can self-expand to a first extent after being released from a catheter into an aneurysm sac. In an example, the flexible net or mesh can further expand, to a second extent, due to pressure from the accumulation of embolic members and/or embolic material within its interior and/or distal-facing concavity. Other example variations discussed elsewhere in this disclosure or in priority-linked disclosures can also be applied to this example where relevant.

FIG. 24 shows an example of an intrasacular aneurysm occlusion device comprising: an inner embolic structure (e.g. inner mesh, net, or stent) 2401; and an outer embolic structure (e.g. outer mesh, net, or stent) 2402, wherein the inner embolic structure is inside the outer embolic structure, and wherein the inner embolic structure and the outer embolic structure are configured to be inserted and expanded within an aneurysm sac. In this example, the inner embolic structure is bowl-shaped (and/or hemispherical) and the outer embolic structure is spherical. In this example, the axes of the inner embolic structure and the outer embolic structure are aligned. Other example variations discussed elsewhere in this disclosure or priority-linked disclosures can also be applied to this example where relevant.

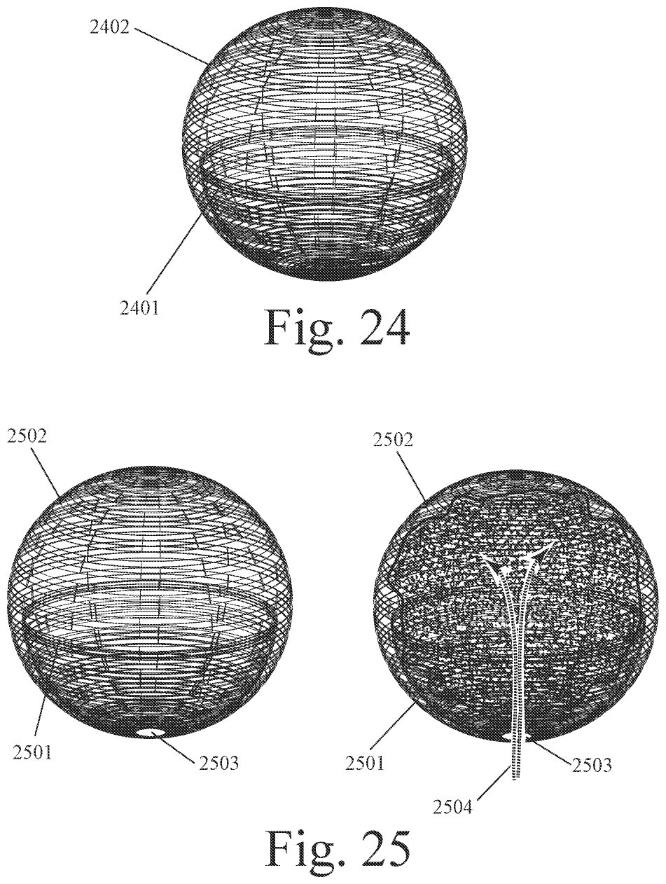

FIG. 25 shows two views, at two different times, of an example of an intrasacular aneurysm occlusion device comprising: an inner embolic structure (e.g. an inner mesh, net, or stent) 2501; an outer embolic structure (e.g. an outer mesh, net, or stent) 2502, wherein the inner embolic structure is inside the outer embolic structure, and wherein the inner embolic structure and the outer embolic structure are configured to be inserted and expanded within an aneurysm sac; an opening 2503 through the inner embolic structure; and embolic material (e.g. coils, embolic particles, string-of-pearls embolic components, or congealing material) 2504 which is inserted through the opening into the interior of the device. The left side of FIG. 25 shows this device at a first point in time, before embolic material has been inserted into the device. The right side of FIG. 25 shows this device at a second point in time, as embolic material is being inserted into the device. In this example, the inner embolic structure is bowl-shaped (and/or hemispherical) and the outer embolic structure is spherical. In this example, the axes of the inner embolic structure and the outer embolic structure are aligned. Other example variations discussed elsewhere in this disclosure or priority-linked disclosures can also be applied to this example where relevant.

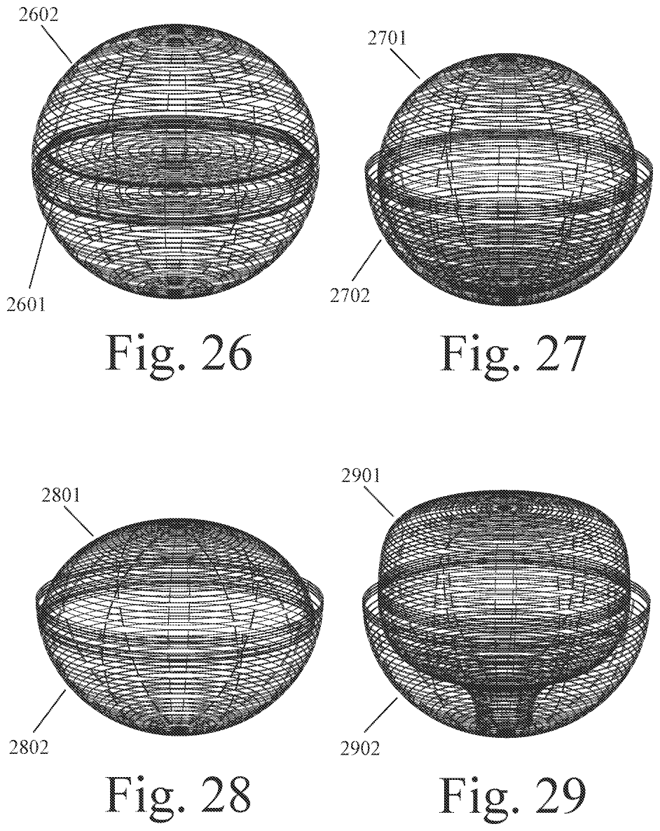

FIG. 26 shows an example of an intrasacular aneurysm occlusion device comprising: an inner embolic structure (e.g. mesh, net, braid, and/or stent) 2601 with a disk shape; and an outer embolic structure (e.g. mesh, net, braid, and/or stent) 2602 with a spherical shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 27 shows an example of an intrasacular aneurysm occlusion device comprising: an inner embolic structure (e.g. mesh, net, braid, and/or stent) 2701 with a spherical shape; and an outer embolic structure (e.g. mesh, net, braid, and/or stent) 2702 with a bowl, hemispherical, and/or paraboloidal shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 28 shows an example of an intrasacular aneurysm occlusion device comprising: an inner embolic structure (e.g. mesh, net, braid, and/or stent) 2801 with an inverted bowl, hemispherical, and/or paraboloidal shape; and an outer embolic structure (e.g. mesh, net, braid, and/or stent) 2802 with a bowl, hemispherical, and/or paraboloidal shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 29 shows an example of an intrasacular aneurysm occlusion device comprising: an inner embolic structure (e.g. mesh, net, braid, and/or stent) 2901 with an inverted jug and/or bottle shape; and an outer embolic structure (e.g. mesh, net, braid, and/or stent) 2902 with a bowl, hemispherical, and/or paraboloidal shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

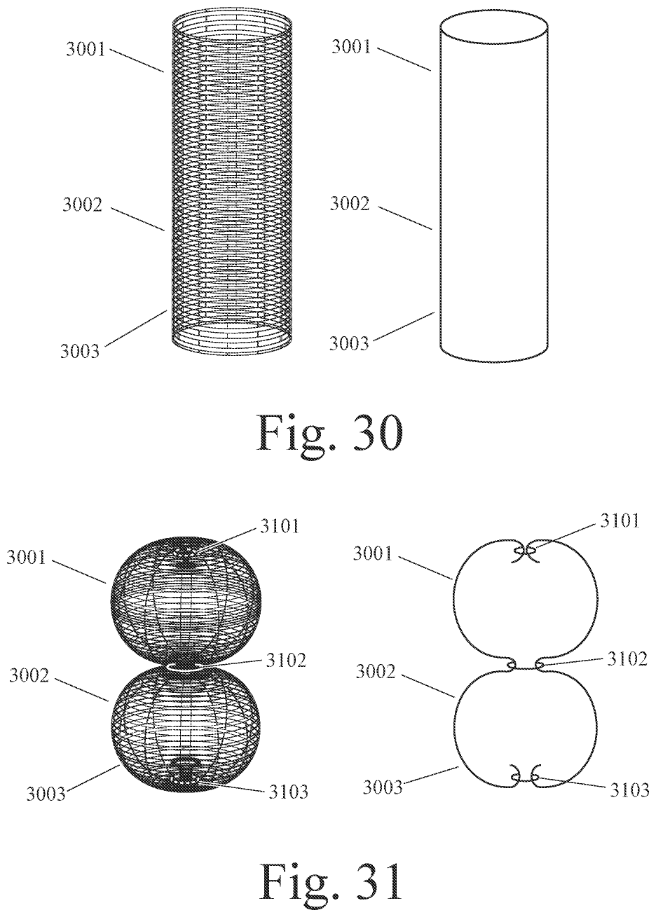

FIGS. 30 through 34 show multiple views of an example of an intrasacular aneurysm occlusion device whose final deployment configuration comprises: a proximal bowl-shaped mesh or net which is configured to be inserted into an aneurysm sac and expanded to span a neck of the aneurysm sac; and a distal globular mesh or net which is configured to be inserted into the aneurysm sac between the proximal bowl-shaped mesh or net and a distal dome of the aneurysm sac to hold the proximal bowl-shaped mesh or net against the neck of the aneurysm. FIGS. 30 through 34 show the device at five different points in time. In each figure, two views of the device are shown. A 3D wire-mesh view of the device is shown on the left side of the figure. A longitudinal cross-sectional (perimeter) view of the device is shown on the right side of the figure.

The intrasacular aneurysm occlusion device (shown in FIGS. 30 through 34) is an example of the type of device introduced in the “(G)” branch of the device taxonomy shown in FIG. 1. In this type of device, the globular portion and the bowl-shaped portion are part of the same continuous mesh or net, the bowl-shaped portion is created by inverting or everting the mesh or net, the bowl-shaped portion has two layers, and the globular portion has a distal inversion. The five views provided in FIGS. 30 through 34 show how this device is formed from a tubular mesh, radially-compressed and elongated for delivery through a catheter, inserted in and radially-expanded within an aneurysm sac, and then (partially) inverted or everted within the aneurysm sac.

FIG. 30 shows this device at a first point in time, starting out as a tubular mesh or net. Three different portions of this tubular mesh or net are labeled to better track how they are moved in subsequent figures. These portions of the tubular mesh or net are: a distal portion 3001 of the tubular mesh or net; a middle (middle-to-proximal in this example) portion 3002 of the tubular mesh or net; and a proximal portion 3003 of the tubular mesh or net. The left side of this figure shows a 3D wire-mesh view of the tube. The right side of the figure shows a longitudinal cross-sectional (perimeter) view of the tube.

In an example, a tubular mesh can have longitudinal variation in one or more mesh characteristics selected from the group consisting of: durometer, elasticity, flexibility, compliance, density, porosity, thickness, resilience, and width. In an example, a proximal portion of the tubular mesh can have a lower durometer, be less elastic, be less flexible, be less compliance, be more dense, be less porous, be thicker, be more resilient, and/or be wider than a distal portion of the tubular mesh. In an example, a proximal half of the tubular mesh can have a lower durometer, be less elastic, be less flexible, be less compliance, be more dense, be less porous, be thicker, be more resilient, and/or be wider than a distal half of the tubular mesh. In an example, a proximal third of the tubular mesh can have a lower durometer, be less elastic, be less flexible, be less compliance, be more dense, be less porous, be thicker, be more resilient, and/or be wider than a distal third of the tubular mesh.

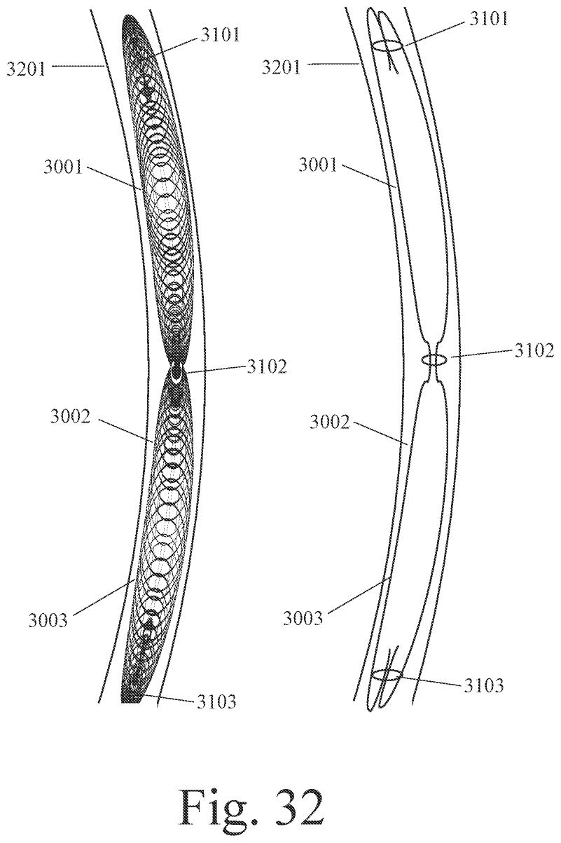

FIG. 31 shows this device at a second point in time, after: the distal end of the tubular mesh or net has been inverted and radially-constrained by a distal ring (e.g. distal ring, band, cylinder, wire, or string) 3101; the middle of the tubular mesh has been radially-constrained by a middle ring (e.g. middle ring, band, cylinder, wire, or string) 3102; and the proximal end of the tubular mesh or net has been inverted and radially-constrained by proximal ring (e.g. proximal ring, band, cylinder, wire, or string) 3103. These rings and inversions transform the tubular mesh or net into two globular meshes or nets: a distal globular mesh or net; and a proximal globular mesh or net. The distal globular mesh or net is formed from the distal portion 3001 of the original tubular mesh or net. The proximal globular mesh or net is formed from the middle portion 3002 and the proximal portion 3003 of the original tubular mesh or net. The left side of this figure shows a 3D wire-mesh view of these globular meshes or nets. The right side of this figure shows a longitudinal cross-sectional (perimeter) view of these globular meshes or nets.

In this example, a distal globular mesh or net has a generally spherical, ball, ellipsoidal, or oblate spheroid shape. In this example, a distal globular mesh or net has a distal inversion. In an example, a distal inversion can be made by inverting a distal end of a tubular mesh and then radially-constraining the inverted portion with a radial band or ring. In an example, a distal globular mesh or net can have a 3D cardioid (e.g. cardioid of rotation) shape. In an example, a distal globular mesh can have a cardioid shape if the distal inversion is larger than the one shown here. In an example, a distal inversion can extend inward between 10% and 30% of the interior diameter of a distal globular mesh or net. In an example, a distal inversion can extend inward between 20% and 55% of the interior diameter of a distal globular mesh or net. In an example, a distal inversion can extend inward between 50% and 80% of the interior diameter of a distal globular mesh or net.

FIG. 32 shows this device at a third point in time, after the device has been elongated (e.g. longitudinally-stretched) and radially-compressed for delivery through a catheter 3201 to an aneurysm sac. The device components identified in previous figures are all still present: the distal portion 3001 of the original tubular mesh or net; the middle portion 3002 of the original tubular mesh or net; the proximal portion 3003 of the original tubular mesh or net; the distal ring 3101; the middle ring 3102; and the proximal ring 3103. The left side of this figure shows a 3D wire-mesh view. The right side of this figure shows a longitudinal cross-sectional (perimeter) view.

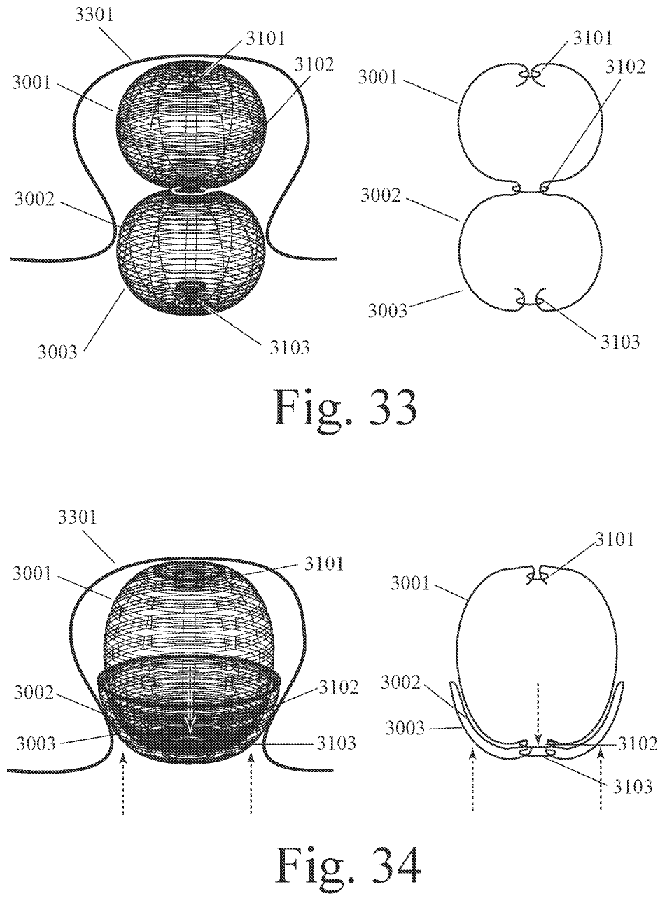

FIG. 33 shows this device at a fourth point in time, after the device has inserted into and expanded within an aneurysm sac 3301. At this time, the device has expanded back into two globular meshes or nets. Device components still include: the distal portion 3001 of the original tubular mesh or net; the middle portion 3002 of the original tubular mesh or net; the proximal portion 3003 of the original tubular mesh or net; the distal ring 3101; the middle ring 3102; and the proximal ring 3103. The left side of the figure shows a 3D wire-mesh view. The right side of the figure shows a longitudinal cross-sectional (perimeter) view.

FIG. 34 shows this device at a fifth point in time, after: the proximal globular mesh or net has been compressed into a proximal bowl-shaped mesh or net; and the distal globular mesh or net is now nested within the concavity of the proximal bowl-shaped mesh or net. In an example, this can be the final deployment configuration of the device. The device components identified in previous figures are all still present: the distal portion 3001 of the original tubular mesh or net, the middle portion 3002 of the original tubular mesh or net, the proximal portion 3003 of the original tubular mesh or net, the distal ring 3101, the middle ring 3102; and the proximal ring 3103. The left side of the figure shows a 3D wire-mesh view. The right side of the figure shows a longitudinal cross-sectional (perimeter) view.

In an example, a proximal globular mesh can be transformed into a proximal bowl-shaped mesh or net by movement of a guidewire, filament, or catheter. In an example, an operator can pull a portion of the distal globular mesh in a proximal direction or push a portion of the proximal globular mesh in a distal direction by pulling or pushing a guidewire, filament, or catheter. In an example, an operator can pull the distal globular mesh in a proximal direction or push the proximal globular mesh in a distal direction by pulling or pushing a guidewire, filament, or catheter that is attached to the distal globular mesh. In an example, an operator can pull the distal globular mesh in a proximal direction or push the proximal globular mesh in a distal direction by pulling or pushing a guidewire, filament, or catheter that is attached to the proximal globular mesh.

In an example, a guidewire, filament, string, suture, or catheter can be attached to the proximal pole of a distal globular mesh or net, wherein pulling on this guidewire, filament, string, suture, or catheter causes the distal globular mesh or net to move in a proximal direction and compress and/or fold the proximal globular mesh or net into a proximal bowl-shaped mesh or net. In an example, a guidewire, filament, string, suture, or catheter can be attached to the distal pole of a proximal globular mesh or net, wherein pulling on this guidewire, filament, string, suture, or catheter causes the proximal globular mesh to compress and/or fold into a proximal bowl-shaped mesh or net. In an example, a catheter or other longitudinal member can be attached to the proximal surface of a proximal globular mesh or net, wherein pushing on this catheter or other longitudinal member causes the proximal globular mesh to compress and/or fold into a proximal bowl-shaped mesh or net. In an example, application of electrical energy to the device causes the proximal globular mesh to compress and/or fold into a proximal bowl-shaped mesh or net.

In this example, a proximal bowl-shaped mesh or net has two layers in the final device configuration shown in FIG. 34. In this example, a proximal bowl-shaped mesh or net has a distal-facing concavity. In this example, a proximal bowl-shaped mesh or net has a hemispherical or parabolic shape. In this example, a proximal bowl-shaped mesh or net is radially symmetric. In this example, a proximal bowl-shaped mesh or net with two layers is made by folding, inverting, or everting a mesh or net over itself. In an example, a circumferential (e.g. annular) portion of the proximal globular mesh shown in FIG. 33 can be more flexible, lower durometer, thinner, and/or more elastic than other portions of original tubular mesh or net so that the proximal globular mesh or net more easily folds and/or compresses into a proximal bowl-shaped mesh or net when a guidewire, filament, or catheter is moved.

In this example, a distal globular mesh or net is nested within a distal-facing concavity of a proximal bowl-shaped mesh or net. In an example, a proximal bowl-shaped mesh or net can be around the perimeter of part of a distal globular mesh or net. In an example, a proximal bowl-shaped mesh or net can be around a proximal portion of the perimeter of part of a distal globular mesh or net. In an example, a proximal bowl-shaped mesh or net can be around the proximal half of the perimeter of part of a distal globular mesh or net. In this example, a proximal bowl-shaped mesh or net and a distal globular mesh or net are coaxial. In an example, the proximal-to-distal longitudinal axis of a proximal bowl-shaped mesh or net and a portion of the proximal-to-distal longitudinal axis of a distal globular mesh or net can overlap.

In an example, greatest diameter of a proximal bowl-shaped mesh or net can be a first distance and the greatest diameter of a distal globular mesh can be a second distance, wherein the first distance is between 5% and 20% larger than the second distance. In an example, greatest diameter of a proximal bowl-shaped mesh or net can be a first distance and the greatest diameter of a distal globular mesh can be a second distance, wherein the first distance is between 10% and 40% larger than the second distance. In an example, there can be a resilient ring or band around the greatest circumference of the proximal bowl-shaped mesh which is more resilient, less flexible, thicker, and/or less compliant than the rest of the proximal bowl-shaped mesh. In an example, there can be a resilient ring or band around the greatest circumference of the proximal bowl-shaped mesh which is more resilient, less flexible, thicker, and/or less compliant than the greatest circumference of the distal globular mesh.

Although not shown here in FIGS. 30 through 34, in an example embolic members (e.g. coils, hydrogels, beads, microsponges, string-of-pearl embolics) or congealing embolic material (e.g. embolic liquid or gel) can be inserted into a distal globular mesh or net. In an example, embolic members (e.g. coils, hydrogels, beads, microsponges, string-of-pearl embolics) or congealing embolic material (e.g. embolic liquid or gel) can be inserted into a distal globular mesh or net through holes (e.g. holes, lumens, or openings) in the proximal bowl-shaped mesh or net and in the distal globular mesh or net. In an example, insertion of the embolic members and/or congealing embolic material can cause the distal globular mesh or net to (further) expand to better fit the contours of an (irregularly-shaped) aneurysm sac. In an example, embolic material can be pushed into a distal globular mesh or net using a pusher wire, a flow of liquid, a magnetic field, a rotating helical member (e.g. an Archimedes Screw), or a conveyor belt mechanism.

Although not shown here in FIGS. 30 through 34, in an example a device can further comprise a closure mechanism which can close a lumen (e.g. hole, lumen, or opening) in a proximal bowl-shaped mesh or net and/or a distal globular mesh or net after embolic members and/or congealing material has been inserted through it. In an example, a closure mechanism can be remotely activated by the operator of the device. In an example, a closure mechanism can be a multi-leaflet valve, a one-way valve, a plug, an elastic band, a spring-loaded lid, a solenoid, or two or more openings which can be selectively aligned (opened) or misaligned (closed). Example variations discussed elsewhere in this disclosure or priority-linked disclosures can also be applied to this example where relevant.

In this example, a proximal bowl-shaped mesh or net and a distal globular mesh or net are formed from the same continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a single continuous structure. In an example, a proximal bowl-shaped mesh or net and a distal globular mesh or net can be made by radially-constraining (e.g. pinching), inverting (e.g. folding and overlapping inward), and/or everting (e.g. folding and overlapping outward) a tubular mesh or net. In this example, a proximal bowl-shaped mesh or net and a distal globular mesh or net can be made from a sequence of two globular meshes or nets, wherein the proximal one is compressed to form the proximal bowl-shaped mesh and the distal one is compressed into the concavity of the proximal bowl-shaped mesh.

With respect to methods, FIGS. 30 through 34 show an example of how an intrasacular aneurysm occlusion device can be formed and deployed as follows: (a) forming two globular meshes from a tubular mesh by inverting the distal end of the tubular mesh, radially-constraining a middle portion of the tubular mesh or net, and inverting the proximal end of the tubular mesh; (b) longitudinally-stretching and radially-compressing the two globular meshes for delivery through a catheter to an aneurysm sac; (c) inserting and radially-expanding the two globular meshes within the aneurysm sac; then (d) forming a proximal bowl-shaped mesh and a distal globular mesh within the aneurysm sac by compressing and/or folding the proximal (e.g. closest to aneurysm neck) globular mesh into bowl-shaped mesh, wherein the distal globular mesh is nested within the concavity of the proximal bowl-shaped mesh.

With respect to methods, an intrasacular aneurysm occlusion device can be formed and deployed as follows: (a) forming two globular meshes from a tubular mesh by radially-constraining the distal end of the tubular mesh, radially-constraining a middle portion of the tubular mesh or net, and radially-constraining the proximal end of the tubular mesh; (b) longitudinally-stretching and radially-compressing the two globular meshes for delivery through a catheter to an aneurysm sac; (c) inserting and radially-expanding the two globular meshes within the aneurysm sac; then (d) forming a proximal bowl-shaped mesh and a distal globular mesh within the aneurysm sac by compressing and/or folding the proximal (e.g. closest to aneurysm neck) globular mesh into bowl-shaped mesh, wherein the distal globular mesh is nested within the concavity of the proximal bowl-shaped mesh. Example variations discussed elsewhere in this disclosure or priority-linked disclosures can also be applied to this example where relevant.

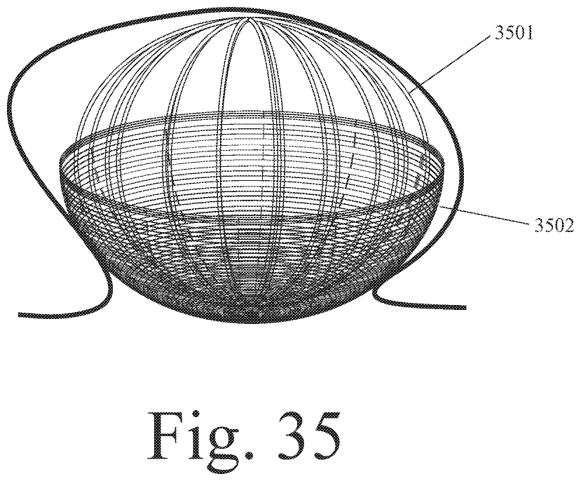

FIG. 35 shows an example of an aneurysm occlusion device comprising an intrasacular arcuate distal stent 3501 and an intrasacular arcuate proximal stent 3502, wherein the proximal stent has a concavity into which a portion of the distal stent fits when the device is deployed within an aneurysm sac. In an example, an aneurysm occlusion device can comprise a distal ball stent and a proximal hemispherical stent, wherein both are expanded and overlap each other when they are deployed within an aneurysm sac. In an example, a distal stent can be an ellipsoid when it is expanded and a proximal stent can be a section of an ellipsoid when it is expanded. In an example, distal and proximal stents which comprise this device can be nested. Example variations discussed elsewhere in this disclosure or priority-linked disclosures can also be applied to this example where relevant.

In an example, an intrasacular aneurysm occlusion device can comprise: a proximal bowl-shaped mesh or net which is configured to span a neck of an aneurysm sac; and a distal globular mesh or net which is configured to be between the proximal bowl-shaped mesh or net and a distal dome of the aneurysm sac to hold the proximal bowl-shaped mesh or net against the neck of the aneurysm sac. In an example, the proximal bowl-shaped mesh or net can have two layers and/or double thickness. In an example, the proximal bowl-shaped mesh or net can be formed by compressing, folding, and/or inverting a spherical or ellipsoidal mesh or net. In an example, the proximal bowl-shaped mesh or net can be formed by folding and/or inverting a spherical or ellipsoidal mesh or net along its central circumference.

In an example, the distal globular mesh or net can have a spherical, oblate spheroidal, or ellipsoidal shape. In an example, the distal globular mesh or net can have a cardioid shape. In an example, the distal globular mesh or net can have a distal inversion. In an example, the proximal bowl-shaped mesh or net and the distal globular mesh or net can have a first configuration in which they do not overlap as they travel through a catheter toward an aneurysm sac and a second configuration in which they overlap after they have been deployed in the aneurysm sac. In an example, the proximal bowl-shaped mesh or net and the distal globular mesh or net can have a first configuration in which they are not nested as they travel through a catheter toward an aneurysm sac and a second configuration in which they are nested after they have been deployed in the aneurysm sac.

In an example, the proximal bowl-shaped mesh or net and the distal globular mesh or net can be moved toward each other when a user pulls, rotates, or pushes a wire or string. In an example, there can be a central opening, hole, tube, and/or lumen on the central longitudinal axis of the distal globular mesh or net through which embolic material and/or members are inserted into the distal globular mesh or net.

In an example, an aneurysm occlusion device can comprise: an intrasacular arcuate proximal stent; and an intrasacular arcuate distal stent, wherein the proximal stent has a concavity into which a portion of the distal stent fits when the device is deployed within an aneurysm sac. In an example, the distal stent can be spherical when expanded and the proximal stent can be hemispherical when expanded. In an example, the distal stent can be ellipsoidal when expanded and the proximal stent can be a section of an ellipsoid when expanded. In an example, the device can have double thickness where it covers the aneurysm neck.

In an example, the distal and proximal stents do not overlap as they travel through a catheter, but they do overlap after they are deployed in an aneurysm sac. In an example, the distal and proximal stents can have a first configuration in which they are not nested as they travel through a catheter toward an aneurysm sac and a second configuration in which they are nested after they have been deployed in the aneurysm sac. In an example, the distal stent and/or the proximal stent can be connected to a wire or string. In an example, the distal and proximal stents can be moved toward each other when a user pulls, rotates, or pushes the wire or string.

In an example, a method for forming and deploying an intrasacular aneurysm occlusion device can comprise: (a) forming two globular meshes from a tubular mesh by radially-constraining a distal end of the tubular mesh, radially-constraining a middle portion of the tubular mesh, and radially-constraining a proximal end of the tubular mesh; (b) longitudinally-stretching and radially-compressing the two globular meshes for delivery through a catheter to an aneurysm sac; (c) inserting and radially-expanding the two globular meshes within the aneurysm sac; and (d) forming a proximal bowl-shaped mesh and a distal globular mesh within the aneurysm sac by compressing and/or folding the proximal globular mesh into bowl-shaped mesh, wherein the distal globular mesh is nested within the concavity of the proximal bowl-shaped mesh.

I claim:

1. An intrasacular aneurysm occlusion device comprising:
a proximal bowl-shaped mesh or net which is configured to span a neck of an aneurysm sac; and
a distal globular mesh or net which is configured to be between the proximal bowl-shaped mesh or net and a distal dome of the aneurysm sac to hold the proximal bowl-shaped mesh or net against the neck of the aneurysm sac.

2. The device in claim 1 wherein the proximal bowl-shaped mesh or net has two layers and/or double thickness.

3. The device in claim 1 wherein the proximal bowl-shaped mesh or net is formed by compressing, folding, and/or inverting a spherical or ellipsoidal mesh or net.

4. The device in claim 3 wherein the proximal bowl-shaped mesh or net is formed by folding and/or inverting a spherical or ellipsoidal mesh or net along its central circumference.

5. The device in claim 1 wherein the distal globular mesh or net has a spherical, oblate spheroidal, or ellipsoidal shape.

6. The device in claim 1 wherein the distal globular mesh or net has a cardioid shape.

7. The device in claim 1 wherein the distal globular mesh or net has a distal inversion.

8. The device in claim 1 wherein the proximal bowl-shaped mesh or net and the distal globular mesh or net have a first configuration in which they do not overlap as they travel through a catheter toward an aneurysm sac and a second configuration in which they overlap after they have been deployed in the aneurysm sac.

9. The device in claim 1 wherein the proximal bowl-shaped mesh or net and the distal globular mesh or net have a first configuration in which they are not nested as they travel through a catheter toward an aneurysm sac and a second configuration in which they are nested after they have been deployed in the aneurysm sac.

10. The device in claim 1 wherein the proximal bowl-shaped mesh or net and the distal globular mesh or net are moved toward each other when a user pulls, rotates, or pushes a wire or string.

11. The device in claim 1 wherein there is a central opening, hole, tube, and/or lumen on the central longitudinal axis of the distal globular mesh or net through which embolic material and/or members are inserted into the distal globular mesh or net.

12. An aneurysm occlusion device comprising;
an intrasacular arcuate proximal stent; and
an intrasacular arcuate distal stent, wherein the proximal stent has a concavity into which a portion of the distal stent fits when the device is deployed within an aneurysm sac.

13. The device in claim 12 wherein the distal stent is spherical when expanded and the proximal stent is hemispherical when expanded.

14. The device in claim 12 wherein the distal stent is ellipsoidal when expanded and the proximal stent is a section of an ellipsoid when expanded.

15. The device in claim 12 wherein the device has double thickness where it covers the aneurysm neck.

16. The device in claim 12 wherein the distal and proximal stents do not overlap as they travel through a catheter, but they do overlap after they are deployed in an aneurysm sac.

17. The device in claim 12 wherein the distal and proximal stents have a first configuration in which they are not nested as they travel through a catheter toward an aneurysm sac and a second configuration in which they are nested after they have been deployed in the aneurysm sac.

18. The device in claim 12 wherein the distal stent and/or the proximal stent are connected to a wire or string.

19. The device in claim 18 wherein the distal and proximal stents are moved toward each other when a user pulls, rotates, or pushes the wire or string.

20. A method for forming and deploying an intrasacular aneurysm occlusion device comprising:
forming two globular meshes from a tubular mesh by radially-constraining a distal end of the tubular mesh, radially-constraining a middle portion of the tubular mesh, and radially-constraining a proximal end of the tubular mesh;
longitudinally-stretching and radially-compressing the two globular meshes for delivery through a catheter to an aneurysm sac;
inserting and radially-expanding the two globular meshes within the aneurysm sac; and
forming a proximal bowl-shaped mesh and a distal globular mesh within the aneurysm sac by compressing and/or folding the proximal globular mesh into bowl-shaped mesh, wherein the distal globular mesh is nested within the concavity of the proximal bowl-shaped mesh.
